SEQUENCE LISTING (72 sequences)

SEQ ID NO: 1 - protein, 9 aa, synthetic construct
  REGION 1..9: note = Synthetic
GYSITSGYY

SEQ ID NO: 2 - protein, 7 aa, synthetic construct
  REGION 1..7: note = Synthetic
ITFDGAN

SEQ ID NO: 3 - protein, 12 aa, synthetic construct
  REGION 1..12: note = Synthetic
TRSSYDYDVLDY

SEQ ID NO: 4 - protein, 6 aa, synthetic construct
  REGION 1..6: note = Synthetic
QDISNF

SEQ ID NO: 5 - skipped

SEQ ID NO: 6 - protein, 9 aa, synthetic construct
  REGION 1..9: note = Synthetic
QQGHTLPYT

SEQ ID NO: 7 - protein, 6 aa, synthetic construct
  REGION 1..6: note = Synthetic
SGYYWN

SEQ ID NO: 8 - protein, 16 aa, synthetic construct
  REGION 1..16: note = Synthetic
YITFDGANNYNPSLKN

SEQ ID NO: 9 - protein, 10 aa, synthetic construct
  REGION 1..10: note = Synthetic
SSYDYDVLDY

SEQ ID NO: 10 - protein, 11 aa, synthetic construct
  REGION 1..11: note = Synthetic
RASQDISNFLN

SEQ ID NO: 11 - protein, 7 aa, synthetic construct
  REGION 1..7: note = Synthetic
YTSRLHS

SEQ ID NO: 12 - protein, 8 aa, synthetic construct
  REGION 1..8: note = Synthetic
GYSITSGY

SEQ ID NO: 13 - skipped

SEQ ID NO: 14 - protein, 8 aa, synthetic construct
  REGION 1..8: note = Synthetic
SYDYDVLD

SEQ ID NO: 15 - protein, 7 aa, synthetic construct
  REGION 1..7: note = Synthetic
SQDISNF

SEQ ID NO: 16 - protein, 6 aa, synthetic construct
  REGION 1..6: note = Synthetic
GHTLPY

SEQ ID NO: 17 - protein, 119 aa, synthetic construct
  REGION 1..119: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSS

SEQ ID NO: 18 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIK

SEQ ID NO: 19 - protein, 227 aa, synthetic construct
  REGION 1..227: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 20 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 30 bp, synthetic construct
  misc_feature 1..30: note = Synthetic
  source 1..30: mol_type = other DNA
ctccaacatcaaggaagatggcatttctag

SEQ ID NO: 22 - DNA, 30 bp, synthetic construct
  misc_feature 1..30: note = Synthetic
  source 1..30: mol_type = other DNA
ctagaaatgccatcttccttgatgttggag

SEQ ID NO: 23 - DNA, 13993 bp, Homo sapiens
  source 1..13993: mol_type = genomic DNA
tcctggcatcagttactgtgttgactcactcagtgttgggatcactcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcaggtcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctagaatggatttttcccggagccggaagccaggaggaaactacaccacactaaaacattgtctacagctccagatgtttctcattttaaacaactttccactgacaacgaaagtaaagtaaagtattggatttttttaaagggaacatgtgaatgaatacacaggacttattatatcagagtgagtaatcggttggttggttgattgattgattgattgatacattcagcttcctgctgctagcaatgccacgatttagatttaatgatgcttcagtggaaatcaatcagaaggtattctgaccttgtgaacatcagaaggtattttttaactcccaagcagtagcaggacgatgatagggctggagggctatggattcccagcccatccctgtgaaggagtaggccactctttaagtgaaggattggatgattgttcataatacataaagttctctgtaattacaactaaattattatgccctcttctcacagtcaaaaggaactgggtggtttggtttttgttgcttttttagatttattgtcccatgtgggatgagtttttaaatgccacaagacataatttaaaataaataaactttgggaaaaggtgtaaaacagtagccccatcacatttgtgatactgacaggtatcaacccagaagcccatgaactgtgtttccatcctttgcatttctctgcgagtagttccacacaggtttgtaagtaagtaagaaagaaggcaaattgattcaaatgttacaaaaaaacccttcttggtggattagacaggttaaatatataaacaaacaaacaaaaattgctcaaaaaagaggagaaaagctcaagaggaaaagctaaggactggtaggaaaaagctttactctttcatgccattttatttctttttgatttttaaatcattcattcaatagataccaccgtgtgacctataattttgcaaatctgttacctctgacatcaagtgtaattagcttttggagagtgggctgacatcaagtgtaattagcttttggagagtgggttttgtccattattaataattaattaattaacatcaaacacggcttctcatgctatttctacctcactttggttttggggtgttcctgataattgtgcacacctgagttcacagcttcaccacttgtccattgcgttattttctttttcctttataattctttctttttccttcataattttcaaaagaaaacccaaagctctaaggtaacaaattaccaaattacatgaagatttggtttttgtcttgcatttttttcctttatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccaagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaaatatatgccttactattgtattatagtactgctttactgtgtatctcaataaagcacgcagttatgttac

SEQ ID NO: 24 - DNA, 233 bp, Homo sapiens
  source 1..233: mol_type = genomic DNA
ctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaag

SEQ ID NO: 25 - DNA, 182 bp, Homo sapiens
  source 1..182: mol_type = genomic DNA
atgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacag

SEQ ID NO: 26 - DNA, 213 bp, Homo sapiens
  source 1..213: mol_type = genomic DNA
gctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcag

SEQ ID NO: 27 - DNA, 173 bp, Homo sapiens
  source 1..173: mol_type = genomic DNA
aatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagg

SEQ ID NO: 28 - DNA, 148 bp, Homo sapiens
  source 1..148: mol_type = genomic DNA
gcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaag

SEQ ID NO: 29 - DNA, 148 bp, Homo sapiens
  source 1..148: mol_type = genomic DNA
gctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaag

SEQ ID NO: 30 - DNA, 109 bp, Homo sapiens
  source 1..109: mol_type = genomic DNA
aggaagttagaagatctgagctctgagtggaaggcggtaaaccgtttacttcaagagctgagggcaaagcagcctgacctagctcctggactgaccactattggagcct

SEQ ID NO: 31 - DNA, 233 bp, Homo sapiens; identical to SEQ ID NO: 24
  source 1..233: mol_type = genomic DNA

SEQ ID NO: 32 - DNA, 118 bp, Homo sapiens
  source 1..118: mol_type = genomic DNA
gcaacaatgcaggatttggaacagaggcgtccccagttggaagaactcattaccgctgcccaaaatttgaaaaacaagaccagcaatcaagaggctagaacaatcattacggatcgaa

SEQ ID NO: 33 - DNA, 212 bp, Homo sapiens
  source 1..212: mol_type = genomic DNA
ttgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaag

SEQ ID NO: 34 - DNA, 190 bp, Homo sapiens
  source 1..190: mol_type = genomic DNA
ggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaa

SEQ ID NO: 35 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 36 - DNA, 176 bp, Homo sapiens
  source 1..176: mol_type = genomic DNA
gaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagag

SEQ ID NO: 37 - protein, 8 aa, synthetic construct
  REGION 1..8: note = Synthetic
GYSFTSYW

SEQ ID NO: 38 - protein, 8 aa, synthetic construct
  REGION 1..8: note = Synthetic
IYPGDSDT

SEQ ID NO: 39 - protein, 15 aa, synthetic construct
  REGION 1..15: note = Synthetic
ARFPYDSSGYYSFDY

SEQ ID NO: 40 - protein, 6 aa, synthetic construct
  REGION 1..6: note = Synthetic
QSISSY

SEQ ID NO: 41 - skipped

SEQ ID NO: 42 - protein, 9 aa, synthetic construct
  REGION 1..9: note = Synthetic
QQSYSTPLT

SEQ ID NO: 43 - protein, 5 aa, synthetic construct
  REGION 1..5: note = Synthetic
SYWIG

SEQ ID NO: 44 - protein, 18 aa, synthetic construct
  REGION 1..18: note = Synthetic
IIYPGDSDTRYSPSFQGQ

SEQ ID NO: 45 - protein, 13 aa, synthetic construct
  REGION 1..13: note = Synthetic
FPYDSSGYYSFDY

SEQ ID NO: 46 - protein, 11 aa, synthetic construct
  REGION 1..11: note = Synthetic
RASQSISSYLN

SEQ ID NO: 47 - protein, 7 aa, synthetic construct
  REGION 1..7: note = Synthetic
AASSLQS

SEQ ID NO: 48 - protein, 7 aa, synthetic construct
  REGION 1..7: note = Synthetic
GYSFTSY

SEQ ID NO: 49 - skipped

SEQ ID NO: 50 - protein, 11 aa, synthetic construct
  REGION 1..11: note = Synthetic
PYDSSGYYSFD

SEQ ID NO: 51 - protein, 7 aa, synthetic construct
  REGION 1..7: note = Synthetic
SQSISSY

SEQ ID NO: 52 - protein, 6 aa, synthetic construct
  REGION 1..6: note = Synthetic
SYSTPL

SEQ ID NO: 53 - protein, 9 aa, synthetic construct
  REGION 1..9: note = Synthetic
DYEKHKVYA

SEQ ID NO: 54 - protein, 122 aa, synthetic construct
  REGION 1..122: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSS

SEQ ID NO: 55 - protein, 107 aa, synthetic construct [sequence omitted]

SEQ ID NO: 56 - protein, 230 aa, synthetic construct
  REGION 1..230: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 57 - protein, 214 aa, synthetic construct
  REGION 1..214: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 7 aa, synthetic construct
  REGION 1..7: note = Synthetic
VNHKPSN

SEQ ID NO: 59 - protein, 7 aa, synthetic construct
  REGION 1..7: note = Synthetic
ASTKGPS

SEQ ID NO: 60 - protein, 6 aa, synthetic construct
  REGION 1..6: note = Synthetic
SNTKVD

SEQ ID NO: 61 - protein, 7 aa, synthetic construct
  REGION 1..7: note = Synthetic
VDKKVEP

SEQ ID NO: 62 - protein, 7 aa, synthetic construct
  REGION 1..7: note = Synthetic
VEPKSCD

SEQ ID NO: 63 - protein, 7 aa, synthetic construct
  REGION 1..7: note = Synthetic
GLVKPSQ

SEQ ID NO: 64 - protein, 7 aa, synthetic construct
  REGION 1..7: note = Synthetic
PPGKGLE

SEQ ID NO: 65 - protein, 7 aa, synthetic construct
  REGION 1..7: note = Synthetic
PSLKNRV

SEQ ID NO: 66 - protein, 7 aa, synthetic construct
  REGION 1..7: note = Synthetic
FSLKLSS

SEQ ID NO: 67 - protein, 7 aa, synthetic construct
  REGION 1..7: note = Synthetic
EQLKSGT

SEQ ID NO: 68 - protein, 7 aa, synthetic construct
  REGION 1..7: note = Synthetic
REAKVQW

SEQ ID NO: 69 - protein, 7 aa, synthetic construct
  REGION 1..7: note = Synthetic
VQWKVDN

SEQ ID NO: 70 - protein, 7 aa, synthetic construct
  REGION 1..7: note = Synthetic
PVTKSFN

SEQ ID NO: 71 - protein, 7 aa, synthetic construct
  REGION 1..7: note = Synthetic
YQQKPGQ

SEQ ID NO: 72 - protein, 7 aa, synthetic construct
  REGION 1..7: note = Synthetic
LEIKRTV

(19) United States
(12) Patent Application Publication
(10) Pub. No.: US 2026/0137797 A1
(43) Pub. Date: May 21, 2026

(54) MUSCLE TARGETING COMPLEXES FOR TREATING DYSTROPHINOPATHIES

(71) Applicant: Dyne Therapeutics, Inc., Waltham, MA (US)

(72) Inventors: Timothy Weeden, Waltham, MA (US); Benjamin Vieira, Waltham, MA (US); Cody A. Desjardins, Waltham, MA (US); Romesh R. Subramanian, Framingham, MA (US); Mohammed T. Qatanani, Waltham, MA (US); Brendan Quinn, Waltham, MA (US); John Najim, Waltham, MA (US); Peiyi Shen, Waltham, MA (US); Qifeng Qiu, Waltham, MA (US)

(73) Assignee: Dyne Therapeutics, Inc., Waltham, MA (US)

(21) Appl. No.: 18/706,057

(22) PCT Filed: Oct. 31, 2022
(86) PCT No.: PCT/US2022/078975

§ 371 Date: Apr. 30, 2024

Related U.S. Application Data
(60) Provisional application No. 63/274,306, filed on Nov. 1, 2021.

(51) Int. Cl. A61K 47/68; A61P 21/00; C07K 16/28

(52) U.S. Cl. CPC A61K 47/6849; A61K 47/6807; A61P 21/00; C07K 16/2881; C07K 2317/55

(57) ABSTRACT

Aspects of the disclosure relate to compositions comprising a plurality of complexes comprising an antibody (e.g., anti-TfR1 antibody) covalently linked to one or more oligonucleotides (e.g. a phosphorodiamidate morpholino oligomer), each oligonucleotide being covalently linked at a linkage site represented by a lysine (K) residue of the antibody. In some embodiments, the antibody comprises a heavy chain comprising a heavy chain variable region (VH) and a heavy chain constant region, and a light chain comprising a light chain variable region (VL) and a light chain constant region, wherein at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody.

### RELATED APPLICATION

This application claims the benefit under 35 U.S.C § 119(e) of the filing date of U.S. Provisional Application No. 63/274,306, entitled “MUSCLE TARGETING COMPLEXES FOR TREATING DYSTROPHINOPATHIES”, filed Nov. 1, 2021; the contents of which are incorporated herein by reference in their entirety.

### FIELD OF THE INVENTION

The present application relates to targeting complexes for delivering an effective amount of oligonucleotide molecular payloads to cells and uses thereof, particularly uses relating to treatment of disease.

### REFERENCE TO AN ELECTRONIC SEQUENCE LISTING

The contents of the electronic sequence listing (D082470072WO00-SEQ-CBD.xml; Size: 98,568 bytes; and Date of Creation: Oct. 31, 2022) is herein incorporated by reference in its entirety.

### BACKGROUND

Dystrophinopathies are a group of distinct neuromuscular diseases that result from mutations in dystrophin gene. Dystrophinopathies include Duchenne muscular dystrophy, Becker muscular dystrophy, and X-linked dilated cardiomyopathy. Dystrophin (DMD) is a large gene, containing 79 exons and approximately 2.6 million total base pairs. Numerous mutations in DMD, including exonic frameshift, deletion, substitution, and duplicative mutations, are able to diminish the expression of functional dystrophin, leading to dystrophinopathies.

### SUMMARY OF INVENTION

According to some aspects, the present disclosure provides compositions comprising a plurality of complexes. In some embodiments, each complex of the compositions described herein comprises an antibody (e.g., an anti-transferrin receptor 1 (TfR1)) covalently linked to one or more oligonucleotides, each oligonucleotide being covalently linked at a linkage site represented by a lysine (K) residue of the antibody. In some embodiments, the antibody comprises a heavy chain comprising a heavy chain variable region (VH) and a heavy chain constant region, and a light chain comprising a light chain variable region (VL) and a light chain constant region. In some embodiments, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody.

According to some aspects, the present disclosure provides compositions comprising a plurality of complexes, wherein each complex comprises an antibody covalently linked to one or more oligonucleotides, each oligonucleotide being covalently linked at a linkage site represented by a lysine (K) residue of the antibody, wherein the antibody comprises a heavy chain comprising a heavy chain variable region (VH) and a heavy chain constant region, and a light chain comprising a light chain variable region (VL) and a light chain constant region, and wherein, at least 80% of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. In some embodiments, at least 15% of the heavy chain constant regions of the antibodies of the complexes in the composition are independently covalently linked to an oligonucleotide at a linkage site represented by K213 (based on Kabat numbering) of the heavy chain constant region of each antibody.

In some aspects, the present disclosure provides compositions comprising a plurality of complexes, wherein each complex comprises an antibody covalently linked to one or more oligonucleotides, each oligonucleotide being covalently linked at a linkage site represented by a lysine (K) residue of the antibody, wherein the antibody comprises a heavy chain comprising a heavy chain variable region (VH) and a heavy chain constant region, and a light chain comprising a light chain variable region (VL) and a light chain constant region, wherein at least 80% of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, at least 15% of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody.

In some embodiments, the antibody is an anti-transferrin receptor (TfR1) antibody comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16. In some embodiments, the antibody is an anti-transferrin receptor (TfR1) antibody comprising: a heavy chain complementarity determining region 1 (CDR-H1) comprising a sequence as set forth in SEQ ID NOs: 37, 43, or 48, a heavy chain complementarity determining region 2 (CDR-H2) comprising a sequence as set forth in SEQ ID NOs: 38, 44, or 49, a heavy chain complementarity determining region 3 (CDR-H3) comprising a sequence as set forth in SEQ ID NOs: 39, 45, or 50, a light chain complementarity determining region 1 (CDR-L1) comprising a sequence as set forth in SEQ ID NOs: 40, 46, 51, a light chain complementarity determining region 2 (CDR-L2) comprising a sequence as set forth in SEQ ID NOs: 41 or 47, and a light chain complementarity determining region 3 (CDR-L3) comprising a sequence as set forth in SEQ ID NOs: 42 or 52. In some embodiments, the oligonucleotide is a phosphorodiamidate morpholino oligomer (PMO).

In some aspects, the present disclosure provides compositions comprising a plurality of complexes of the formula (I): [R¹]ₙ₁—R², wherein each R¹ independently comprises a group of the formula (Ia):

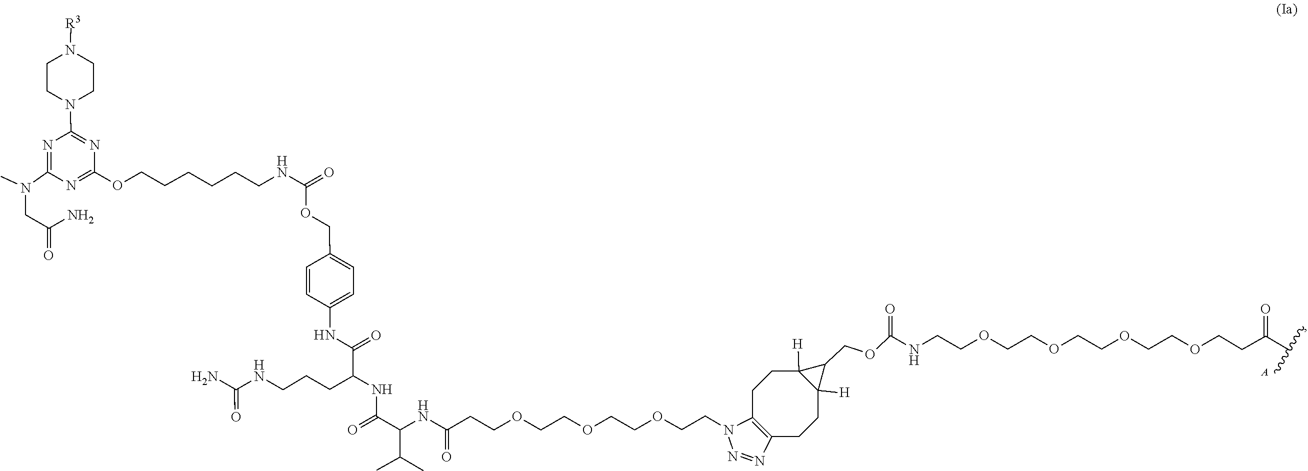

R² comprises an anti-transferrin receptor (TfR1) antibody comprising a heavy chain comprising a heavy chain variable region (VH) and a heavy chain constant region, and a light chain comprising a light chain variable region (VL) and a light chain constant region; and wherein the anti-TfR1 antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16; R³ comprises a phosphorodiamidate morpholino oligomer (PMO) comprising a nucleobase sequence of CTCCAACATCAAGGAAGATGGCATTTCTAG (SEQ ID NO: 21); wherein each R¹ is covalently linked at attachment point A to R² via a linkage site represented by a lysine (K) residue of the anti-TfR1 antibody, and wherein, at least 80% of the light chain constant regions of the anti-TfR1 antibodies of the complexes in the composition are independently covalently linked to R¹ at a linkage site represented by K188 (based on Kabat numbering) and/or a linkage site represented by K190 (based on Kabat numbering) of the light chain constant region of each antibody; and wherein in each complex, n1 is independently an integer of one or greater representing the number of instances of R¹. In some embodiments, the average value of n1 of complexes in the composition is in the range of 1 to 5.

In some aspects, the present disclosure provides compositions comprising a plurality of complexes of the formula (I): [R¹]ₙ₁—R², wherein each R¹ independently comprises a group of the formula (Ib):

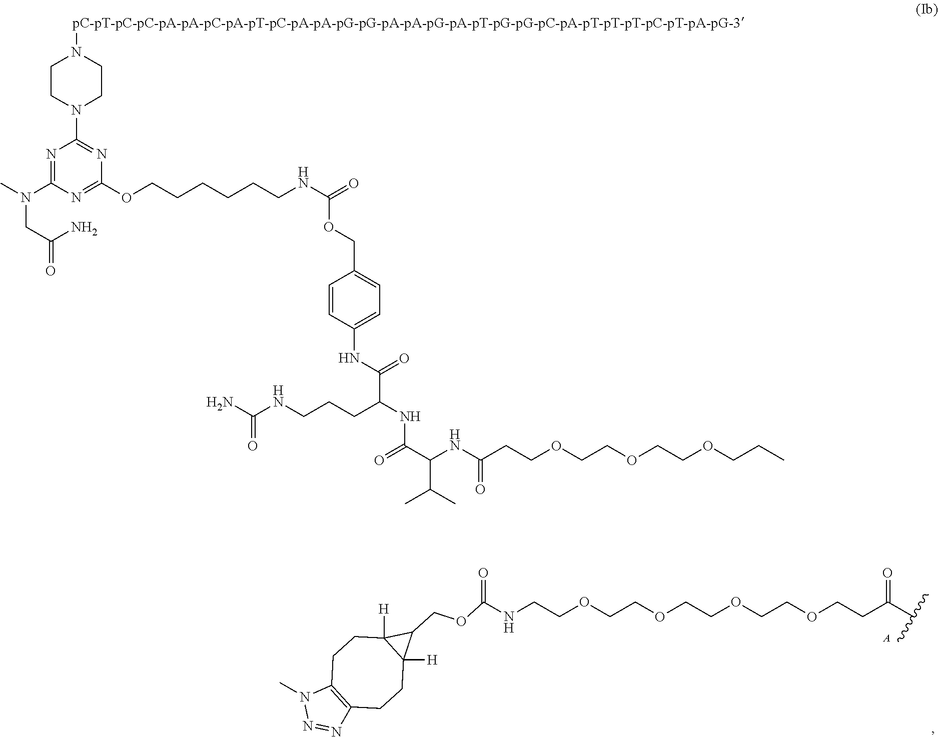

in which -p is a phosphorodiamidate linkage of a phosphorodiamidate morpholino oligomer (PMO), and wherein the PMO comprises a base sequence of CTCCAACATCAAGGAAGATGGCATTTCTAG (SEQ ID NO: 21); R² comprises an anti-transferrin receptor (TfR1) antibody comprising a heavy chain comprising a heavy chain variable region (VH) and a heavy chain constant region, and a light chain comprising a light chain variable region (VL) and a light chain constant region; and wherein the anti-TfR1 antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16; and wherein each R¹ is covalently linked at attachment point A to R² via a linkage site represented by a lysine (K) residue of the anti-TfR1 antibody, and wherein, at least 80% of the light chain constant regions of the anti-TfR1 antibodies of the complexes in the composition are independently covalently linked to R¹ at a linkage site represented by K188 (based on Kabat numbering) and/or a linkage site represented by K190 (based on Kabat numbering) of the light chain constant region of each antibody; and wherein in each complex, n1 is independently an integer of one or greater representing the number of instances of R¹. In some embodiments, the average value of n1 of the complexes of the composition is in the range of 1 to 5.

In some aspects, the present disclosure provides compositions comprising a plurality of complexes of the formula (I): [R¹]ₙ₁—R², wherein: each R¹ comprises a group of the formula (Ic):

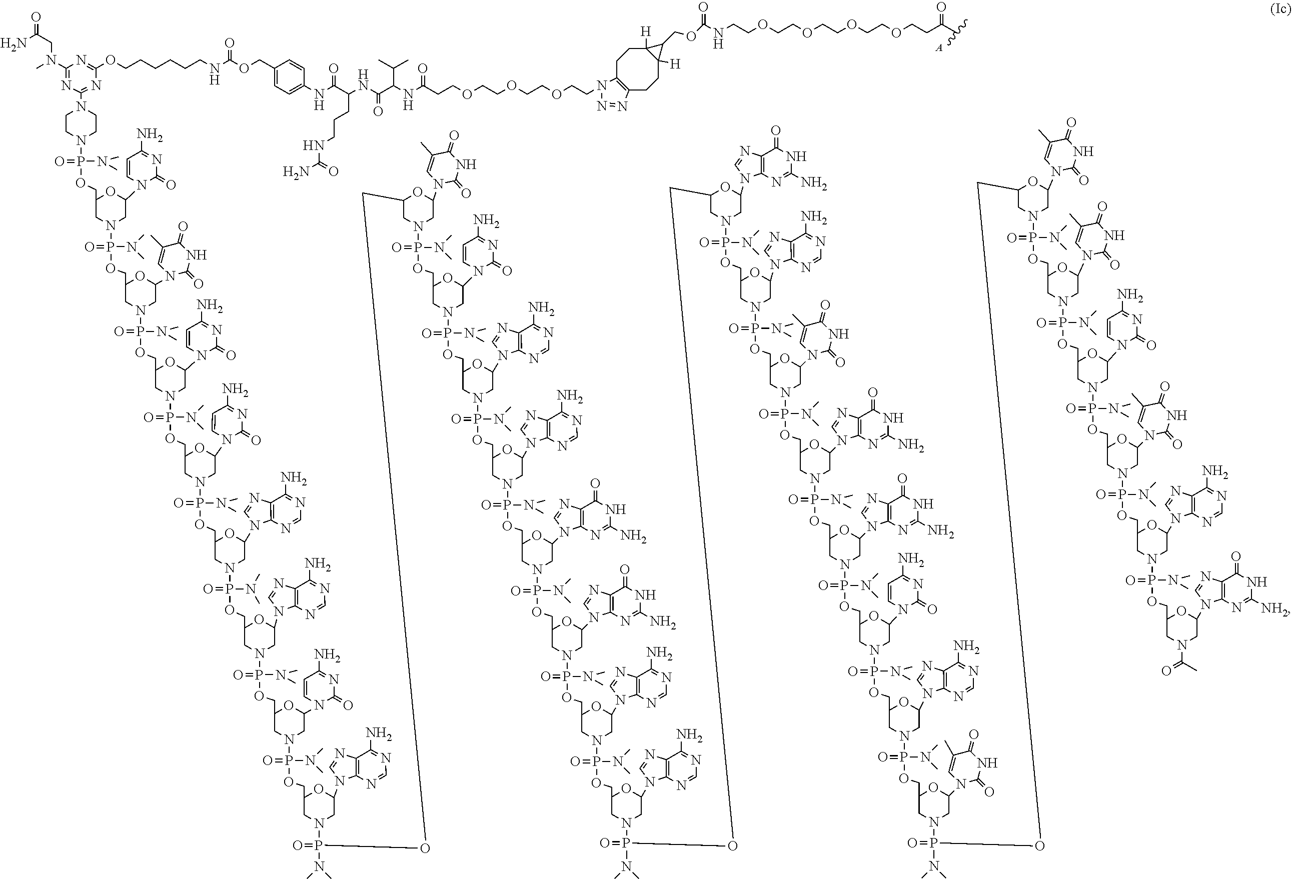

R² comprises an anti-transferrin receptor (TfR1) antibody comprising a heavy chain comprising a heavy chain variable region (VH) and a heavy chain constant region, and a light chain comprising a light chain variable region (VL) and a light chain constant region; and wherein the anti-TfR1 antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16; and wherein each R¹ is covalently linked at attachment point A to R² via a linkage site represented by a lysine (K) residue of the anti-TfR1 antibody, and wherein, at least 80% of the light chain constant regions of the anti-TfR1 antibodies of the complexes in the composition are independently covalently linked to R¹ at a linkage site represented by K188 (based on Kabat numbering) and/or a linkage site represented by K190 (based on Kabat numbering) of the light chain constant region of each antibody; and wherein in each complex, n1 is independently an integer of one or greater representing the number of instances of R¹. In some embodiments, the average value of n1 of the complexes of the composition is in the range of 1 to 5. In some embodiments, 85%-95% of the light chain constant regions of the anti-TfR1 antibodies of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, 90-95% of the light chain constant regions of the anti-TfR1 antibodies of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, at least 15% of the heavy chain constant regions of the anti-TfR1 antibodies of the complexes in the composition are independently covalently linked to R¹ at a linkage site represented by K213 (based on Kabat numbering) of the heavy chain constant region of each antibody. In some embodiments, 15-45% of the heavy chain constant regions of the anti-TfR1 antibodies of the complexes in the composition are independently covalently linked to R¹ at a linkage site represented by K213 (based on Kabat numbering) of the heavy chain constant region of each antibody.

In some embodiments, the antibody is a Fab fragment, a full-length IgG, a Fab′ fragment, or a F(ab′)2 fragment. In some embodiments, the antibody is a Fab fragment. In some embodiments, the VH comprises an amino acid sequence at least 85% identical to SEQ ID NO: 17; and/or wherein the VL comprises an amino acid sequence at least 85% identical to SEQ ID NO: 18. In some embodiments, the antibody comprises a heavy chain variable region (VH) comprising the amino acid sequence of SEQ ID NO: 17 and a light chain variable region (VL) comprising the amino acid sequence of SEQ ID NO: 18. In some embodiments, the heavy chain comprises the amino acid sequence of SEQ ID NO: 19 and a light chain comprising the amino acid sequence of SEQ ID NO: 20.

In some aspects, the present disclosure provides methods of promoting expression or activity of a dystrophin protein in a subject, comprising administering to the subject an effective amount of the composition provided herein. In some aspects, the present disclosure provides methods of treating Duchenne Muscular Dystrophy (DMD) in a subject, comprising administering to the subject an effective amount of the composition provided herein. In some embodiments, the subject has a mutated dystrophin allele comprising a mutation amenable to exon 51 skipping. In some embodiments, the mutated dystrophin allele comprises a frameshift mutation in exon 51. In some embodiments, the complexes promote expression or activity of dystrophin protein in the subject. In some embodiments, the dystrophin protein is a truncated dystrophin protein.

In some aspects, the present disclosure provides methods of determining a drug to antibody ratio (DAR) range of a first plurality of complexes, each complex comprising an antibody covalently linked to one or more oligonucleotides via a linker, wherein each linker comprises one or more protease cleavage sites, the method comprising: (i) removing the one or more oligonucleotides from the antibodies to which they are covalently linked of the first plurality of complexes by cleaving at least one of the one or more protease cleavage sites of the linker, wherein cleaving at least one of the one or more protease cleavage sites of the linker results in a second plurality of complexes, each complex comprising an antibody covalently linked to one or more partial linkers; (ii) obtaining the second plurality of complexes resulting from step (i); (iii) determining the masses of the complexes obtained in step (ii) via mass spectrometry; and (iv) determining the DAR range of the complexes obtained in step (ii); wherein a detected mass by mass spectrometry corresponding to the mass of the antibody plus the mass of n1 partial linkers indicates a DAR of n1, wherein n1 is an integer of one or greater.

In some aspects, the present disclosure provides methods of analyzing a first plurality of complexes, each complex comprising an antibody covalently linked to one or more oligonucleotides via a linker, wherein each linker comprises one or more protease cleavage sites, the method comprising: (i) removing the one or more oligonucleotides from the antibodies to which they are covalently linked of the first plurality of complexes by cleaving at least one of the one or more protease cleavage sites in the linker, wherein cleaving at least one of the one or more protease cleavage sites in the linker results in a second plurality of complexes, each complex comprising an antibody covalently linked to one or more partial linkers, and wherein the antibody remains intact; (ii) obtaining the second plurality complexes resulting from step (i); (iii) digesting the antibodies of complexes obtained in (ii) with a protease to obtain fragments of the antibodies; and (iv) determining the mass of the fragments of the antibodies obtained in step (iii) via mass spectrometry to identify the fragments covalently linked one or more partial linkers. In some embodiments, each complex of the first plurality of complexes comprises a structure of the formula (I): [R¹]ₙ₁—R², wherein: each R¹ comprises a group of the formula (Ic):

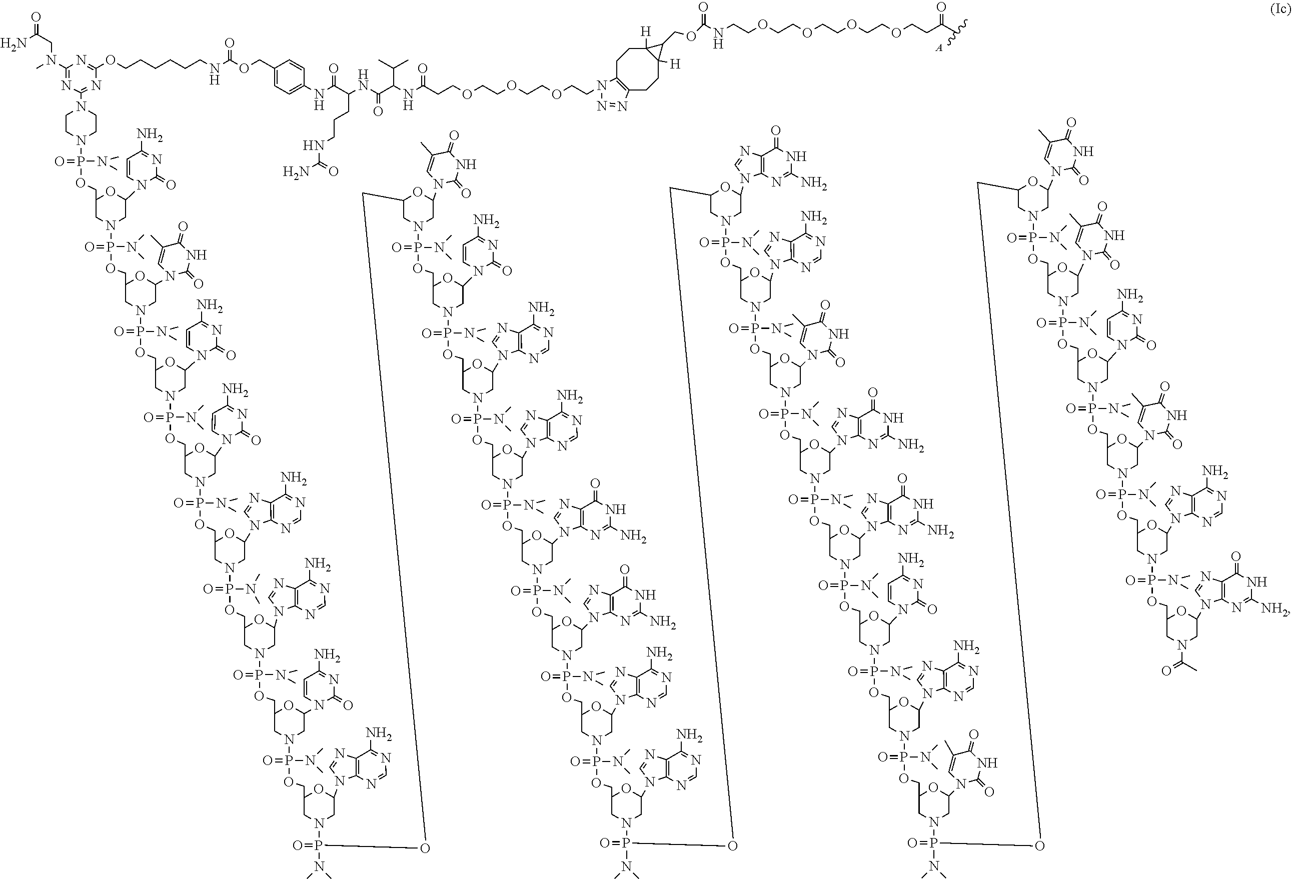

R² comprises an anti-transferrin receptor (TfR1) antibody comprising a heavy chain comprising a heavy chain variable region (VH) and a heavy chain constant region, and a light chain comprising a light chain variable region (VL) and a light chain constant region; and wherein the anti-TfR1 antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16; and wherein each R¹ is covalently linked at attachment point A to R² via a linkage site represented by a lysine (K) residue of the anti-TfR1 antibody, and wherein, at least 80% of the light chain constant regions of the anti-TfR1 antibodies of the complexes in the composition are independently covalently linked to at a linkage site represented by K188 (based on Kabat numbering) and/or a linkage site represented by K190 (based on Kabat numbering) of the light chain constant region of each antibody; and wherein in each complex, n1 is independently an integer of one or greater representing the number of instances of R¹. In some embodiments, the average value of n1 of the complexes of the composition is in the range of 1 to 5. In some embodiments, the cleaving of step (i) is carried out with papain. In some embodiments, the digesting of step (iii) is carried out with a chymotrypsin.

### BRIEF DESCRIPTION OF THE DRAWINGS

FIGS. 1A-1B show activities of anti-TfR1 Fab-oligonucleotide conjugate in inducing DMD exon 51 skipping in DMD patient myotubes. The anti-TfR1 Fab-oligonucleotide conjugate contains an anti-TfR1 Fab having the VH/VL sequences shown in Table 2 covalently linked (through lysine conjugation) via a linker comprising a valine-citrulline sequence to a DMD exon 51-skipping oligonucleotide (SEQ ID NO: 21). FIG. 1A shows that a composition comprising anti-TfR1 Fab-oligonucleotide conjugate resulted in enhanced exon skipping compared to the same DMD exon 51-skipping oligonucleotide that is not covalently linked to a Fab in DMD patient myotubes. FIG. 1B shows that anti-TfR1 Fab-oligonucleotide conjugate resulted in dose-dependent exon 51 skipping following treatment with anti-TfR1 Fab conjugate to a final concentration of 2.5 μM (low), 5 μM (medium), and 10 μM (high) oligonucleotide equivalent.

FIG. 2 shows a mass spectrum indicating the intact mass of unlinked anti-TfR1 Fabs having sequences shown in Table 2.

FIG. 3 shows a mass spectrum indicating masses of anti-TfR1 antibody-linker complexes of the compositions provided herein, after cleavage of the oligonucleotide via papain digestion. The anti-TfR1 antibody has sequences shown in Table 2.

### DETAILED DESCRIPTION OF INVENTION

According to some aspects, the present disclosure provides compositions comprising a plurality of complexes. In some embodiments, each complex of the compositions described herein comprises an antibody (e.g., an anti-transferrin receptor 1 (TfR1)) covalently linked to one or more oligonucleotides, each oligonucleotide being covalently linked at a linkage site represented by a lysine (K) residue of the antibody. In some embodiments, the antibody comprises a heavy chain comprising a heavy chain variable region (VH) and a heavy chain constant region, and a light chain comprising a light chain variable region (VL) and a light chain constant region.

In some embodiments, the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. For example, in some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody.

In some embodiments, the light chain constant regions of the antibodies of the complexes in the composition are independently covalently linked to an oligonucleotide at linkage sites represented by represented by lysine (K) residues in a sequence motif DYEKHKVYA (SEQ ID NO: 53) of the light chain constant region of each antibody. For example, in some embodiments, the linkage site is represented by the K at position 4 of a sequence motif DYEKHKVYA (SEQ ID NO: 53) of the light chain constant region of each antibody. In some embodiments, the linkage site is represented by the K at position 6 of a sequence motif DYEKHKVYA (SEQ ID NO: 53) of the light chain constant region of each antibody. In some embodiments, the linkage sites are represented by the K at position 4 and the K at position 6 of a sequence motif DYEKHKVYA (SEQ ID NO: 53) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in motif VNHKPSN (SEQ ID NO: 58) of the heavy chain constant region of each antibody.

In some aspects, the present disclosure provides methods of promoting expression or activity of a dystrophin protein (e.g., a truncated dystrophin protein) and/or methods of treating Duchenne Muscular Dystrophy (DMD) in a subject. In some embodiments, the methods comprise administering to the subject an effective amount of the composition comprising the complexes described herein.

In some aspects, the present disclosure further provides methods of determining a drug to antibody ratio (DAR) range of complexes in a composition, or methods of analyzing complexes in a composition.

Further aspects of the disclosure, including a description of defined terms, are provided below.

### Definitions

Administering: As used herein, the terms “administering” or “administration” means to provide a complex to a subject in a manner that is physiologically and/or (e.g., and) pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a Fab′ fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information System® http://www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. Examples of CDR definition systems are provided in Table 1.

TABLE 1
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

DMD: As used herein, the term “DMD” refers to a gene that encodes dystrophin protein, a key component of the dystrophin-glycoprotein complex, which bridges the inner cytoskeleton and the extracellular matrix in muscle cells, particularly muscle fibers. Deletions, duplications, and point mutations in DMD may cause dystrophinopathies, such as Duchenne muscular dystrophy, Becker muscular dystrophy, or cardiomyopathy. Alternative promoter usage and alternative splicing result in numerous distinct transcript variants and protein isoforms for this gene. In some embodiments, a dystrophin gene may be a human (Gene ID: 1756), non-human primate (e.g., Gene ID: 465559), or rodent gene (e.g., Gene ID: 13405; Gene ID: 24907). In addition, multiple human transcript variants (e.g., as annotated under GenBank RefSeq Accession Numbers: NM_000109.3, NM_004006.2 (SEQ ID NO: 24), NM_004009.3, NM_004010.3 and NM_004011.3) have been characterized that encode different protein isoforms.

DMD allele: As used herein, the term “DMD allele” refers to any one of alternative forms (e.g., wild-type or mutant forms) of a DMD gene. In some embodiments, a DMD allele may encode for dystrophin that retains its normal and typical functions. In some embodiments, a DMD allele may comprise one or more mutations that results in muscular dystrophy. Common mutations that lead to Duchenne muscular dystrophy involve frameshift, deletion, substitution, and duplicative mutations of one or more of 79 exons present in a dystrophin allele, e.g., exon 8, exon 23, exon 41, exon 44, exon 50, exon 51, exon 52, exon 53, or exon 55. Further examples of DMD mutations are disclosed, for example, in Flanigan K M, et al., Mutational spectrum of DMD mutations in dystrophinopathy patients: application of modern diagnostic techniques to a large cohort. Hum Mutat. 2009 December; 30 (12):1657-66, the contents of which are incorporated herein by reference in its entirety.

Dystrophinopathy: As used herein, the term “dystrophinopathy” refers to a muscle disease that results from one or more mutated DMD alleles. Dystrophinopathies include a spectrum of conditions (ranging from mild to severe) that includes Duchenne muscular dystrophy, Becker muscular dystrophy, and DMD-associated dilated cardiomyopathy (DCM). In some embodiments, at one end of the spectrum, dystrophinopathy is phenotypically associated with an asymptomatic increase in serum concentration of creatine phosphokinase (CK) and/or (e.g., and) muscle cramps with myoglobinuria. In some embodiments, at the other end of the spectrum, dystrophinopathy is phenotypically associated with progressive muscle diseases that are generally classified as Duchenne or Becker muscular dystrophy when skeletal muscle is primarily affected and as DMD-associated dilated cardiomyopathy (DCM) when the heart is primarily affected. Symptoms of Duchenne muscular dystrophy include muscle loss or degeneration, diminished muscle function, pseudohypertrophy of the tongue and calf muscles, higher risk of neurological abnormalities, and a shortened lifespan. Duchenne muscular dystrophy is associated with Online Mendelian Inheritance in Man (OMIM) Entry #310200. Becker muscular dystrophy is associated with OMIM Entry #300376. Dilated cardiomyopathy is associated with OMIM Entry X #302045.

Exonic splicing enhancer (ESE): As used herein, the term “exonic splicing enhancer” or “ESE” refers to a nucleic acid sequence motif within an exon of a gene, pre-mRNA, or mRNA that directs or enhances splicing of pre-mRNA into mRNA, e.g., as described in Blencowe et al., Trends Biochem Sci 25, 106-10. (2000), incorporated herein by reference. ESEs are splicing features. ESEs may direct or enhance splicing, for example, to remove one or more introns and/or one or more exons from a gene transcript. ESE motifs are typically 6-8 nucleobases in length. SR proteins (e.g., proteins encoded by the gene SRSF1, SRSF2, SRSF3, SRSF4, SRSF5, SRSF6, SRSF7, SRSF8, SRSF9, SRSF10, SRSF11, SRSF12, TRA2A or TRA2B) bind to ESEs through their RNA recognition motif region to facilitate splicing. ESE motifs can be identified through a number of methods, including those described in Cartegni et al., Nucleic Acids Research, 2003, Vol. 31, No. 13, 3568-3571, incorporated herein by reference.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti-transferrin receptor antibodies and antigen binding portions are provided. Such antibodies may be generated by obtaining murine anti-transferrin receptor monoclonal antibodies using traditional hybridoma technology followed by humanization using in vitro genetic engineering, such as those disclosed in Kasaian et al PCT publication No. WO 2005/123126 A2.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Morpholinos: As used herein, the term “morpholino”, also referred to as a “phosphorodiamidate morpholino oligomer”, refers to a molecular structure that contains nucleobases attached to a backbone of methylenemorpholine rings linked through a phosphorodiamidate group. In some embodiments, the oligonucleotide may be a morpholino-based compounds. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidate morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleosides (e.g., 2′-O-methyl sugar modifications, purine or pyrimidine modifications). In some embodiments, an oligonucleotide may comprise one or more modified internucleoside linkage. In some embodiments, an oligonucleotide may comprise one or more phosphorothioate linkages, which may be in the Rp or Sp stereochemical conformation.

Region of complementarity: As used herein, the term “region of complementarity” refers to a nucleotide sequence, e.g., of an oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a disease resulting from a mutated DMD gene sequence, e.g., a mutation in an exon of a DMD gene sequence. In some embodiments, a subject has a dystrophinopathy, e.g., Duchenne muscular dystrophy.

Transferrin receptor: As used herein, the term, “transferrin receptor” (also known as TFRC, CD71, p90, TFR, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

Ranges: All ranges provided in the present disclosure are inclusive of the end points.

### Complexes

Provided herein are compositions comprising a plurality of complexes. In some embodiments, each complex comprises an antibody covalently linked to one or more oligonucleotides, each oligonucleotide being covalently linked at a linkage site represented by a lysine (K) residue of the antibody. In some embodiments, the antibody comprises a heavy chain comprising a heavy chain variable region (VH) and a heavy chain constant region, and a light chain comprising a light chain variable region (VL) and a light chain constant region. In some embodiments, the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. For example, in some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. In some embodiments, the heavy chain constant regions of the antibodies of the complexes in the composition are independently covalently linked to an oligonucleotide at a linkage site represented by K213 (based on Kabat numbering) of the heavy chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody.

In some embodiments, the light chain constant regions of the antibodies of the complexes in the composition are independently covalently linked to an oligonucleotide at linkage sites represented by represented by lysine (K) residues in a sequence motif DYEKHKVYA (SEQ ID NO: 53) of the light chain constant region of each antibody. For example, in some embodiments, the complexes are covalently linked to an oligonucleotide at a linkage site represented by the K at position 4 in a sequence motif DYEKHKVYA (SEQ ID NO: 53) of the light chain constant region. In some embodiments, the complexes are covalently linked to an oligonucleotide at a linkage site represented by the K at position 6 in a sequence motif DYEKHKVYA (SEQ ID NO: 53) of the light chain constant region. In some embodiments, the complexes are covalently linked to an oligonucleotide at a linkage site represented the K at position 4 and at a linkage site represented by the K at position 6 in a sequence motif DYEKHKVYA (SEQ ID NO: 53) of the light chain constant region. In some embodiments, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, the heavy chain constant regions of the antibodies of the complexes in the composition are independently covalently linked to an oligonucleotide at a linkage site represented by the lysine (K) residue in a sequence a sequence motif VNHKPSN (SEQ ID NO: 58) of the heavy chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in motif VNHKPSN (SEQ ID NO: 58) of the heavy chain constant region of each antibody.

Complexes of the compositions described herein generally comprise a linker that covalently links an antibody (e.g., an anti-TfR1 antibody) described herein to an oligonucleotide (e.g., a PMO) at a linkage site represented by a lysine (K) residue of the antibody. In some embodiments, in each complex, each oligonucleotide is covalently linked at a linkage site represented by a different lysine (K) residue of the antibody. A linker comprises at least one covalent bond.

In some embodiments, complexes described herein comprise a structure of the formula (I): [R¹]ₙ₁—R², in which each R¹ independently comprises a compound comprising an oligonucleotide (e.g., a PMO) and R² comprises an antibody (e.g., an anti-TfR1 antibody), and wherein in each complex n1 is independently an integer (e.g., of one or greater) representing the number of instances of R¹ in each complex. In some embodiments, each R¹ independently comprises a group comprising an oligonucleotide. In some embodiments, each R¹ independently comprises a group that comprises additional elements in addition to an oligonucleotide. In some embodiments, R² comprises an antibody (e.g., an anti-TfR1 antibody) comprising a heavy chain comprising a heavy chain variable region (VH) and a heavy chain constant region, and a light chain comprising a light chain variable region (VL) and a light chain constant region. In some embodiments, each R¹ is covalently linked to R² via a linkage site represented by a lysine (K) residue of the antibody. In some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody. In some embodiments, each R¹ is covalently linked to R² via a linkage site represented by a lysine (K) residue of the antibody. In some embodiments, the linkage site is represented by the K at position 4 of a sequence motif DYEKHKVYA (SEQ ID NO: 53) of the light chain constant region of each antibody. In some embodiments, the linkage site is represented by the K at position 6 of a sequence motif DYEKHKVYA (SEQ ID NO: 53) of the light chain constant region of each antibody. In some embodiments, the linkage sites are represented by the K at position 4 and the K at position 6 of a sequence motif DYEKHKVYA (SEQ ID NO: 53) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to R¹ at linkage site represented by a lysine (K) residue in a sequence motif DYEKHKVYA (SEQ ID NO: 53) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody. In some embodiments, R² is an anti-TfR1 Fab.

In some embodiments, in each complex n1 is independently an integer of one or greater. In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)2 fragment, an scFv, or an Fv. In some embodiments, the antibody is a Fab fragment.

In some embodiments, in each complex n1 is independently an integer of one or greater. In some embodiments, the antibody comprises a sequence as set forth in Table 3. For example, in some embodiments, the antibody comprises a heavy chain complementarity determining region 1 (CDR-H1) comprising a sequence as set forth in SEQ ID NOs: 37, 43, or 48, a heavy chain complementarity determining region 2 (CDR-H2) comprising a sequence as set forth in SEQ ID NOs: 38, 44, or 49, a heavy chain complementarity determining region 3 (CDR-H3) comprising a sequence as set forth in SEQ ID NOs: 39, 45, or 50; and/or comprises a light chain complementarity determining region 1 (CDR-L1) comprising a sequence as set forth in SEQ ID NOs: 40, 46, or 51, a light chain complementarity determining region 2 (CDR-L2) comprising a sequence as set forth in SEQ ID NOs: 41 or 47, and a light chain complementarity determining region 3 (CDR-L3) comprising a sequence as set forth in SEQ ID NO: 42 or 52. In some embodiments, the antibody comprises a heavy chain variable region (VH) comprising an amino acid sequence at least 85% (e.g., at least 95%) identical to SEQ ID NO: 54 and/or comprises a light chain variable region (VL) comprising an amino acid sequence at least 85% (e.g., at least 95%) identical to SEQ ID NO: 55. In some embodiments, the antibody comprises a VH comprising the amino acid sequence of SEQ ID NO: 54 and/or comprises a VL comprising the amino acid sequence of SEQ ID NO: 55. In some embodiments, the antibody comprises a heavy chain comprising an amino acid sequence at least 85% (e.g., at least 95%) identical to SEQ ID NO: 56 and/or comprises a light chain comprising an amino acid sequence at least 85% (e.g., at least 95%) identical to SEQ ID NO: 57. In some embodiments, the antibody comprises a heavy chain comprising the amino acid sequence of SEQ ID NO: 56 and/or comprises a light chain comprising the amino acid sequence of SEQ ID NO: 57. In some embodiments, the antibody is a Fab fragment, a full-length IgG, a Fab′ fragment, a F(ab′)2 fragment, an scFv, or an Fv. In some embodiments, the antibody is a Fab fragment.

In some embodiments, the plurality of different complexes comprise a common targeting agent (e.g. an antibody) and a common oligonucleotide (e.g., PMO). In such embodiments, different complex types are characterized by having different numbers of oligonucleotides covalently linked to an antibody. For example, in some embodiments, a composition comprises a plurality of complex types in which each complex type comprises a structure of the formula (I): [R¹]ₙ₁—R², in which each R¹ independently comprises a compound comprising an oligonucleotide (e.g., a PMO) and R² comprises an antibody (e.g., anti-TfR1 antibody) comprising a heavy chain comprising a heavy chain variable region (VH) and a heavy chain constant region, and a light chain comprising a light chain variable region (VL) and a light chain constant region. In some embodiments, the value of n1 of each or any complex (e.g., any complex in any of the compositions or methods disclosed herein) is an integer from one up to the number of amino acid residues in the antibody to which conjugation is desired or targeted (e.g., the number of lysine residues). In some embodiments, in each complex the value of n1 is independently selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, in each complex the value of n1 is independently selected from 1, 2, 3, 4, 5, 6, 7, 8, 9, 10, 11, 12, 13, 14, 15, 16, 17, 18, 19, 20, 21, 22, 23, 24, 25 and 26. In some embodiments, in each complex the value of n1 is independently in the range of 1-27, 1-26, 1-10, 1-5, or 1-3. In some embodiments, the average value of n1 of complexes of the composition is in the range of 1 to 10 (e.g., 1-10, 1-5, 1-4, 1-3, 3-10, 3-5, or 5-10). In some embodiments, in each complex type n1 is independently an integer of one or greater representing the number of instances of R¹ in each complex of the complex type, and in which the different complex types of the composition are characterized by having different n1 values (e.g., n1 values in the range of 1-27, 1-26, 1-25, 1-20, 1-15, 1-10, 1-5, or 1-3).

In some embodiments, a composition described herein comprises unconjugated antibody (e.g., in trace amounts) and antibody conjugated to one or more oligonucleotides. In some embodiments, unconjugated antibody may be referred to as a compound of structure of the formula (I): [R¹]ₙ₁—R², for which n1 is zero. Accordingly, in some embodiments, a composition for administration to a subject in the methods described herein comprises compounds (e.g., complexes) of the structure of the formula (I): [R¹]ₙ₁—R², for which each R¹ independently comprises a group comprising an oligonucleotide, R² comprises an antibody and n1 is independently an integer of zero or greater that reflects the number of instances of R¹ in each compound (e.g., complex). In some embodiments, the fraction of compounds of the structure of the formula (I): [R¹]ₙ₁—R², in a composition, for which n1 is zero, compared with all compounds of that structure in the composition for which n1 is one or greater, is less than 10%, less than 5%, less than 1% less than 0.5%, less than 0.1%, less than 0.05%, or less than 0.01%.

In some embodiments, complexes described herein comprise a structure of the formula (I): [R¹]ₙ₁—R², in which each R¹ independently comprises a group of the formula (Ia):

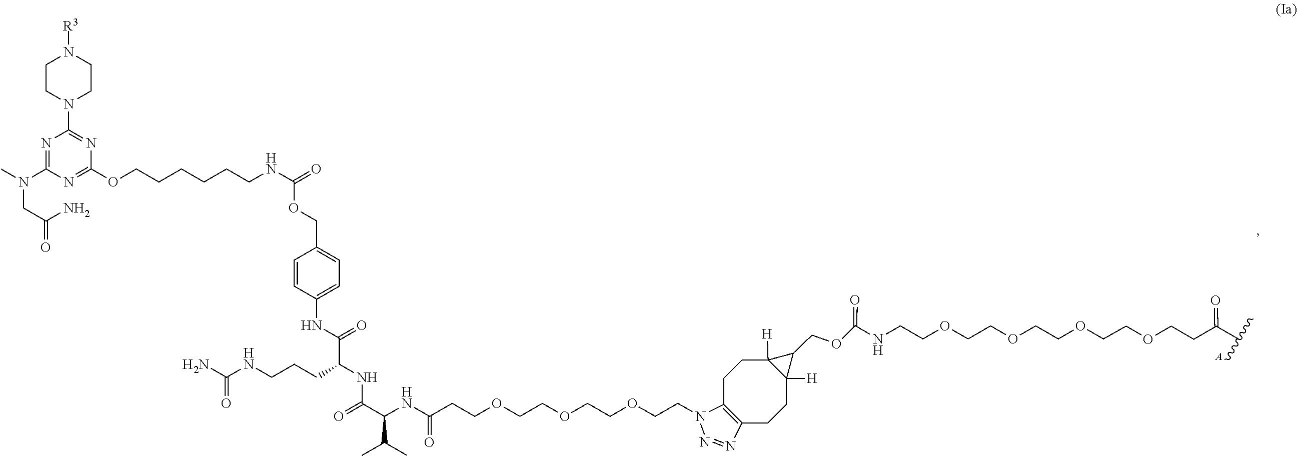

in which R³ is an oligonucleotide, e.g., a phosphorodiamidate morpholino oligomer (PMO); R² comprises an antibody comprising a heavy chain comprising a heavy chain variable region (VH) and a heavy chain constant region, and a light chain comprising a light chain variable region (VL) and a light chain constant region, wherein in each complex n1 is independently an integer (e.g., of one or greater) representing the number of instances of R¹ in each complex, and each R¹ is covalently linked to R² at attachment point A via a linkage site represented by a lysine (K) residue of the antibody. In some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, in a composition comprising a plurality of complexes described herein, at least 80% of the light chain constant regions of the antibodies of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, R² comprises an anti-TfR1 antibody (e.g., a Fab) comprising a sequence as set forth in Table 2. For example, in some embodiments, R² comprises an anti-TfR1 antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody (e.g., a Fab) comprising a VH comprising the amino acid sequence of SEQ ID NO: 17 and/or comprising a VL comprising the amino acid sequence of SEQ ID NO: 18. In some embodiments, R² comprises an anti-TfR1 antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody (e.g., a Fab) comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, R³ is an oligonucleotide, e.g., a phosphorodiamidate morpholino oligomer (PMO) comprising the base sequence of CTCCAACATCAAGGAAGATGGCATTTCTAG (SEQ ID NO: 21). In some embodiments, R² comprises a Fab and each R¹ is covalently linked at attachment point A to R² via a linkage site represented by a lysine (K) residue of the Fab, and wherein, in a composition comprising a plurality of complexes described herein, at least 80% (e.g., 80%-98%, 80%-95%, 80%-90%, 85%-98%, 85%-95%, 85%-90%, 90%-98%, or 90%-95%) of the light chain constant regions of the Fabs of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, in a composition comprising a plurality of complexes described herein, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody. In some embodiments, in each complex n1 is independently an integer (e.g., an integer in the range of 1-27, 1-26, 1-10, 1-5, or 1-3). In some embodiments, in a composition comprising a plurality of complexes described herein, the average value of n1 of complexes of the composition is in the range of 1 to 10 (e.g., 1-10, 1-5, 1-4, 1-3, 3-10, 3-5, or 5-10).

In some embodiments, complexes described herein comprise a structure of for the formula (I): [R¹]ₙ₁—R², in which each R¹ comprises a group of the formula (Ib):

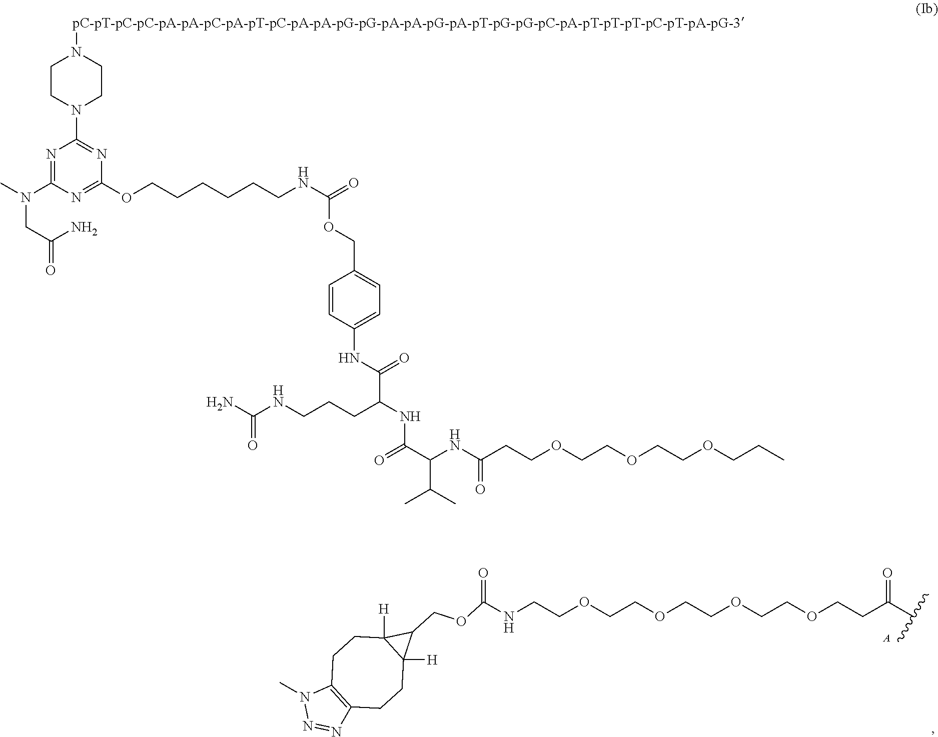

wherein -p is a phosphorodiamidate linkage of a phosphorodiamidate morpholino oligomer (PMO), and wherein the PMO comprises a base sequence of CTCCAACATCAAGGAAGATGGCATTTCTAG (SEQ ID NO: 21), R² comprises an antibody comprising a heavy chain comprising a heavy chain variable region (VH) and a heavy chain constant region, and a light chain comprising a light chain variable region (VL) and a light chain constant region, wherein in each complex n1 is independently an integer (e.g., of one or greater) representing the number of instances of R¹ in each complex, and each R¹ is covalently linked to R² at attachment point A via a linkage site represented by a lysine (K) residue of the antibody. In some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, in a composition comprising a plurality of complexes described herein, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, R² comprises an anti-TfR1 antibody (e.g., a Fab) comprising a sequence as set forth in Table 2. For example, in some embodiments, R² comprises an anti-TfR1 antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody (e.g., a Fab) comprising a VH comprising the amino acid sequence of SEQ ID NO: 17 and/or comprising a VL comprising the amino acid sequence of SEQ ID NO: 18. In some embodiments, R² comprises an anti-TfR1 antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody (e.g., a Fab) comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, R² comprises a Fab and each R¹ is covalently linked attachment point A to R² via a linkage site represented by a lysine (K) residue of the Fab, and wherein, in a composition comprising a plurality of complexes described herein, at least 80% (e.g., 80%-98%, 80%-95%, 80%-90%, 85%-98%, 85%-95%, 85%-90%, 90%-98%, or 90%-95%) of the light chain constant regions of the Fabs of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, in a composition comprising a plurality of complexes described herein, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody. In some embodiments, in a composition comprising a plurality of complexes described herein, the average value of n1 of complexes of the composition is in the range of 1 to 10 (e.g., 1-10, 1-5, 1-4, 1-3, 3-10, 3-5, or 5-10).

In some embodiments, complexes described herein comprise a structure of the formula (I): [R¹]ₙ₁—R², in which each R¹ comprises a group of the formula (Ic):

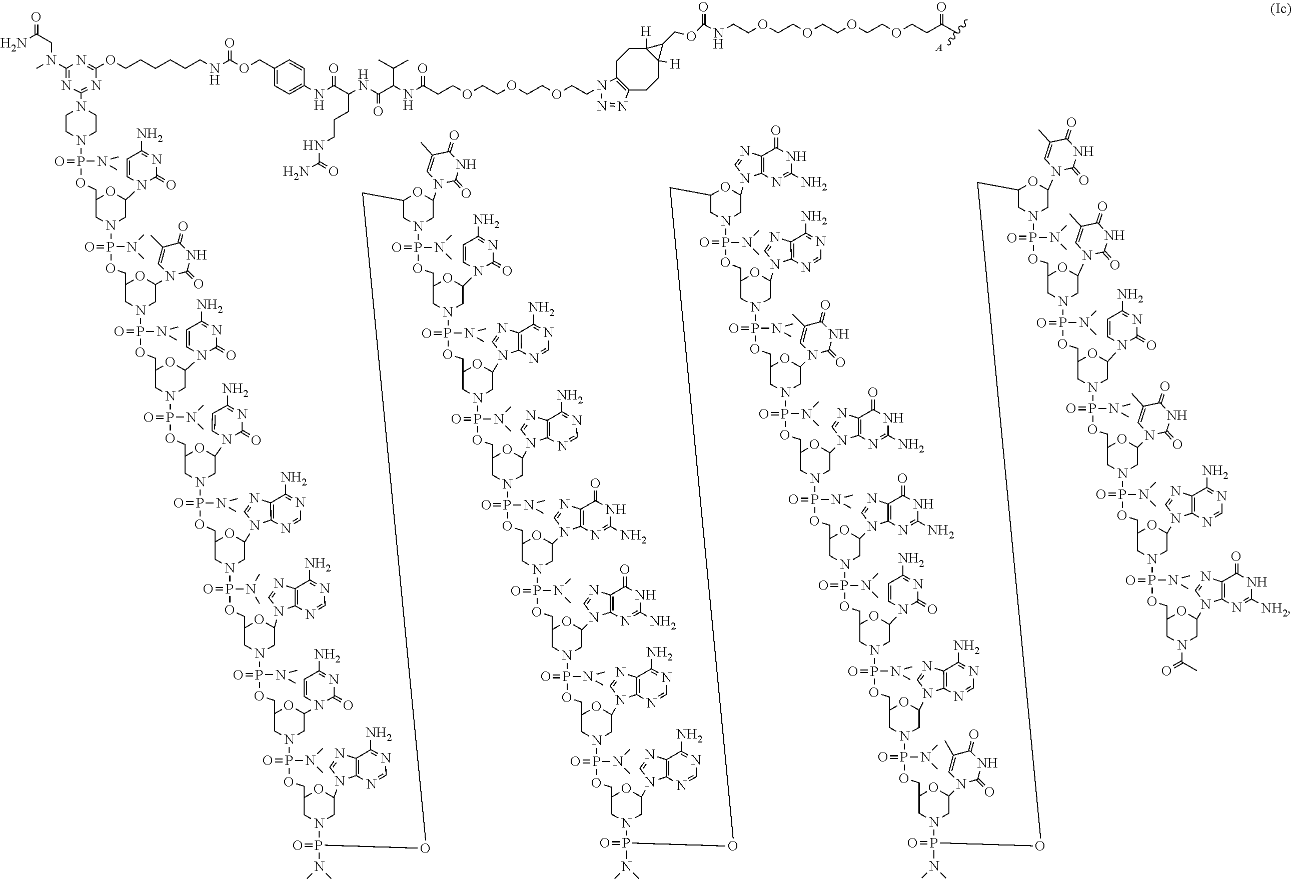

R² comprises an antibody comprising a heavy chain comprising a heavy chain variable region (VH) and a heavy chain constant region, and a light chain comprising a light chain variable region (VL) and a light chain constant region, wherein in each complex n1 is independently an integer (e.g., of one or greater) representing the number of instances of R¹ in each complex, wherein each R¹ is covalently linked to R² at attachment point A via a linkage site represented by a lysine (K) residue of the antibody. In some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, in a composition comprising a plurality of complexes described herein, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, R² comprises an anti-TfR1 antibody (e.g., a Fab) comprising a sequence as set forth in Table 2. For example, in some embodiments, R² comprises an anti-TfR1 antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody (e.g., a Fab) comprising a VH comprising the amino acid sequence of SEQ ID NO: 17 and/or comprising a VL comprising the amino acid sequence of SEQ ID NO: 18. In some embodiments, R² comprises an anti-TfR1 antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody (e.g., a Fab) comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, R² comprises a Fab and each R¹ is covalently linked at attachment point A to R² via a linkage site represented by a lysine (K) residue of the Fab, and wherein, in a composition comprising a plurality of complexes described herein, at least 80% (e.g., 80%-98%, 80%-95%, 80%-90%, 85%-98%, 85%-95%, 85%-90%, 90%-98%, or 90%-95%) of the light chain constant regions of the Fabs of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, in a composition comprising a plurality of complexes described herein, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody. In some embodiments, in a composition comprising a plurality of complexes described herein, the average value of n1 of complexes of the composition is in the range of 1 to 10 (e.g., 1-10, 1-5, 1-4, 1-3, 3-10, 3-5, or 5-10).

In some embodiments, complexes described herein comprise a structure of the formula (Id):

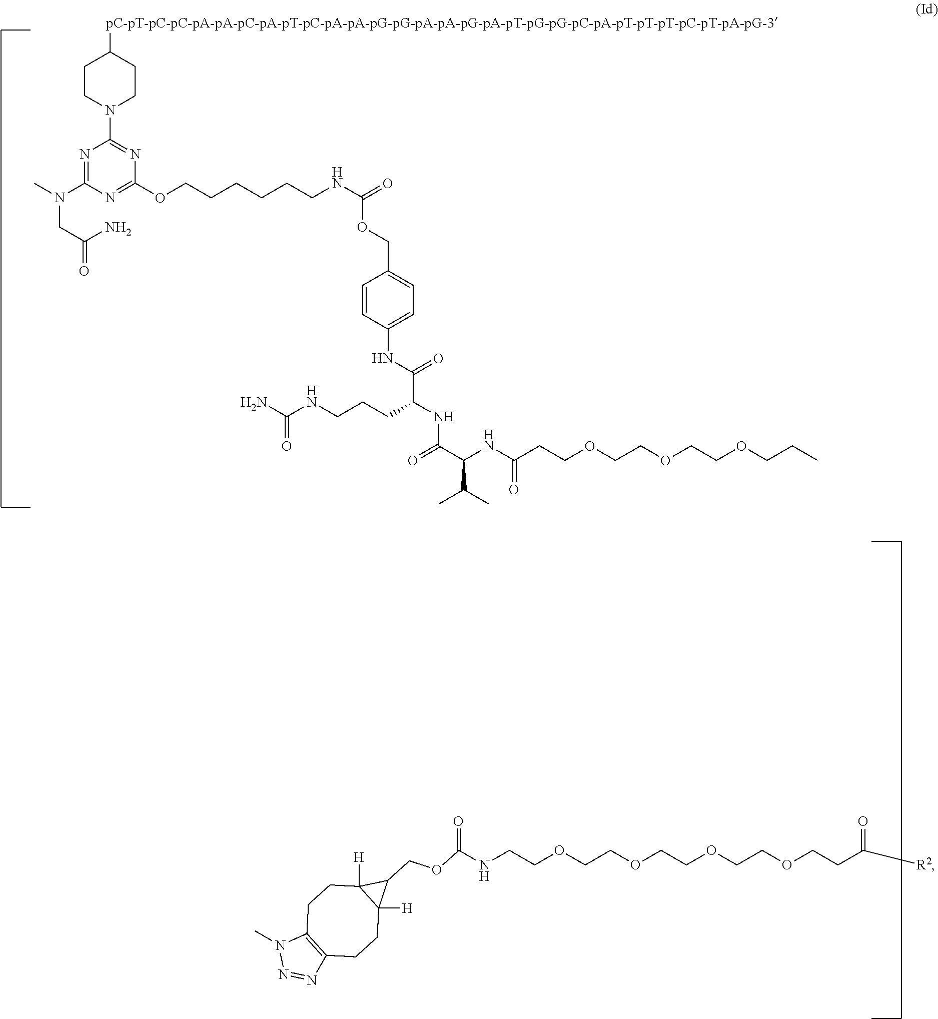

wherein -p is a phosphorodiamidate linkage of a phosphorodiamidate morpholino oligomer (PMO), and wherein the PMO comprises a base sequence of CTCCAACATCAAGGAAGATGGCATTTCTAG (SEQ ID NO: 21); wherein R² comprises an anti-TfR1 antibody (e.g., a Fab) comprising a CDR-H1, a CDR-H2, a CDR-H3, a CDR-L1, a CDR-L2, and a CDR-L3 selected from Table 2, optionally wherein the anti-TfR1 antibody (e.g., a Fab) comprises a VH comprising the amino acid sequence of SEQ ID NO: 17 and a VL comprising the amino acid sequence of SEQ ID NO: 18, further optionally wherein the anti-TfR1 antibody (e.g, a Fab) comprises a heavy chain comprising the amino acid sequence of SEQ ID NO: 19 and a light chain comprising the amino acid sequence of SEQ ID NO: 20; and wherein in each complex n1 is independently an integer (e.g., of one or greater) representing the number of instances of the group enclosed by square brackets, wherein each instance of the group enclosed by square brackets is covalently linked to a linkage site represented by a lysine (K) residue of the antibody (e.g., the Fab). In some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, in a composition comprising a plurality of complexes described herein, at least 80% (e.g., 80%-98%, 80%-95%, 80%-90%, 85%-98%, 85%-95%, 85%-90%, 90%-98%, or 90%-95%) of the light chain constant regions of the antibodies (e.g., the Fabs) of the complexes in the composition are independently covalently linked to R1 at a linkage site represented by K188 (based on Kabat numbering) and/or a linkage site represented by K190 (based on Kabat numbering) of the light chain constant region of each antibody. In some embodiments, R² comprises an antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody (e.g., a Fab) comprising a VH comprising the amino acid sequence of SEQ ID NO: 17 and/or comprising a VL comprising the amino acid sequence of SEQ ID NO: 18. In some embodiments, R² comprises an antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody (e.g., a Fab) comprising a heavy chain comprising the amino acid sequence of SEQ ID NO: 19 and/or comprising a light chain comprising the amino acid sequence of SEQ ID NO: 20. In some embodiments, in each complex n1 is independently an integer (e.g., an integer in the range of 1-27, 1-26, 1-10, 1-5, or 1-3). In some embodiments, R² comprises an antibody (e.g., a Fab) that is covalently linked via a linkage site represented by a lysine (K) residue of the antibody (e.g., the Fab), and wherein, in a composition comprising a plurality of complexes described herein, at least 80% (e.g., 80%-98%, 80%-95%, 80%-90%, 85%-98%, 85%-95%, 85%-90%, 90%-98%, or 90%-95%) of the light chain constant regions of the antibodies (e.g., the Fabs) of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, in a composition comprising a plurality of complexes described herein, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody. In some embodiments, in a composition comprising a plurality of complexes described herein, the average value of n1 of complexes of the composition is in the range of 1 to 10 (e.g., 1-10, 1-5, 1-4, 1-3, 3-10, 3-5, or 5-10).

In some embodiments, complexes described herein comprise a structure of:

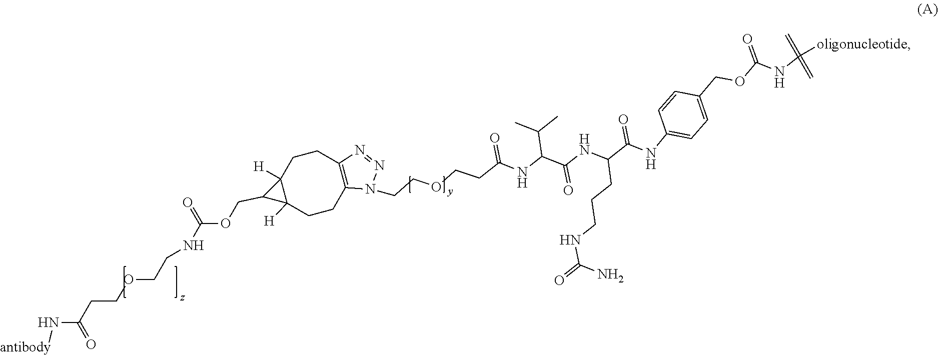

wherein y is 0-15 (e.g., 3) and z is 0-15 (e.g., 4). In some embodiments, the antibody is an an-TfR1 antibody (e.g., the anti-TfR1 antibody provided in Table 2) comprising a heavy chain comprising a heavy chain variable region (VH) and a heavy chain constant region, and a light chain comprising a light chain variable region (VL) and a light chain constant region of each antibody. In some embodiments, the oligonucleotide is a PMO and comprises the nucleotide sequence of SEQ ID NO: 21. In some embodiments, the amide shown adjacent to the anti-TfR1 antibody in the structure results from a reaction with an amine of the anti-TfR1 antibody, such as a lysine epsilon amine. In some embodiments, a complex described herein comprises an anti-TfR1 Fab covalently linked to the 5′ end of a PMO via a linkage site represented by a lysine (K) residue of the antibody. In some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, in a composition comprising a plurality of complexes described herein, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, in a composition comprising a plurality of complexes described herein, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody. In some embodiments, the anti-TfR1 antibody comprises a sequence as set forth in Table 2. For example, in some embodiments, the anti-TfR1 antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the anti-TfR1 antibody comprises a VH comprising the amino acid sequence of SEQ ID NO: 17 and/or comprises a VL comprising the amino acid sequence of SEQ ID NO: 18. In some embodiments, the anti-TfR1 antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the anti-TfR1 antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)2 fragment, an scFv, or an Fv.

In some embodiments, complexes described herein comprise a structure of the formula (I): [R₁]ₙ₁—R², in which each R¹ independently comprises a group of the formula (Ia):

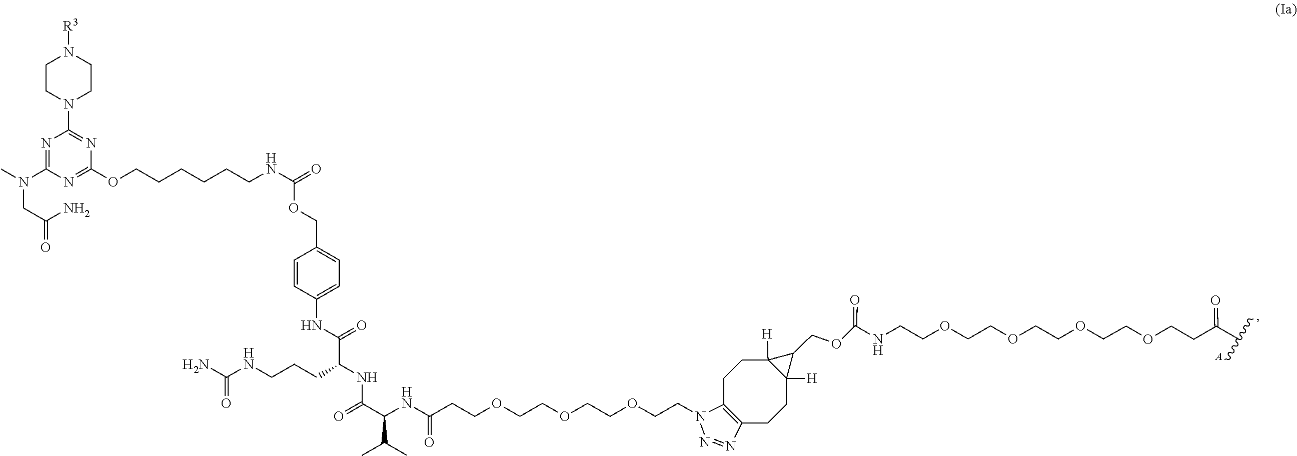

in which R³ is an oligonucleotide, e.g., a phosphorodiamidate morpholino oligomer (PMO); R² comprises an antibody comprising a heavy chain comprising a heavy chain variable region (VH) and a heavy chain constant region, and a light chain comprising a light chain variable region (VL) and a light chain constant region, wherein in each complex n1 is independently an integer (e.g., of one or greater) representing the number of instances of R¹ in each complex, and each R¹ is covalently linked to R² at attachment point A via a linkage site represented by a lysine (K) residue of the antibody. In some embodiments, the linkage site is represented by the K at position 4 of a sequence motif DYEKHKVYA (SEQ ID NO: 53) of the light chain constant region of each antibody. In some embodiments, the linkage site is represented by the K at position 6 of a sequence motif DYEKHKVYA (SEQ ID NO: 53) of the light chain constant region of each antibody. In some embodiments, the linkage sites are represented by the K at position 4 and the K at position 6 of a sequence motif DYEKHKVYA (SEQ ID NO: 53) of the light chain constant region of each antibody. In some embodiments, in a composition comprising a plurality of complexes described herein, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, R² comprises an anti-TfR1 antibody comprising a sequence as set forth in Table 2. For example, in some embodiments, R² comprises an anti-TfR1 antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody (e.g., a Fab) comprising a VH comprising the amino acid sequence of SEQ ID NO: 17 and/or comprising a VL comprising the amino acid sequence of SEQ ID NO: 18. In some embodiments, R² comprises an anti-TfR1 antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody (e.g., a Fab) comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, R³ is an oligonucleotide, e.g., a phosphorodiamidate morpholino oligomer (PMO) comprising the base sequence of CTCCAACATCAAGGAAGATGGCATTTCTAG (SEQ ID NO: 21). In some embodiments, R² comprises a Fab and each R¹ is covalently linked at attachment point A to R² via a linkage site represented by a lysine (K) residue of the Fab, and wherein, in a composition comprising a plurality of complexes described herein, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by a lysine (K) residues in a sequence motif DYEKHKVYA (SEQ ID NO: 53) of the light chain constant region of each antibody. In some embodiments, in a composition comprising a plurality of complexes described herein, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody. In some embodiments, in each complex n1 is independently an integer (e.g., an integer in the range of 1-27, 1-26, 1-10, 1-5, or 1-3). In some embodiments, in a composition comprising a plurality of complexes described herein, the average value of n1 of complexes of the composition is in the range of 1 to 10 (e.g., 1-10, 1-5, 1-4, 1-3, 3-10, 3-5, or 5-10).

In some embodiments, complexes described herein comprise a structure of the formula (I): [R¹]ₙ₁—R², in which each R¹ comprises a group of the formula (Ib):

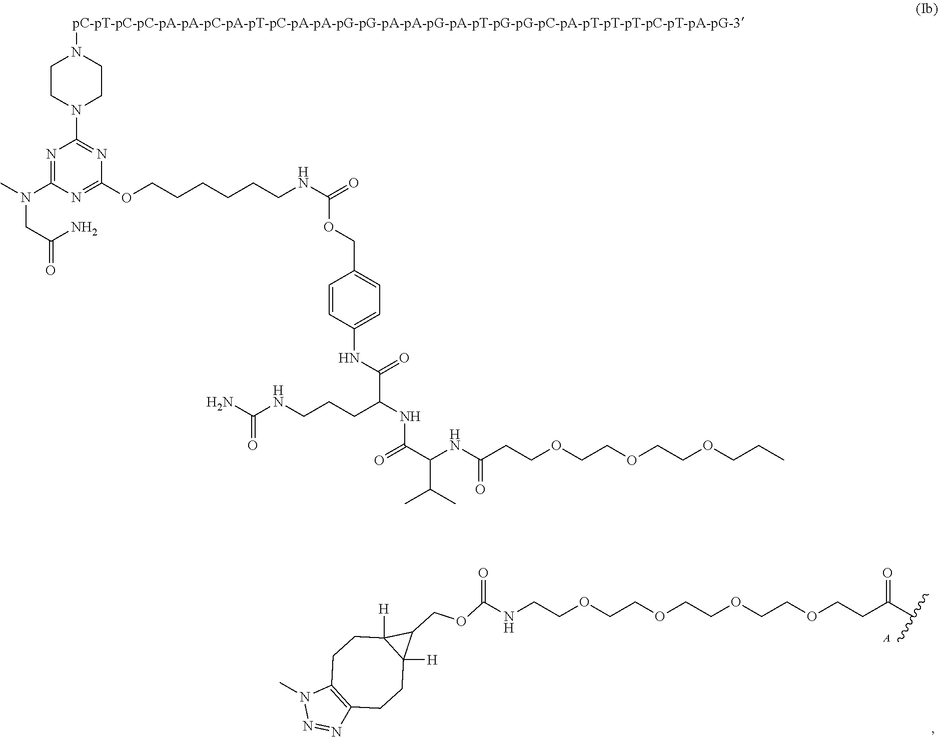

wherein -p is a phosphorodiamidate linkage, of a phosphorodiamidate morpholino oligomer (PMO), and wherein the PMO comprises a base sequence of CTCCAACATCAAGGAAGATGGCATTTCTAG (SEQ ID NO: 21), R² comprises an antibody comprising a heavy chain comprising a heavy chain variable region (VH) and a heavy chain constant region, and a light chain comprising a light chain variable region (VL) and a light chain constant region, wherein in each complex n1 is independently an integer (e.g., of one or greater) representing the number of instances of R¹ in each complex, and each R¹ is covalently linked to R² at attachment point A via a linkage site represented by a lysine (K) residue of the antibody. In some embodiments, the linkage site is represented by the K at position 4 of a sequence motif DYEKHKVYA (SEQ ID NO: 53) of the light chain constant region of each antibody. In some embodiments, the linkage site is represented by the K at position 6 of a sequence motif DYEKHKVYA (SEQ ID NO: 53) of the light chain constant region of each antibody. In some embodiments, the linkage sites are represented by the K at position 4 and the K at position 6 of a sequence motif DYEKHKVYA (SEQ ID NO: 53) of the light chain constant region of each antibody. In some embodiments, in a composition comprising a plurality of complexes described herein, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, R² comprises an anti-TfR1 antibody (e.g., a Fab) comprising a sequence as set forth in Table 2. For example, in some embodiments, R² comprises an anti-TfR1 antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody (e.g., a Fab) comprising a VH comprising the amino acid sequence of SEQ ID NO: 17 and/or comprising a VL comprising the amino acid sequence of SEQ ID NO: 18. In some embodiments, R² comprises an anti-TfR1 antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody(e.g., a Fab) comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, R² comprises a Fab and each R¹ is covalently linked at attachment point A to R² via a linkage site represented by a lysine (K) residue of the Fab, and wherein, in a composition comprising a plurality of complexes described herein, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, in a composition comprising a plurality of complexes described herein, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody. In some embodiments, in a composition comprising a plurality of complexes described herein, the average value of n1 of complexes of the composition is in the range of 1 to 10 (e.g., 1-10, 1-5, 1-4, 1-3, 3-10, 3-5, or 5-10).

In some embodiments, complexes described herein comprise a structure of the formula (I): [R¹]ₙ₁—R², in which each R¹ comprises a group of the formula (Ic):

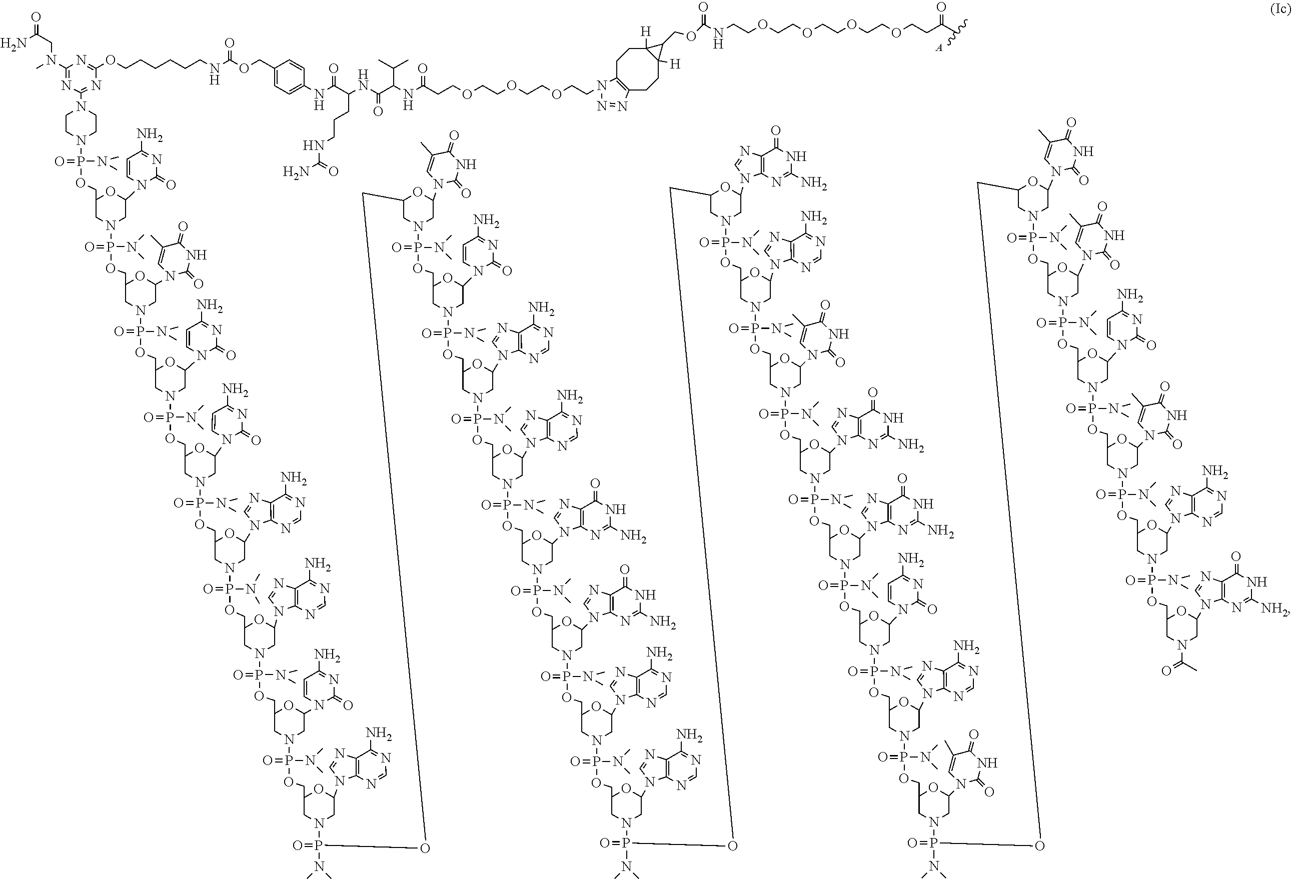

R² comprises an antibody comprising a heavy chain comprising a heavy chain variable region (VH) and a heavy chain constant region, and a light chain comprising a light chain variable region (VL) and a light chain constant region, wherein in each complex n1 is independently an integer (e.g., of one or greater) representing the number of instances of R¹ in each complex, wherein each R¹ is covalently linked to R² at attachment point A via a linkage site represented by a lysine (K) residue of the antibody. In some embodiments, the linkage site is represented by the K at position 4 of a sequence motif DYEKHKVYA (SEQ ID NO: 53) of the light chain constant region of each antibody. In some embodiments, the linkage site is represented by the K at position 6 of a sequence motif DYEKHKVYA (SEQ ID NO: 53) of the light chain constant region of each antibody. In some embodiments, the linkage sites are represented by the K at position 4 and the K at position 6 of a sequence motif DYEKHKVYA (SEQ ID NO: 53) of the light chain constant region of each antibody. In some embodiments, in a composition comprising a plurality of complexes described herein, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, R² comprises an anti-TfR1 antibody (e.g., a Fab) comprising a sequence as set forth in Table 2. For example, in some embodiments, R² comprises an anti-TfR1 antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody comprising a VH comprising the amino acid sequence of SEQ ID NO: 17 and/or comprising a VL comprising the amino acid sequence of SEQ ID NO: 18. In some embodiments, R² comprises an anti-TfR1 antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody (e.g., a Fab) comprising a heavy chain comprising the amino acid sequence of SEQ ID NO: 19 and/or comprising a light chain comprising the amino acid sequence of SEQ ID NO: 20. In some embodiments, R² comprises an antibody (e.g., a Fab) that is a Fab fragment, a full-length IgG, a Fab′ fragment, a F(ab′)2 fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, R² comprises a Fab and each R¹ is covalently linked at attachment point A to R² via a linkage site represented by a lysine (K) residue of the Fab, and wherein, in a composition comprising a plurality of complexes described herein, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, in a composition comprising a plurality of complexes described herein, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody. In some embodiments, in a composition comprising a plurality of complexes described herein, the average value of n1 of complexes of the composition is in the range of 1 to 10 (e.g., 1-10, 1-5, 1-4, 1-3, 3-10, 3-5, or 5-10).

In some embodiments, complexes described herein comprise a structure of formula (Id):

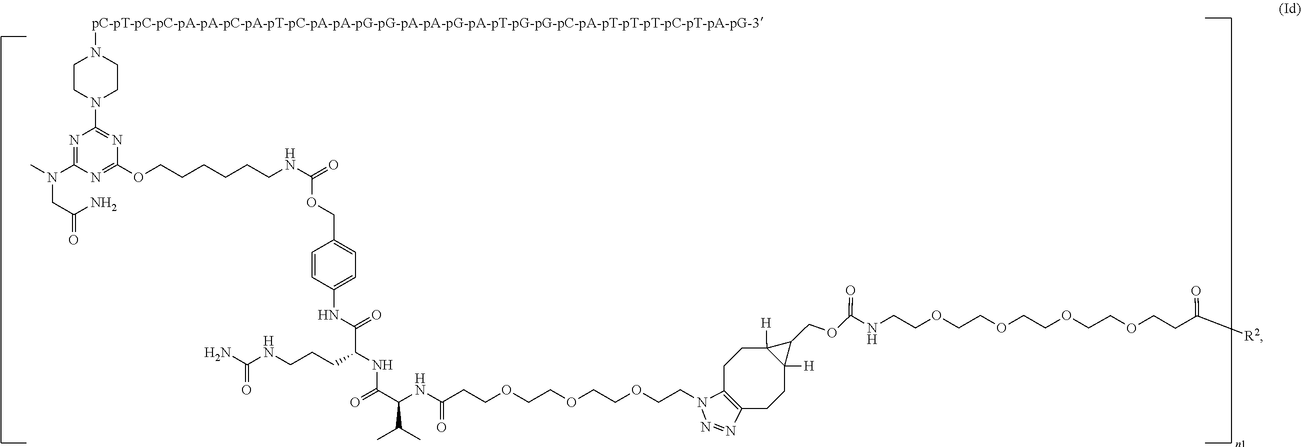

wherein -p is a phosphorodiamidate linkage of a phosphorodiamidate morpholino oligomer (PMO), and wherein the PMO comprises a base sequence of CTCCAACATCAAGGAAGATGGCATTTCTAG (SEQ ID NO: 21); wherein R² comprises an anti-TfR1 antibody (e.g., a Fab) comprising a CDR-H1, a CDR-H2, a CDR-H3, a CDR-L1, a CDR-L2, and a CDR-L3 selected from Table 2, optionally wherein the anti-TfR1 antibody (e.g., a Fab) comprises a VH comprising the amino acid sequence of SEQ ID NO: 17 and a VL comprising the amino acid sequence of SEQ ID NO: 18, further optionally wherein the anti-TfR1 antibody (e.g., a Fab) comprises a heavy chain comprising the amino acid sequence of SEQ ID NO: 19 and a light chain comprising the amino acid sequence of SEQ ID NO: 20; and wherein in each complex n1 is independently an integer (e.g., of one or greater) representing the number of instances of the group enclosed by square brackets, wherein each instance of the group enclosed by square brackets is covalently linked to a linkage site represented by a lysine (K) residue of the antibody (e.g., the Fab). In some embodiments, the linkage site is represented by the K at position 4 of a sequence motif DYEKHKVYA (SEQ ID NO: 53) of the light chain constant region of each antibody. In some embodiments, the linkage site is represented by the K at position 6 of a sequence motif DYEKHKVYA (SEQ ID NO: 53) of the light chain constant region of each antibody. In some embodiments, the linkage sites are represented by the K at position 4 and the K at position 6 of a sequence motif DYEKHKVYA (SEQ ID NO: 53) of the light chain constant region of each antibody. In some embodiments, in a composition comprising a plurality of complexes described herein, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, R² comprises an antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody (e.g., a Fab) comprising a VH comprising the amino acid sequence of SEQ ID NO: 17 and/or comprising a VL comprising the amino acid sequence of SEQ ID NO: 18. In some embodiments, R² comprises an antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody (e.g., a Fab) comprising a heavy chain comprising the amino acid sequence of SEQ ID NO: 19 and/or comprising a light chain comprising the amino acid sequence of SEQ ID NO: 20. In some embodiments, in each complex n1 is independently an integer (e.g., an integer in the range of 1-27, 1-26, 1-10, 1-5, or 1-3). In some embodiments, R² comprises an antibody (e.g., a Fab) that is covalently linked via a linkage site represented by a lysine (K) residue of the antibody (e.g., the Fab), and wherein, in a composition comprising a plurality of complexes described herein, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, in a composition comprising a plurality of complexes described herein, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody. In some embodiments, in a composition comprising a plurality of complexes described herein, the average value of n1 of complexes of the composition is in the range of 1 to 10 (e.g., 1-10, 1-5, 1-4, 1-3, 3-10, 3-5, or 5-10).

In some embodiments, complexes described herein comprise a structure of:

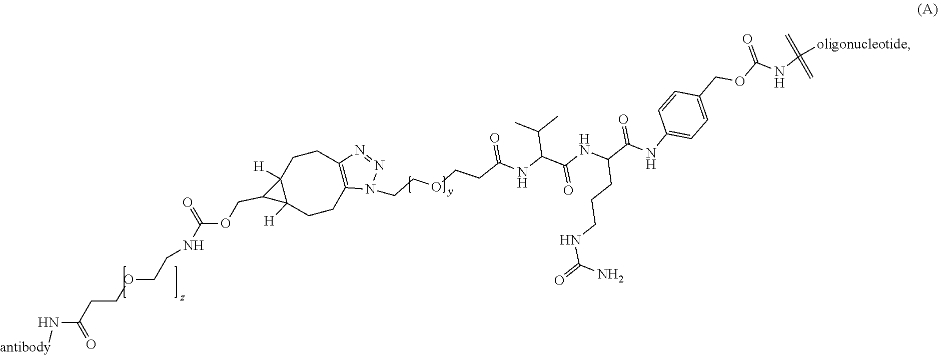

wherein y is 0-15 (e.g., 3) and z is 0-15 (e.g., 4). In some embodiments, the antibody is an an-TfR1 antibody (e.g., the anti-TfR1 antibody provided in Table 2) comprising a heavy chain comprising a heavy chain variable region (VH) and a heavy chain constant region, and a light chain comprising a light chain variable region (VL) and a light chain constant region. In some embodiments, the oligonucleotide is a PMO and comprises the nucleotide sequence of SEQ ID NO: 21. In some embodiments, the amide shown adjacent to the anti-TfR1 antibody in the structure results from a reaction with an amine of the anti-TfR1 antibody, such as a lysine epsilon amine. In some embodiments, a complex described herein comprises an anti-TfR1 Fab covalently linked to the 5′ end of a PMO via a linkage site represented by a lysine (K) residue of the antibody. In some embodiments, the linkage site is represented by the K at position 4 of a sequence motif DYEKHKVYA (SEQ ID NO: 53) of the light chain constant region of each antibody. In some embodiments, the linkage site is represented by the K at position 6 of a sequence motif DYEKHKVYA (SEQ ID NO: 53) of the light chain constant region of each antibody. In some embodiments, the linkage sites are represented by the K at position 4 and the K at position 6 of a sequence motif DYEKHKVYA (SEQ ID NO: 53) of the light chain constant region of each antibody. In some embodiments, in a composition comprising a plurality of complexes described herein, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, in a composition comprising a plurality of complexes described herein, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody. In some embodiments, the anti-TfR1 antibody comprises a sequence as set forth in Table 2. For example, in some embodiments, the anti-TfR1 antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the anti-TfR1 antibody comprises a VH comprising the amino acid sequence of SEQ ID NO: 17 and/or comprises a VL comprising the amino acid sequence of SEQ ID NO: 18. In some embodiments, the anti-TfR1 antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the anti-TfR1 antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)2 fragment, an scFv, or an Fv.

In each complex disclosed (e.g., comprising a structure of formula (I): [R¹]ₙ₁—R², such as a complex in which each R¹ comprises a group of the formula (Ia), (Ib), or (Ic); a complex comprising a structure of formula (Id); or a complex comprising a structure of formula (A)) may comprise a structure having the stereochemistry shown in formula (B):

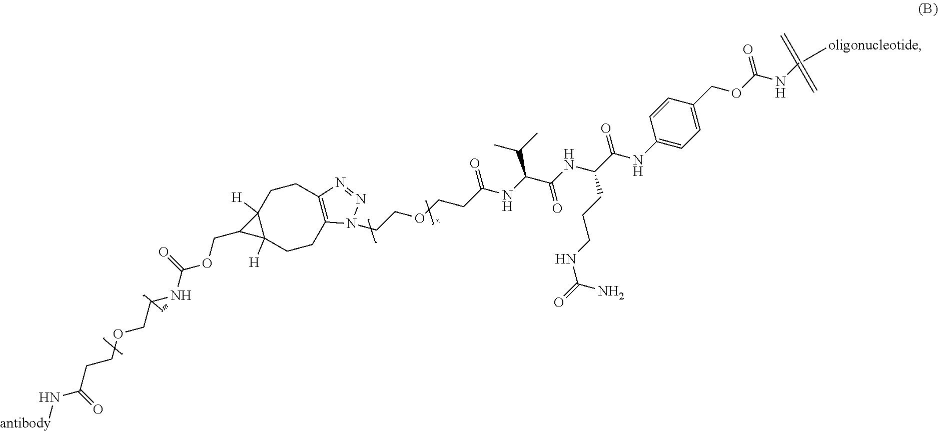

(B), wherein y is 0-15 (e.g., 3) and z is 0-15 (e.g., 4). It should be understood that the stereochemistry shown in formula (B) can be applied to the corresponding portion of any formula or structure provided herein (e.g., formula (Ia), (Ib), (Ic), (Id), or (A)).

### Linkage Sites

Provided herein are compositions comprising a plurality of complexes, wherein each complex comprises an antibody covalently linked to one or more oligonucleotides. In some embodiments, the antibody comprises a heavy chain comprising a heavy chain variable region (VH) and a heavy chain constant region, and a light chain comprising a light chain variable region (VL) and a light chain constant region. In some embodiments, each oligonucleotide is covalently linked at a linkage site represented by a lysine (K) residue of the antibody. In some embodiments, in each complex of the composition, each oligonucleotide is covalently linked at a linkage site represented by a different lysine (K) residue of the antibody.

In some embodiments, a linkage site is in the light chain of the antibody. In some embodiments, a linkage site is in the light chain constant region of the antibody. For example, in some embodiments, a linkage site is represented by K126, K145, K149, K188, K190, or K207 of the light chain constant region based on Kabat numbering. In some embodiments, a linkage site is in the light chain variable region of the antibody. For example, in some embodiments, a linkage site is represented by K39 or K107 of the light chain variable region based on Kabat numbering. In some embodiments, a linkage site is in the heavy chain constant region of the antibody. For example, in some embodiments, a linkage site is represented by K117, K213, K218, K221, K222, or K228 of the heavy chain constant region based on Kabat numbering. In some embodiments, a linkage site is in the heavy chain of the antibody. In some embodiments, a linkage site is in the heavy chain variable region of the antibody. For example, in some embodiments, a linkage site is represented by K13, K43, K64, or K81 of the heavy chain variable region based on Kabat numbering.

In some embodiments, the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. For example, in some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, at least 80% (e.g., at least 80%, at least 81%, at least 82%, at least 83%, at least 84%, at least 85% at least 86%, at least 87%, at least 88%, at least 89%, at least 90%, at least 91%, at least 92%, at least 93%, at least 94%, at least 95%, a least 96%, at least 97%, at least 98%, or at least 99%) of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. In some embodiments, about 80%-98%, 80%-95%, 80%-90%, 80%-85%, 85%-98%, 85%-95%, 85%-90%, 90%-98%, 90%-95%, or 95%-98% of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. In some embodiments, about 85%-95% (e.g., 85%-95%, 85%-90%, or 90%-95%) of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. In some embodiments, 90%-95% (e.g. about 90%, about 91%, about 92%, about 93%, about 94%, or about 95%) of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. It is to be understood that, complexes comprising light chain constant regions of the antibodies covalently linked to an oligonucleotide at a linkage site represented by K188 (based on Kabat numbering) and/or a linkage site represented by K190 (based on Kabat numbering) of the light chain constant region includes: complexes that are covalently linked to an oligonucleotide at a linkage site represented by K188 (based on Kabat numbering) of the light chain constant region; complexes that are covalently linked to an oligonucleotide at a linkage site represented by K190 (based on Kabat numbering) of the light chain constant region; and/or complexes that are covalently linked to an oligonucleotide at a linkage site represented by K188 (based on Kabat numbering) and at a linkage site represented by K190 (based on Kabat numbering) of the light chain constant region.

In some embodiments, the heavy chain constant regions of the antibodies of the complexes in the composition are independently covalently linked to an oligonucleotide at a linkage site represented by K213 (based on Kabat numbering) of the heavy chain constant region of each antibody. In some embodiments, at least 15% (e.g. at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, or at least 45%) of the heavy chain constant regions of the antibodies of the complexes in the composition are independently covalently linked to an oligonucleotide at a linkage site represented by K213 (based on Kabat numbering) of the heavy chain constant region of each antibody. In some embodiments, about 15%-45% (e.g., 15%-45%, 15%-40%, 15%-30%, 15%-20%, 20%-45% 20%-40%, 20%-30%, 30%-45% 30%-40%, or 40%-45%) of the heavy chain constant regions of the antibodies of the complexes in the composition are independently covalently linked to an oligonucleotide at a linkage site represented by K213 (based on Kabat numbering) of the heavy chain constant region of each antibody. For example, in some embodiments, about 15%-45%, 15%-40%, 15%-35%, 15%-30%, 15%-25%, 15%-20%, 20%-45%, 20%-40%, 20%-35%, 20%-30%, 20%-25%, 25%-45%, 25%-40%, 25%-35%, 25%-30%, 30%-45%, 30%-40%, 30%-35%, 35%-45%, 35%-40%, or 40%-45% of the heavy chain constant regions of the antibodies of the complexes in the composition are independently covalently linked to an oligonucleotide at a linkage site represented by K213 (based on Kabat numbering) of the heavy chain constant region of each antibody. In some embodiments, about 15%-30% (e.g. about 15%, about 16%, about 17%, about 18%, about 19%, about 20%, about 21%, about 22%, about 23%, about 24%, about 25%, about 26%, about 27%, about 28%, about 29%, or about 30%) of the heavy chain constant regions of the antibodies of the complexes in the composition are independently covalently linked to an oligonucleotide at a linkage site represented by K213 (based on Kabat numbering) of the heavy chain constant region of each antibody.

In some embodiments, the heavy chain constant regions of the antibodies of the complexes in the composition are independently covalently linked to an oligonucleotide at a linkage site represented by K221 (based on Kabat numbering) and/or a linkage site represented by K222 (based on Kabat numbering) of the heavy chain constant region of each antibody. For example, in some embodiments, the linkage site is represented by K221 (based on Kabat numbering) of the heavy chain constant region of each antibody. In some embodiments, the linkage site is represented by K222 (based on Kabat numbering) of the heavy chain constant region of each antibody. In some embodiments, the linkage sites are represented by K221 (based on Kabat numbering) and K222 (based on Kabat numbering) of the heavy chain constant region of each antibody. In some embodiments, at least 5% (e.g. at least 5%, at least 6%, at least 7%, at least 8%, at least 9%, at least 10%, at least 11%, at least 12%, at least 13%, at least 14%, at least 15%) of the heavy chain constant regions of the antibodies of the complexes in the composition are independently covalently linked to an oligonucleotide at a linkage site represented by K221 (based on Kabat numbering) and/or a linkage site represented by K222 (based on Kabat numbering) of the heavy chain constant region of each antibody. In some embodiments, 5%-15% (about 5%, about 6%, about 7%, about 8%, about 9%, about 10%, about 11%, about 12%, about 13%, about 14%, or about 15%) of the heavy chain constant regions of the antibodies of the complexes in the composition are independently covalently linked to an oligonucleotide at a linkage site represented by K221 (based on Kabat numbering) and/or a linkage site represented by K222 (based on Kabat numbering) of the heavy chain constant region of each antibody.

In some embodiments, the heavy chain variable regions of the antibodies of the complexes in the composition are independently covalently linked to an oligonucleotide at a linkage site represented by K13 (based on Kabat numbering) of the heavy chain variable region of each antibody. In some embodiments, at least 2% (e.g., at least 2%, at least 3%, at least 4%, at least 5%, at least 6%, at least 7%, at least 8%, at least 9%, or at least 10%) of the heavy chain variable regions of the antibodies of the complexes in the composition are independently covalently linked to an oligonucleotide at a linkage site represented by K13 (based on Kabat numbering) of the heavy chain variable region of each antibody. In some embodiments, 2%-10% (about 2%, about 3%, about 4%, about 5%, about 6%, about 7%, about 8%, about 9%, or about 10%) of the heavy chain variable regions of the antibodies of the complexes in the composition are independently covalently linked to an oligonucleotide at a linkage site represented by K13 (based on Kabat numbering) of the heavy chain variable region of each antibody.

In some embodiments, the heavy chain variable regions of the antibodies of the complexes in the composition are independently covalently linked to an oligonucleotide at a linkage site represented by K43 (based on Kabat numbering) of the heavy chain variable region of each antibody. In some embodiments, at least 2% (e.g., at least 2%, at least 3%, at least 4%, at least 5%, at least 6%, at least 7%, at least 8%, at least 9%, or at least 10%) of the heavy chain variable regions of the antibodies of the complexes in the composition are independently covalently linked to an oligonucleotide at a linkage site represented by K43 (based on Kabat numbering) of the heavy chain variable region of each antibody. In some embodiments, 2%-10% (about 2%, about 3%, about 4%, about 5%, about 6%, about 7%, about 8%, about 9%, or about 10%) of the heavy chain variable regions of the antibodies of the complexes in the composition are independently covalently linked to an oligonucleotide at a linkage site represented by K43 (based on Kabat numbering) of the heavy chain variable region of each antibody.

In some embodiments, the heavy chain variable regions of the antibodies of the complexes in the composition are independently covalently linked to an oligonucleotide at a linkage site represented by K81 (based on Kabat numbering) of the heavy chain variable region of each antibody. In some embodiments, at least 2% (e.g., at least 2%, at least 3%, at least 4%, at least 5%, at least 6%, at least 7%, at least 8%, at least 9%, or at least 10%) of the heavy chain variable regions of the antibodies of the complexes in the composition are independently covalently linked to an oligonucleotide at a linkage site represented by K81 (based on Kabat numbering) of the heavy chain variable region of each antibody. In some embodiments, 2%-10% (about 2%, about 3%, about 4%, about 5%, about 6%, about 7%, about 8%, about 9%, or about 10%) of the heavy chain variable regions of the antibodies of the complexes in the composition are independently covalently linked to an oligonucleotide at a linkage site represented by K81 (based on Kabat numbering) of the heavy chain variable region of each antibody.

In some embodiments, the light chain constant regions of the antibodies of the complexes in the composition are independently covalently linked to an oligonucleotide at a linkage site represented by K126 (based on Kabat numbering) of the light chain constant region of each antibody. In some embodiments, at least 2% (e.g., at least 2%, at least 3%, at least 4%, at least 5%, at least 6%, at least 7%, at least 8%, at least 9%, or at least 10%) of the light chain constant regions of the antibodies of the complexes in the composition are independently covalently linked to an oligonucleotide at a linkage site represented by K126 (based on Kabat numbering) of the light chain constant region of each antibody. In some embodiments, 2%-10% (about 2%, about 3%, about 4%, about 5%, about 6%, about 7%, about 8%, about 9%, or about 10%) of the light chain constant regions of the antibodies of the complexes in the composition are independently covalently linked to an oligonucleotide at a linkage site represented by K126 (based on Kabat numbering) of the light chain constant region of each antibody.

In some embodiments, the light chain constant regions of the antibodies of the complexes in the composition are independently covalently linked to an oligonucleotide at a linkage site represented by K145 (based on Kabat numbering) of the light chain constant region of each antibody. In some embodiments, at least 2% (e.g., at least 2%, at least 3%, at least 4%, at least 5%, at least 6%, at least 7%, at least 8%, at least 9%, or at least 10%) of the light chain constant regions of the antibodies of the complexes in the composition are independently covalently linked to an oligonucleotide at a linkage site represented by K145 (based on Kabat numbering) of the light chain constant region of each antibody. In some embodiments, 2%-10% (about 2%, about 3%, about 4%, about 5%, about 6%, about 7%, about 8%, about 9%, or about 10%) of the light chain constant regions of the antibodies of the complexes in the composition are independently covalently linked to an oligonucleotide at a linkage site represented by K145 (based on Kabat numbering) of the light chain constant region of each antibody.

In some embodiments, the light chain constant regions of the antibodies of the complexes in the composition are independently covalently linked to an oligonucleotide at a linkage site represented by K149 (based on Kabat numbering) of the light chain constant region of each antibody. In some embodiments, at least 2% (e.g., at least 2%, at least 3%, at least 4%, at least 5%, at least 6%, at least 7%, at least 8%, at least 9%, or at least 10%) of the light chain constant regions of the antibodies of the complexes in the composition are independently covalently linked to an oligonucleotide at a linkage site represented by K149 (based on Kabat numbering) of the light chain constant region of each antibody. In some embodiments, 2%-10% (about 2%, about 3%, about 4%, about 5%, about 6%, about 7%, about 8%, about 9%, or about 10%) of the light chain constant regions of the antibodies of the complexes in the composition are independently covalently linked to an oligonucleotide at a linkage site represented by K149 (based on Kabat numbering) of the light chain constant region of each antibody.

In some embodiments, the light chain constant regions of the antibodies of the complexes in the composition are independently covalently linked to an oligonucleotide at a linkage site represented by K207 (based on Kabat numbering) of the light chain constant region of each antibody. In some embodiments, at least 2% (e.g., at least 2%, at least 3%, at least 4%, at least 5%, at least 6%, at least 7%, at least 8%, at least 9%, or at least 10%) of the light chain constant regions of the antibodies of the complexes in the composition are independently covalently linked to an oligonucleotide at a linkage site represented by K207 (based on Kabat numbering) of the light chain constant region of each antibody. In some embodiments, 2%-10% (about 2%, about 3%, about 4%, about 5%, about 6%, about 7%, about 8%, about 9%, or about 10%) of the light chain constant regions of the antibodies of the complexes in the composition are independently covalently linked to an oligonucleotide at a linkage site represented by K207 (based on Kabat numbering) of the light chain constant region of each antibody.

In some embodiments, the light chain variable regions of the antibodies of the complexes in the composition are independently covalently linked to an oligonucleotide at a linkage site represented by K39 (based on Kabat numbering) of the light chain variable region of each antibody. In some embodiments, at least 2% (e.g., at least 2%, at least 3%, at least 4%, at least 5%, at least 6%, at least 7%, at least 8%, at least 9%, or at least 10%) of the light chain variable regions of the antibodies of the complexes in the composition are independently covalently linked to an oligonucleotide at a linkage site represented by K39 (based on Kabat numbering) of the light chain variable region of each antibody. In some embodiments, 2%-10% (about 2%, about 3%, about 4%, about 5%, about 6%, about 7%, about 8%, about 9%, or about 10%) of the light chain variable regions of the antibodies of the complexes in the composition are independently covalently linked to an oligonucleotide at a linkage site represented by K39 (based on Kabat numbering) of the light chain variable region of each antibody.

In some embodiments, the light chain variable regions of the antibodies of the complexes in the composition are independently covalently linked to an oligonucleotide at a linkage site represented by K107 (based on Kabat numbering) of the light chain variable region of each antibody. In some embodiments, at least 2% (e.g., at least 2%, at least 3%, at least 4%, at least 5%, at least 6%, at least 7%, at least 8%, at least 9%, or at least 10%) of the light chain variable regions of the antibodies of the complexes in the composition are independently covalently linked to an oligonucleotide at a linkage site represented by K107 (based on Kabat numbering) of the light chain variable region of each antibody. In some embodiments, 2%-10% (about 2%, about 3%, about 4%, about 5%, about 6%, about 7%, about 8%, about 9%, or about 10%) of the light chain variable regions of the antibodies of the complexes in the composition are independently covalently linked to an oligonucleotide at a linkage site represented by K107 (based on Kabat numbering) of the light chain variable region of each antibody.

In some embodiments, the heavy chain constant regions of the antibodies of the complexes in the composition are independently covalently linked to an oligonucleotide at a linkage site represented by K117 (based on Kabat numbering) of the heavy chain constant region of each antibody.

In some embodiments, the heavy chain constant regions of the antibodies of the complexes in the composition are independently covalently linked to an oligonucleotide at a linkage site represented by K218 (based on Kabat numbering) of the heavy chain constant region of each antibody.

In some embodiments, the heavy chain constant regions of the antibodies of the complexes in the composition are independently covalently linked to an oligonucleotide at a linkage site represented by K228 (based on Kabat numbering) of the heavy chain constant region of each antibody.

In some embodiments, the heavy chain variable regions of the antibodies of the complexes in the composition are independently covalently linked to an oligonucleotide at a linkage site represented by K64 (based on Kabat numbering) of the heavy chain variable region of each antibody.

In some embodiments, lysine (K) residue numbers referred to herein are based on Kabat numbering (Kabat et al. (1971) Ann. NY Acad, Sci. 190:382-391 and Kabat et al., Sequences of Proteins of Immunological Interest (National Institutes of Health, Bethesda, Md. (1987) and (1991)). The variable regions and constant regions of the heavy and light chains of the antibodies provided herein are numbered separately. Kabat numbering of the light chain variable regions and heavy chain variable regions of the antibodies are described in the art, e.g., in Kabat et al. (1971) Ann. NY Acad, Sci. 190:382-391 and Kabat et al., Sequences of Proteins of Immunological Interest (National Institutes of Health, Bethesda, Md. (1987) and (1991). Kabat numbering of the light chain constant regions and heavy chain constant regions of the antibodies provided herein can be found at imgt.org/IMGTScientificChart/Numbering/Hu_IGHGnber.html and imgt.org/IMGTScientificChart/Numbering/Hu_IGKCnber.html (also see Edelman, G. M. et al., Proc. Natl. Acad. USA, 63, 78-85 (1969). PMID: 5257969; Hieter, P. A. et al., Cell, 22, 197-207 (1980). PMID: 6775818; Sequences of Proteins of Immunological Interest (National Institutes of Health, Bethesda, Md. 647, 662, 680, 689 (1991)).

In some embodiments, the light chain constant regions of the antibodies of the complexes in the composition are independently covalently linked to an oligonucleotide at linkage sites represented by represented by lysine (K) residues in a sequence motif DYEKHKVYA (SEQ ID NO: 53) of the light chain constant region of each antibody. For example, in some embodiments, the linkage site is represented by the K at position 4 of a sequence motif DYEKHKVYA (SEQ ID NO: 53) of the light chain constant region of each antibody. In some embodiments, the linkage site is represented by the K at position 6 of a sequence motif DYEKHKVYA (SEQ ID NO: 53) of the light chain constant region of each antibody. In some embodiments, the linkage sites are represented by the K at position 4 and the K at position 6 of a sequence motif DYEKHKVYA (SEQ ID NO: 53) of the light chain constant region of each antibody. In some embodiments, at least 80% (e.g., at least 80%, at least 81%, at least 82%, at least 83%, at least 84%, at least 85% at least 86%, at least 87%, at least 88%, at least 89%, at least 90%, at least 91%, at least 92%, at least 93%, at least 94%, at least 95%, a least 96%, at least 97%, at least 98%, or at least 99%) of the light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, 80%-98%, 80%-95%, 80%-90%, 80%-85%, 85%-98%, 85%-95, 85%-90%, 90%-98%, 90%-95%, or 95%-98% of the light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, 85%-95% (e.g., 85%-95%, 85%-90%, or 90%-95%) of the light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, 90%-95% (e.g., about 90%, about 91%, about 92%, about 93%, about 94%, or about 95%) of the light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. It is to be understood that, complexes comprising light chain constant regions of the antibodies covalently linked to an oligonucleotide at a linkage site represented by lysine (K) residues in a sequence motif DYEKHKVYA (SEQ ID NO: 53) of the light chain constant region includes: complexes that are covalently linked to an oligonucleotide at a linkage site represented by the K at position 4 in a sequence motif DYEKHKVYA (SEQ ID NO: 53) of the light chain constant region; complexes that are covalently linked to an oligonucleotide at a linkage site represented by the K at position 6 in a sequence motif DYEKHKVYA (SEQ ID NO: 53) of the light chain constant region; and/or complexes that are covalently linked to an oligonucleotide at a linkage site represented the K at position 4 and at a linkage site represented by the K at position 6 in a sequence motif DYEKHKVYA (SEQ ID NO: 53) of the light chain constant region.

In some embodiments, the heavy chain constant regions of the antibodies of the complexes in the composition are independently covalently linked to an oligonucleotide at a linkage site represented by the lysine (K) residue in a sequence a sequence motif VNHKPSN (SEQ ID NO: 58) of the heavy chain constant region of each antibody. In some embodiments, at least 15% (e.g. at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, or at least 45%) of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody. In some embodiments, 15%-45% (e.g., 15%-45%, 15%-40%, 15%-30%, 15%-20%, 20%-45% 20%-40%, 20%-30%, 30%-45% 30%-40%, or 40%-45%) of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody. For example, in some embodiments, 15%-45%, 15%-40%, 15%-35%, 15%-30%, 15%-25%, 15%-20%, 20%-45%, 20%-40%, 20%-35%, 20%-30%, 20%-25%, 25%-45%, 25%-40%, 25%-35%, 25%-30%, 30%-45%, 30%-40%, 30%-35%, 35%-45%, 35%-40%, or 40%-45% of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody. In some embodiments about 15%-30% (e.g. about 15%, about 16%, about 17%, about 18%, about 19%, about 20%, about 21%, about 22%, about 23%, about 24%, about 25%, about 26%, about 27%, about 28%, about 29%, or about 30%) of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody.

In some embodiments, the heavy chain constant regions of the antibodies of the complexes in the composition are independently covalently linked to an oligonucleotide at linkage sites represented by lysine (K) residues in a sequence motif VDKKVEP (SEQ ID NO: 61) of the heavy chain constant region of each antibody. For example, in some embodiments, the linkage site is represented by the K at position 3 of a sequence motif VDKKVEP (SEQ ID NO: 61) of the light chain constant region of each antibody. In some embodiments, the linkage site is represented by the K at position 4 of a sequence motif VDKKVEP (SEQ ID NO: 61) of the light chain constant region of each antibody. In some embodiments, the linkage sites are represented by the K at position 3 and the K at position 4 of a sequence motif VDKKVEP (SEQ ID NO: 61) of the light chain constant region of each antibody. In some embodiments, at least 5% (e.g., at least 5%, at least 6%, at least 7%, at least 8%, at least 9%, at least 10%, at least 11%, at least 12%, at least 13%, at least 14%, at least 15%) of the heavy chain constant regions of the antibodies of the complexes in the composition are independently covalently linked to an oligonucleotide at linkage sites represented by lysine (K) residues in a sequence motif VDKKVEP (SEQ ID NO: 61) of the heavy chain constant region of each antibody. In some embodiments, 5%-15% (about 5%, about 6%, about 7%, about 8%, about 9%, about 10%, about 11%, about 12%, about 13%, about 14%, or about 15%) of the heavy chain constant regions of the antibodies of the complexes in the composition are independently covalently linked to an oligonucleotide at linkage sites represented by lysine (K) residues in a sequence motif VDKKVEP (SEQ ID NO: 61) of the heavy chain constant region of each antibody.

In some embodiments, the heavy chain variable regions of the antibodies of the complexes in the composition are independently covalently linked to an oligonucleotide at a linkage site represented by the lysine (K) residue in a sequence motif GLVKPSQ (SEQ ID NO: 63) of the heavy chain variable region of each antibody. In some embodiments, at least 2% (e.g., at least 2%, at least 3%, at least 4%, at least 5%, at least 6%, at least 7%, at least 8%, at least 9%, or at least 10%) of the heavy chain variable regions of the antibodies of the complexes in the composition are independently covalently linked to an oligonucleotide at a linkage site represented by lysine (K) residue in a sequence motif GLVKPSQ (SEQ ID NO: 63) of the heavy chain variable region of each antibody. In some embodiments, 2%-10% (about 2%, about 3%, about 4%, about 5%, about 6%, about 7%, about 8%, about 9%, or about 10%) of the heavy chain variable regions of the antibodies of the complexes in the composition are independently covalently linked to an oligonucleotide at a linkage site represented by lysine (K) residue in a sequence motif GLVKPSQ (SEQ ID NO: 63) of the heavy chain variable region of each antibody.

In some embodiments, the heavy chain variable regions of the antibodies of the complexes in the composition are independently covalently linked to an oligonucleotide at a linkage site represented by the lysine (K) residue in a sequence motif PPGKGLE (SEQ ID NO: 64) of the heavy chain variable region of each antibody. In some embodiments, at least 2% (e.g., at least 2%, at least 3%, at least 4%, at least 5%, at least 6%, at least 7%, at least 8%, at least 9%, or at least 10%) of the heavy chain variable regions of the antibodies of the complexes in the composition are independently covalently linked to an oligonucleotide at a linkage site represented by the lysine (K) residue in a sequence motif PPGKGLE (SEQ ID NO: 64) of the heavy chain variable region of each antibody. In some embodiments, 2%-10% (about 2%, about 3%, about 4%, about 5%, about 6%, about 7%, about 8%, about 9%, or about 10%) of the heavy chain variable regions of the antibodies of the complexes in the composition are independently covalently linked to an oligonucleotide at a linkage site represented by the lysine (K) residue in a sequence motif PPGKGLE (SEQ ID NO: 64) of the heavy chain variable region of each antibody.

In some embodiments, the heavy chain variable regions of the antibodies of the complexes in the composition are independently covalently linked to an oligonucleotide at a linkage site represented by the lysine (K) residue in a sequence motif FSLKLSS (SEQ ID NO: 66) of the heavy chain variable region of each antibody. In some embodiments, at least 2% (e.g., at least 2%, at least 3%, at least 4%, at least 5%, at least 6%, at least 7%, at least 8%, at least 9%, or at least 10%) of the heavy chain variable regions of the antibodies of the complexes in the composition are independently covalently linked to an oligonucleotide at a linkage site represented by the lysine (K) residue in a sequence motif FSLKLSS (SEQ ID NO: 66) of the heavy chain variable region of each antibody. In some embodiments, 2%-10% (about 2%, about 3%, about 4%, about 5%, about 6%, about 7%, about 8%, about 9%, or about 10%) of the heavy chain variable regions of the antibodies of the complexes in the composition are independently covalently linked to an oligonucleotide at a linkage site represented by the lysine (K) residue in a sequence motif FSLKLSS (SEQ ID NO: 66) of the heavy chain variable region of each antibody.

In some embodiments, the light chain constant regions of the antibodies of the complexes in the composition are independently covalently linked to an oligonucleotide at a linkage site represented by the lysine (K) residue in a sequence motif EQLKSGT (SEQ ID NO: 67) of the light chain constant region of each antibody. In some embodiments, at least 2% (e.g., at least 2%, at least 3%, at least 4%, at least 5%, at least 6%, at least 7%, at least 8%, at least 9%, or at least 10%) of the light chain constant regions of the antibodies of the complexes in the composition are independently covalently linked to an oligonucleotide at a linkage site represented by the lysine (K) residue in a sequence motif EQLKSGT (SEQ ID NO: 67) of the light chain constant region of each antibody. In some embodiments, 2%-10% (about 2%, about 3%, about 4%, about 5%, about 6%, about 7%, about 8%, about 9%, or about 10%) of the light chain constant regions of the antibodies of the complexes in the composition are independently covalently linked to an oligonucleotide at a linkage site represented by the lysine (K) residue in a sequence motif EQLKSGT (SEQ ID NO: 67) of the light chain constant region of each antibody.

In some embodiments, the light chain constant regions of the antibodies of the complexes in the composition are independently covalently linked to an oligonucleotide at a linkage site represented by the lysine (K) residue in a sequence motif REAKVQW (SEQ ID NO: 68) of the light chain constant region of each antibody. In some embodiments, at least 2% (e.g., at least 2%, at least 3%, at least 4%, at least 5%, at least 6%, at least 7%, at least 8%, at least 9%, or at least 10%) of the light chain constant regions of the antibodies of the complexes in the composition are independently covalently linked to an oligonucleotide at a linkage site represented by the lysine (K) residue in a sequence motif REAKVQW (SEQ ID NO: 68) of the light chain constant region of each antibody. In some embodiments, 2%-10% (about 2%, about 3%, about 4%, about 5%, about 6%, about 7%, about 8%, about 9%, or about 10%) of the light chain constant regions of the antibodies of the complexes in the composition are independently covalently linked to an oligonucleotide at a linkage site represented by the lysine (K) residue in a sequence motif REAKVQW (SEQ ID NO: 68) of the light chain constant region of each antibody.

In some embodiments, the light chain constant regions of the antibodies of the complexes in the composition are independently covalently linked to an oligonucleotide at a linkage site represented by the lysine (K) residue in a sequence motif VQWKVDN (SEQ ID NO: 69) of the light chain constant region of each antibody. In some embodiments, at least 2% (e.g., at least 2%, at least 3%, at least 4%, at least 5%, at least 6%, at least 7%, at least 8%, at least 9%, or at least 10%) of the light chain constant regions of the antibodies of the complexes in the composition are independently covalently linked to an oligonucleotide at a linkage site represented by the lysine (K) residue in a sequence motif VQWKVDN (SEQ ID NO: 69) of the light chain constant region of each antibody. In some embodiments, 2%-10% (about 2%, about 3%, about 4%, about 5%, about 6%, about 7%, about 8%, about 9%, or about 10%) of the light chain constant regions of the antibodies of the complexes in the composition are independently covalently linked to an oligonucleotide at a linkage site represented by the lysine (K) residue in a sequence motif VQWKVDN (SEQ ID NO: 69) of the light chain constant region of each antibody.

In some embodiments, the light chain constant regions of the antibodies of the complexes in the composition are independently covalently linked to an oligonucleotide at a linkage site represented by the lysine (K) residue in a sequence motif PVTKSFN (SEQ ID NO: 70) of the light chain constant region of each antibody. In some embodiments, at least 2% (e.g., at least 2%, at least 3%, at least 4%, at least 5%, at least 6%, at least 7%, at least 8%, at least 9%, or at least 10%) of the light chain constant regions of the antibodies of the complexes in the composition are independently covalently linked to an oligonucleotide at a linkage site represented by the lysine (K) residue in a sequence motif PVTKSFN (SEQ ID NO: 70) of the light chain constant region of each antibody. In some embodiments, 2%-10% (about 2%, about 3%, about 4%, about 5%, about 6%, about 7%, about 8%, about 9%, or about 10%) of the light chain constant regions of the antibodies of the complexes in the composition are independently covalently linked to an oligonucleotide at a linkage site represented by the lysine (K) residue in a sequence motif PVTKSFN (SEQ ID NO: 70) of the light chain constant region of each antibody.

In some embodiments, the light chain variable regions of the antibodies of the complexes in the composition are independently covalently linked to an oligonucleotide at a linkage site represented by the lysine (K) residue in a sequence motif YQQKPGQ (SEQ ID NO: 71) of the light chain variable region of each antibody. In some embodiments, at least 2% (e.g., at least 2%, at least 3%, at least 4%, at least 5%, at least 6%, at least 7%, at least 8%, at least 9%, or at least 10%) of the light chain variable regions of the antibodies of the complexes in the composition are independently covalently linked to an oligonucleotide at a linkage site represented by the lysine (K) residue in a sequence motif YQQKPGQ (SEQ ID NO: 71) of the light chain variable region of each antibody. In some embodiments, 2%-10% (about 2%, about 3%, about 4%, about 5%, about 6%, about 7%, about 8%, about 9%, or about 10%) of the light chain variable regions of the antibodies of the complexes in the composition are independently covalently linked to an oligonucleotide at a linkage site represented by the lysine (K) residue in a sequence motif YQQKPGQ (SEQ ID NO: 71) of the light chain variable region of each antibody.

In some embodiments, the light chain variable regions of the antibodies of the complexes in the composition are independently covalently linked to an oligonucleotide at a linkage site represented by the lysine (K) residue in a sequence motif LEIKRTV (SEQ ID NO: 72) of the light chain variable region of each antibody. In some embodiments, at least 2% (e.g., at least 2%, at least 3%, at least 4%, at least 5%, at least 6%, at least 7%, at least 8%, at least 9%, or at least 10%) of the light chain variable regions of the antibodies of the complexes in the composition are independently covalently linked to an oligonucleotide at a linkage site represented by the lysine (K) residue in a sequence motif LEIKRTV (SEQ ID NO: 72) of the light chain variable region of each antibody. In some embodiments, 2%-10% (about 2%, about 3%, about 4%, about 5%, about 6%, about 7%, about 8%, about 9%, or about 10%) of the light chain variable regions of the antibodies of the complexes in the composition are independently covalently linked to an oligonucleotide at a linkage site represented by the lysine (K) residue in a sequence motif LEIKRTV (SEQ ID NO: 72) of the light chain variable region of each antibody.

In some embodiments, the heavy chain constant regions of the antibodies of the complexes in the composition are independently covalently linked to an oligonucleotide at a linkage site represented by the lysine (K) residue in a sequence motif ASTKGPS (SEQ ID NO: 59) of the heavy chain constant region of each antibody.

In some embodiments, the heavy chain constant regions of the antibodies of the complexes in the composition are independently covalently linked to an oligonucleotide at a linkage site represented by the lysine (K) residue in a sequence motif SNTKVD (SEQ ID NO: 60) of the heavy chain constant region of each antibody.

In some embodiments, the heavy chain constant regions of the antibodies of the complexes in the composition are independently covalently linked to an oligonucleotide at a linkage site represented by the lysine (K) residue in a sequence motif VEPKSCD (SEQ ID NO: 62) of the heavy chain constant region of each antibody.

In some embodiments, the heavy chain variable regions of the antibodies of the complexes in the composition are independently covalently linked to an oligonucleotide at a linkage site represented by the lysine (K) residue in a sequence motif PSLKNRV (SEQ ID NO: 65) of the heavy chain variable region of each antibody.

The term “about” as used herein, refers to a ±5%-10% of variation based on the % the term is used to modify.

### Antibodies

In some embodiments, complexes described herein comprise an antibody that binds human transferrin receptor 1 (TfR1). An example human transferrin receptor 1 amino acid sequence, corresponding to NCBI sequence NP_003225.2 (transferrin receptor protein 1 isoform 1, Homo sapiens) is as follows:

(SEQ ID NO: 35)
MMDQARSAFSNLFGGEPLSYTRFSLARQVDGDNSHVEMKLAVDEEENAD
NNTKANVTKPKRCSGSICYGTIAVIVFFLIGFMIGYLGYCKGVEPKTEC
ERLAGTESPVREEPGEDFPAARRLYWDDLKRKLSEKLDSTDFTGTIKLL
NENSYVPREAGSQKDENLALYVENQFREFKLSKVWRDQHFVKIQVKDSA
QNSVIIVDKNGRLVYLVENPGGYVAYSKAATVTGKLVHANFGTKKDFED
LYTPVNGSIVIVRAGKITFAEKVANAESLNAIGVLIYMDQTKFPIVNAE
LSFFGHAHLGTGDPYTPGFPSFNHTQFPPSRSSGLPNIPVQTISRAAAE
KLFGNMEGDCPSDWKTDSTCRMVTSESKNVKLTVSNVLKEIKILNIFGV
IKGFVEPDHYVVVGAQRDAWGPGAAKSGVGTALLLKLAQMFSDMVLKDG
FQPSRSIIFASWSAGDFGSVGATEWLEGYLSSLHLKAFTYINLDKAVLG
TSNFKVSASPLLYTLIEKTMQNVKHPVTGQFLYQDSNWASKVEKLTLDN
AAFPFLAYSGIPAVSFCFCEDTDYPYLGTTMDTYKELIERIPELNKVAR
AAAEVAGQFVIKLTHDVELNLDYERYNSQLLSFVRDLNQYRADIKEMGL
SLQWLYSARGDFFRATSRLTTDFGNAEKTDRFVMKKLNDRVMRVEYHFL
SPYVSPKESPFRHVFWGSGSHTLPALLENLKLRKQNNGAFNETLFRNQL
ALATWTIQGAANALSGDVWDIDNEF.

Table 2 provides examples of sequences of an anti-TfR1 antibody useful in the complexes provided herein.

TABLE 2
Examples of anti-TfR1 antibody sequences

| antibody | IMGT | Kabat | Chothia |
|---|---|---|---|
| CDR-H1 | GYSITSGYY | SGYYWN | GYSITSGY |
| | (SEQ ID NO: 1) | (SEQ ID NO: 7) | (SEQ ID NO: 12) |
| CDR-H2 | ITFDGAN | YITFDGANNYNPSL | FDG |
| | (SEQ ID NO: 2) | KN (SEQ ID NO: 8) | (SEQ ID NO: 13) |
| CDR-H3 | TRSSYDYDVL | SSYDYDVLDY | SYDYDVLD |
| | DY (SEQ ID | (SEQ ID NO: 9) | (SEQ ID NO: 14) |
| | NO: 3) | | |
| CDR-L1 | QDISNF | RASQDISNFLN | SQDISNF |
| | (SEQ ID NO: 4) | (SEQ ID NO: 10) | (SEQ ID NO: 15) |
| CDR-L2 | YTS | YTSRLHS | YTS |
| | (SEQ ID NO: 5) | (SEQ ID NO: 11) | (SEQ ID NO: 5) |
| CDR-L3 | QQGHTLPYT | QQGHTLPYT | GHTLPY |
| | (SEQ ID NO: 6) | (SEQ ID NO: 6) | (SEQ ID NO: 16) |

| | |
|---|---|
| VH | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSS (SEQ ID NO: 17) |
| VL | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTFG |
| | QGTKLEIK (SEQ ID NO: 18) |
| Fab HC | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSSASTKGPSVFPLAPSSKSTSGGTAALGC |
| | LVKDYFPEPVTVSWNSGALTSGVHTFPAVLQSSGLYSLSSVVTVPSSSL |
| | GTQTYICNVNHKPSNTKVDKKVEPKSCDKTHT (SEQ ID NO: 19) |
| Fab LC | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTFG |
| | QGTKLEIKRTVAAPSVFIFPPSDEQLKSGTASVVCLLNNFYPREAKVQW |
| | KVDNALQSGNSQESVTEQDSKDSTYSLSSTLTLSKADYEKHKVYACEV |
| | THQGLSSPVTKSFNRGEC (SEQ ID NO: 20) |

In some embodiments, the anti-TfR1 antibody of the present disclosure comprises a heavy chain complementarity determining region 1 (CDR-H1) of SEQ ID NO: 1 (according to the IMGT definition system), a heavy chain complementarity determining region 2 (CDR-H2) of SEQ ID NO: 2 (according to the IMGT definition system), a heavy chain complementarity determining region 3 (CDR-H3) of SEQ ID NO: 3 (according to the IMGT definition system), a light chain complementarity determining region 1 (CDR-L1) of SEQ ID NO: 4 (according to the IMGT definition system), a light chain complementarity determining region 2 (CDR-L2) of SEQ ID NO: 5 (according to the IMGT definition system), and a light chain complementarity determining region 3 (CDR-L3) of SEQ ID NO: 6 (according to the IMGT definition system).

In some embodiments, the anti-TfR1 antibody of the present disclosure comprises a heavy chain complementarity determining region 1 (CDR-H1) of SEQ ID NO: 7 (according to the Kabat definition system), a heavy chain complementarity determining region 2 (CDR-H2) of SEQ ID NO: 8 (according to the Kabat definition system), a heavy chain complementarity determining region 3 (CDR-H3) of SEQ ID NO: 9 (according to the Kabat definition system), a light chain complementarity determining region 1 (CDR-L1) of SEQ ID NO: 10 (according to the Kabat definition system), a light chain complementarity determining region 2 (CDR-L2) of SEQ ID NO: 11 (according to the Kabat definition system), and a light chain complementarity determining region 3 (CDR-L3) of SEQ ID NO: 6 (according to the Kabat definition system).

In some embodiments, the anti-TfR1 antibody of the present disclosure comprises a heavy chain complementarity determining region 1 (CDR-H1) of SEQ ID NO: 12 (according to the Chothia definition system), a heavy chain complementarity determining region 2 (CDR-H2) of SEQ ID NO: 13 (according to the Chothia definition system), a heavy chain complementarity determining region 3 (CDR-H3) of SEQ ID NO: 14 (according to the Chothia definition system), a light chain complementarity determining region 1 (CDR-L1) of SEQ ID NO: 15 (according to the Chothia definition system), a light chain complementarity determining region 2 (CDR-L2) of SEQ ID NO: 5 (according to the Chothia definition system), and a light chain complementarity determining region 3 (CDR-L3) of SEQ ID NO: 16 (according to the Chothia definition system).

In some embodiments, the anti-TfR1 antibody of the present disclosure comprises a heavy chain variable region (VH) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH comprising the amino acid sequence of SEQ ID NO: 17. Alternatively or in addition (e.g., in addition), the anti-TfR1 antibody of the present disclosure comprises a light chain variable region (VL) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL comprising the amino acid sequence of SEQ ID NO: 18.

In some embodiments, the anti-TfR1 antibody of the present disclosure comprises a VH comprising an amino acid sequence that is at least 75% (e.g., 75%, 80%, 85%, 90%, 95%, 98%, or 99%) identical in the framework regions to the VH comprising the amino acid sequence of SEQ ID NO: 17. Alternatively or in addition (e.g., in addition), in some embodiments, the anti-TfR1 antibody of the present disclosure comprises a VL comprising an amino acid sequence that is at least 75% (e.g., 75%, 80%, 85%, 90%, 95%, 98%, or 99%) identical in the framework regions to the VL comprising the amino acid sequence of SEQ ID NO: 18.

In some embodiments, the anti-TfR1 antibody of the present disclosure comprises a VH comprising the amino acid sequence of SEQ ID NO: 17. Alternatively or in addition (e.g., in addition), in some embodiments, the anti-TfR1 antibody of the present disclosure comprises a VL comprising the amino acid sequence of SEQ ID NO: 18.

In some embodiments, the anti-TfR1 antibody of the present disclosure comprises a heavy chain comprising an amino acid sequence least 75% (e.g., 75%, 80%, 85%, 90%, 95%, 98%, or 99%) identical to the amino acid sequence of SEQ ID NO: 19. In some embodiments, the anti-TfR1 antibody of the present disclosure is a Fab that comprises a heavy chain comprising an amino acid sequence least 75% (e.g., 75%, 80%, 85%, 90%, 95%, 98%, or 99%) identical to the amino acid sequence of SEQ ID NO: 19. Alternatively or in addition (e.g., in addition), the anti-TfR1 antibody of the present disclosure comprises a light chain comprising an amino acid sequence least 75% (e.g., 75%, 80%, 85%, 90%, 95%, 98%, or 99%) identical to the amino acid sequence of SEQ ID NO: 20. Alternatively or in addition (e.g., in addition), the anti-TfR1 antibody of the present disclosure is a Fab that comprises a light chain comprising an amino acid sequence least 75% (e.g., 75%, 80%, 85%, 90%, 95%, 98%, or 99%) identical to the amino acid sequence of SEQ ID NO: 20.

In some embodiments, the anti-TfR1 antibody of the present disclosure comprises a heavy chain comprising the amino acid sequence of SEQ ID NO: 19. In some embodiments, the anti-TfR1 antibody of the present disclosure is a Fab that comprises a heavy chain comprising an amino acid sequence of SEQ ID NO: 19. Alternatively or in addition (e.g., in addition), the anti-TfR1 antibody of the present disclosure comprises a light chain comprising the amino acid sequence of SEQ ID NO: 20. Alternatively or in addition (e.g., in addition), the anti-TfR1 antibody of the present disclosure is a Fab that comprises a light chain comprising the amino acid sequence of SEQ ID NO: 20.

Table 3 provides additional examples of sequences of an anti-TfR1 antibody useful in the complexes provided herein.

TABLE 3
Examples of anti-TfR1 antibody sequences

| antibody | IMGT | Kabat | Chothia |
|---|---|---|---|
| CDR-H1 | GYSFTSYW | SYWIG | GYSFTSY |
| | (SEQ ID NO: 37) | (SEQ ID NO: 43) | (SEQ ID NO: 48) |
| CDR-H2 | IYPGDSDT | IIYPGDSDTRYSPSFQG | GDS |
| | (SEQ ID NO: 38) | Q (SEQ ID NO: 44) | (SEQ ID NO: 49) |
| CDR-H3 | ARFPYDSSGYYSF | FPYDSSGYYSFDY | PYDSSGYYSFD |
| | DY (SEQ ID NO: | (SEQ ID NO: 45) | (SEQ ID NO: 50) |
| | 39) | | |
| CDR-L1 | QSISSY | RASQSISSYLN | SQSISSY |
| | (SEQ ID NO: 40) | (SEQ ID NO: 46) | (SEQ ID NO: 51) |
| CDR-L2 | AAS | AASSLQS | AAS |
| | (SEQ ID NO: 41) | (SEQ ID NO: 47) | (SEQ ID NO: 41) |
| CDR-L3 | QQSYSTPLT | QQSYSTPLT | SYSTPL |
| | (SEQ ID NO: 42) | (SEQ ID NO: 42) | (SEQ ID NO: 52) |

| | |
|---|---|
| VH | QVQLVQSGAEVKKPGESLKISCKGSGYSFTSYWIGWVRQMPGKGLEW |
| | MGIIYPGDSDTRYSPSFQGQVTISADKSISTAYLQWSSLKASDTAMYYC |
| | ARFPYDSSGYYSFDYWGQGTLVTVSS (SEQ ID NO: 54) |
| VL | DIQMTQSPSSLSASVGDRVTITCRASQSISSYLNWYQQKPGKAPKLLIYA |
| | ASSLQSGVPSRFSGSGSGTDFTLTISSLQPEDFATYYCQQSYSTPLTFGG |
| | GTKVEIK (SEQ ID NO: 55) |
| Fab HC | QVQLVQSGAEVKKPGESLKISCKGSGYSFTSYWIGWVRQMPGKGLEW |
| | MGIIYPGDSDTRYSPSFQGQVTISADKSISTAYLQWSSLKASDTAMYYC |
| | ARFPYDSSGYYSFDYWGQGTLVTVSSASTKGPSVFPLAPSSKSTSGGTA |
| | ALGCLVKDYFPEPVTVSWNSGALTSGVHTFPAVLQSSGLYSLSSVVTVP |
| | SSSLGTQTYICNVNHKPSNTKVDKKVEPKSCDKTHT (SEQ ID NO: 56) |
| Fab LC | DIQMTQSPSSLSASVGDRVTITCRASQSISSYLNWYQQKPGKAPKLLIYA |
| | ASSLQSGVPSRFSGSGSGTDFTLTISSLQPEDFATYYCQQSYSTPLTFGG |
| | GTKVEIKRTVAAPSVFIFPPSDEQLKSGTASVVCLLNNFYPREAKVQWK |
| | VDNALQSGNSQESVTEQDSKDSTYSLSSTLTLSKADYEKHKVYACEVT |
| | HQGLSSPVTKSFNRGEC (SEQ ID NO: 57) |

In some embodiments, the anti-TfR1 antibody of the present disclosure comprises a heavy chain complementarity determining region 1 (CDR-H1) of SEQ ID NO: 37 (according to the IMGT definition system), a heavy chain complementarity determining region 2 (CDR-H2) of SEQ ID NO: 38 (according to the IMGT definition system), a heavy chain complementarity determining region 3 (CDR-H3) of SEQ ID NO: 39 (according to the IMGT definition system), a light chain complementarity determining region 1 (CDR-L1) of SEQ ID NO: 40 (according to the IMGT definition system), a light chain complementarity determining region 2 (CDR-L2) of SEQ ID NO: 41 (according to the IMGT definition system), and a light chain complementarity determining region 3 (CDR-L3) of SEQ ID NO: 42 (according to the IMGT definition system).

In some embodiments, the anti-TfR1 antibody of the present disclosure comprises a heavy chain complementarity determining region 1 (CDR-H1) of SEQ ID NO: 43 (according to the Kabat definition system), a heavy chain complementarity determining region 2 (CDR-H2) of SEQ ID NO: 44 (according to the Kabat definition system), a heavy chain complementarity determining region 3 (CDR-H3) of SEQ ID NO: 45 (according to the Kabat definition system), a light chain complementarity determining region 1 (CDR-L1) of SEQ ID NO: 46 (according to the Kabat definition system), a light chain complementarity determining region 2 (CDR-L2) of SEQ ID NO: 47 (according to the Kabat definition system), and a light chain complementarity determining region 3 (CDR-L3) of SEQ ID NO: 42 (according to the Kabat definition system).

In some embodiments, the anti-TfR1 antibody of the present disclosure comprises a heavy chain complementarity determining region 1 (CDR-H1) of SEQ ID NO: 48 (according to the Chothia definition system), a heavy chain complementarity determining region 2 (CDR-H2) of SEQ ID NO: 49 (according to the Chothia definition system), a heavy chain complementarity determining region 3 (CDR-H3) of SEQ ID NO: 50 (according to the Chothia definition system), a light chain complementarity determining region 1 (CDR-L1) of SEQ ID NO: 51 (according to the Chothia definition system), a light chain complementarity determining region 2 (CDR-L2) of SEQ ID NO: 41 (according to the Chothia definition system), and a light chain complementarity determining region 3 (CDR-L3) of SEQ ID NO: 52 (according to the Chothia definition system).

In some embodiments, the anti-TfR1 antibody of the present disclosure comprises a heavy chain variable region (VH) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH comprising the amino acid sequence of SEQ ID NO: 54. Alternatively or in addition (e.g., in addition), the anti-TfR1 antibody of the present disclosure comprises a light chain variable region (VL) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL comprising the amino acid sequence of SEQ ID NO: 55.

In some embodiments, the anti-TfR1 antibody of the present disclosure comprises a VH comprising an amino acid sequence that is at least 75% (e.g., 75%, 80%, 85%, 90%, 95%, 98%, or 99%) identical in the framework regions to the VH comprising the amino acid sequence of SEQ ID NO: 56. Alternatively or in addition (e.g., in addition), in some embodiments, the anti-TfR1 antibody of the present disclosure comprises a VL comprising an amino acid sequence that is at least 75% (e.g., 75%, 80%, 85%, 90%, 95%, 98%, or 99%) identical in the framework regions to the VL comprising the amino acid sequence of SEQ ID NO: 57.

In some embodiments, the anti-TfR1 antibody of the present disclosure comprises a VH comprising the amino acid sequence of SEQ ID NO: 54. Alternatively or in addition (e.g., in addition), in some embodiments, the anti-TfR1 antibody of the present disclosure comprises a VL comprising the amino acid sequence of SEQ ID NO: 55.

In some embodiments, the anti-TfR1 antibody of the present disclosure comprises a heavy chain comprising an amino acid sequence least 75% (e.g., 75%, 80%, 85%, 90%, 95%, 98%, or 99%) identical to the amino acid sequence of SEQ ID NO: 56. In some embodiments, the anti-TfR1 antibody of the present disclosure is a Fab that comprises a heavy chain comprising an amino acid sequence least 75% (e.g., 75%, 80%, 85%, 90%, 95%, 98%, or 99%) identical to the amino acid sequence of SEQ ID NO: 56. Alternatively or in addition (e.g., in addition), the anti-TfR1 antibody of the present disclosure comprises a light chain comprising an amino acid sequence least 75% (e.g., 75%, 80%, 85%, 90%, 95%, 98%, or 99%) identical to the amino acid sequence of SEQ ID NO: 57. Alternatively or in addition (e.g., in addition), the anti-TfR1 antibody of the present disclosure is a Fab that comprises a light chain comprising an amino acid sequence least 75% (e.g., 75%, 80%, 85%, 90%, 95%, 98%, or 99%) identical to the amino acid sequence of SEQ ID NO: 57.

In some embodiments, the anti-TfR1 antibody of the present disclosure comprises a heavy chain comprising the amino acid sequence of SEQ ID NO: 56. In some embodiments, the anti-TfR1 antibody of the present disclosure is a Fab that comprises a heavy chain comprising an amino acid sequence of SEQ ID NO: 56. Alternatively or in addition (e.g., in addition), the anti-TfR1 antibody of the present disclosure comprises a light chain comprising the amino acid sequence of SEQ ID NO: 57. Alternatively or in addition (e.g., in addition), the anti-TfR1 antibody of the present disclosure is a Fab that comprises a light chain comprising the amino acid sequence of SEQ ID NO: 57.

In some embodiments, the anti-TfR1 antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. As such, it should be appreciated that an antibody specified as having a sequence comprising an N-terminal glutamate or glutamine residue encompasses antibodies that have undergone pyroglutamate formation resulting from a post-translational modification. In some embodiments, pyroglutamate formation occurs in a heavy chain sequence. In some embodiments, pyroglutamate formation occurs in a light chain sequence.

### Oligonucleotides

In some embodiments, an oligonucleotide of the complexes described herein is a single stranded oligonucleotide. In some embodiments, the oligonucleotide is useful for targeting DMD (e.g., for exon skipping). In some embodiments, an oligonucleotide that is useful for targeting DMD (e.g., for exon skipping) targets a DMD allele (e.g., a mutated DMD allele). In some embodiments, an oligonucleotide useful for targeting DMD (e.g., for exon skipping) targets a region of a DMD RNA (e.g., the Dp427m transcript of SEQ ID NO: 24). In some embodiments, an oligonucleotide useful for targeting DMD (e.g., for exon skipping) comprises a region of complementarity to a DMD RNA (e.g., the Dp427m transcript of SEQ ID NO: 23). In some embodiments, an oligonucleotide useful for targeting DMD (e.g., for exon skipping) comprises a region of complementarity to an exon (e.g., exons 8, 23, 43, 44, 45, 46, 50, 51, 52, 53, or 55) or an intron of a DMD RNA. In some embodiments, an oligonucleotide useful for targeting DMD (e.g., for exon skipping) targets a splicing donor site, a splicing acceptor site, a branch point, or an exonic splicing enhancer (ESE) of a DMD RNA (e.g., a DMD pre-mRNA encoded by Homo sapiens dystrophin (DMD) gene (e.g., NCBI Accession No. NG_012232.1). In some embodiments, an oligonucleotide useful for targeting DMD (e.g., for exon skipping) targets an exonic splicing enhancer (ESE) sequence in DMD (e.g., an ESE sequence of exon 23, 44, 45, 46, 50, 51, 52, 53, or 55).

Examples of DMD RNA sequences and exon sequences that may be targeted by an oligonucleotide of a complex are provided below.

Homo sapiens dystrophin (DMD), transcript variant Dp427m, mRNA (NCBI Reference Sequence: NM_004006.2) (SEQ ID NO: 23).

Homo sapiens dystrophin (DMD), transcript variant Dp427m, exon 51
(nucleotide positions 7554-7786 of NCBI Reference Sequence: NM_004006.2)
(SEQ ID NO: 24)
CTCCTACTCAGACTGTTACTCTGGTGACACAACCTGTGGTTACTAAGGAAACTGCC
ATCTCCAAACTAGAAATGCCATCTTCCTTGATGTTGGAGGTACCTGCTCTGGCAGA
TTTCAACCGGGCTTGGACAGAACTTACCGACTGGCTTTCTCTGCTTGATCAAGTTA
TAAAATCACAGAGGGTGATGGTGGGTGACCTTGAGGATATCAACGAGATGATCAT
CAAGCAGAAG
Homo sapiens dystrophin (DMD), transcript variant Dp427m, exon 8
(nucleotide positions 894-1075 of NCBI Reference Sequence: NM_004006.2)
(SEQ ID NO: 25)
ATGTTGATACCACCTATCCAGATAAGAAGTCCATCTTAATGTACATCACATCACTC
TTCCAAGTTTTGCCTCAACAAGTGAGCATTGAAGCCATCCAGGAAGTGGAAATGTT
GCCAAGGCCACCTAAAGTGACTAAAGAAGAACATTTTCAGTTACATCATCAAATG
CACTATTCTCAACAG
Homo sapiens dystrophin (DMD), transcript variant Dp427m, exon 23
(nucleotide positions 3194-3406 of NCBI Reference Sequence: NM_004006.2)
(SEQ ID NO: 26)
GCTTTACAAAGTTCTCTGCAAGAGCAACAAAGTGGCCTATACTATCTCAGCACCAC
TGTGAAAGAGATGTCGAAGAAAGCGCCCTCTGAAATTAGCCGGAAATATCAATCA
GAATTTGAAGAAATTGAGGGACGCTGGAAGAAGCTCTCCTCCCAGCTGGTTGAGC
ATTGTCAAAAGCTAGAGGAGCAAATGAATAAACTCCGAAAAATTCAG
Homo sapiens dystrophin (DMD), transcript variant Dp427m, exon 43
(nucleotide positions 6362-6534 of NCBI Reference Sequence: NM_004006.2)
(SEQ ID NO: 27)
AATATAAAAGATAGTCTACAACAAAGCTCAGGTCGGATTGACATTATTCATAGCA
AGAAGACAGCAGCATTGCAAAGTGCAACGCCTGTGGAAAGGGTGAAGCTACAGG
AAGCTCTCTCCCAGCTTGATTTCCAATGGGAAAAAGTTAACAAAATGTACAAGGA
CCGACAAGG
Homo sapiens dystrophin (DMD), transcript variant Dp427m, exon 44
(nucleotide positions 6535-6682 of NCBI Reference Sequence: NM_004006.2)
(SEQ ID NO: 28)
GCGATTTGACAGATCTGTTGAGAAATGGCGGCGTTTTCATTATGATATAAAGATAT
TTAATCAGTGGCTAACAGAAGCTGAACAGTTTCTCAGAAAGACACAAATTCCTGA
GAATTGGGAACATGCTAAATACAAATGGTATCTTAAG
Homo sapiens dystrophin (DMD), transcript variant Dp427m, exon 45
(nucleotide positions 6683-6858 of NCBI Reference Sequence: NM_004006.2)
(SEQ ID NO: 36)
GAACTCCAGGATGGCATTGGGCAGCGGCAAACTGTTGTCAGAACATTGAATGCAA
CTGGGGAAGAAATAATTCAGCAATCCTCAAAAACAGATGCCAGTATTCTACAGGA
AAAATTGGGAAGCCTGAATCTGCGGTGGCAGGAGGTCTGCAAACAGCTGTCAGAC
AGAAAAAAGAG
Homo sapiens dystrophin (DMD), transcript variant Dp427m, exon 46
(nucleotide positions 6859-7006 of NCBI Reference Sequence: NM_004006.2)
(SEQ ID NO: 29)
GCTAGAAGAACAAAAGAATATCTTGTCAGAATTTCAAAGAGATTTAAATGAATTT
GTTTTATGGTTGGAGGAAGCAGATAACATTGCTAGTATCCCACTTGAACCTGGAAA
AGAGCAGCAACTAAAAGAAAAGCTTGAGCAAGTCAAG
Homo sapiens dystrophin (DMD), transcript variant Dp427m, exon 50
(nucleotide positions 7445-7553 of NCBI Reference Sequence: NM_004006.2)
(SEQ ID NO: 30)
AGGAAGTTAGAAGATCTGAGCTCTGAGTGGAAGGCGGTAAACCGTTTACTTCAAG
AGCTGAGGGCAAAGCAGCCTGACCTAGCTCCTGGACTGACCACTATTGGAGCCT
Homo sapiens dystrophin (DMD), transcript variant Dp427m, exon 51
(nucleotide positions 7554-7786 of NCBI Reference Sequence: NM_004006.2)
(SEQ ID NO: 31)
CTCCTACTCAGACTGTTACTCTGGTGACACAACCTGTGGTTACTAAGGAAACTGCC
ATCTCCAAACTAGAAATGCCATCTTCCTTGATGTTGGAGGTACCTGCTCTGGCAGA
TTTCAACCGGGCTTGGACAGAACTTACCGACTGGCTTTCTCTGCTTGATCAAGTTA
TAAAATCACAGAGGGTGATGGTGGGTGACCTTGAGGATATCAACGAGATGATCAT
CAAGCAGAAG
Homo sapiens dystrophin (DMD), transcript variant Dp427m, exon 52
(nucleotide positions 7787-7904 of NCBI Reference Sequence: NM_004006.2)
(SEQ ID NO: 32)
GCAACAATGCAGGATTTGGAACAGAGGCGTCCCCAGTTGGAAGAACTCATTACCG
CTGCCCAAAATTTGAAAAACAAGACCAGCAATCAAGAGGCTAGAACAATCATTAC
GGATCGAA
Homo sapiens dystrophin (DMD), transcript variant Dp427m, exon 53
(nucleotide positions 7905-8116 of NCBI Reference Sequence: NM_004006.2)
(SEQ ID NO: 33)
TTGAAAGAATTCAGAATCAGTGGGATGAAGTACAAGAACACCTTCAGAACCGGAG
GCAACAGTTGAATGAAATGTTAAAGGATTCAACACAATGGCTGGAAGCTAAGGAA
GAAGCTGAGCAGGTCTTAGGACAGGCCAGAGCCAAGCTTGAGTCATGGAAGGAG
GGTCCCTATACAGTAGATGCAATCCAAAAGAAAATCACAGAAACCAAG
Homo sapiens dystrophin (DMD), transcript variant Dp427m, exon 55
(nucleotide positions 8272-8461 of NCBI Reference Sequence: NM_004006.2)
(SEQ ID NO: 34)
GGTGAGTGAGCGAGAGGCTGCTTTGGAAGAAACTCATAGATTACTGCAACAGTTC
CCCCTGGACCTGGAAAAGTTTCTTGCCTGGCTTACAGAAGCTGAAACAACTGCCA
ATGTCCTACAGGATGCTACCCGTAAGGAAAGGCTCCTAGAAGACTCCAAGGGAGT
AAAAGAGCTGATGAAACAATGGCAA

In some embodiments, an oligonucleotide useful for targeting DMD (e.g., for exon skipping) is 15-40 (e.g., 15-40, 15-35, 15-30, 15-25, 15-20, 20-40, 20-35, 20-30, 20-25, 25-40, 25-35, 25-30, 25-28, 28-30, 30-40, 30-32, 32-35, 30-35, or 35-40) nucleotides in length. In some embodiments, an oligonucleotide useful for targeting DMD (e.g., for exon skipping) is 15, 16, 17, 18, 19, 20, 21, 22, 23, 24, 25, 26, 27, 28, 29, or 30 nucleotides in length, optionally 20-35, or 30 nucleotides in length.

In some embodiments, an oligonucleotide useful for targeting DMD (e.g., for exon skipping) comprises a region of complementarity of at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides to a DMD RNA. In some embodiments, an oligonucleotide useful for targeting DMD (e.g., for exon skipping) comprises a region of complementarity of at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides to an exon of a DMD RNA.

In some embodiments, an oligonucleotide useful for targeting DMD (e.g., for exon skipping) comprises a region of complementarity of at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides to a DMD sequence as set forth in any one of SEQ ID NOs: 23-34.

In some embodiments, an oligonucleotide useful for targeting DMD (e.g., for exon skipping) comprises a region of complementarity of at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides to a target sequence as set forth in SEQ ID NO: 22 (CTAGAAATGCCATCTTCCTTGATGTTGGAG). In some embodiments, an oligonucleotide useful for targeting DMD (e.g., for exon skipping) comprises at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides of a sequence as set forth in SEQ ID NO: 21 (CTCCAACATCAAGGAAGATGGCATTTCTAG).

In some embodiments, an oligonucleotide useful for targeting DMD (e.g., for exon skipping) comprises the nucleotide sequence of SEQ ID NO: 21. In some embodiments, any one of the oligonucleotides provided herein is a PMO.

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, any one or more of the thymine bases (T's) in any one of the oligonucleotides provided herein (e.g., the oligonucleotide as set forth in SEQ ID NO: 21) may independently and optionally be uracil bases (U's), and/or any one or more of the U's in the oligonucleotides provided herein may independently and optionally be T's.

### Compositions

In some embodiments, compositions described herein comprise complexes (i.e., a plurality of complexes), each of which complex comprises an antibody (e.g., anti-TFR1 antibody) covalently linked to one or more oligonucleotides (e.g., phosphorodiamidate morpholino oligomer (PMO)) at a linkage site represented by a lysine (K) residue of the antibody, wherein the antibody comprises a heavy chain comprising a heavy chain variable region (VH) and a heavy chain constant region, and a light chain comprising a light chain variable region (VL) and a light chain constant region. In some embodiments, the antibody of such complexes comprises a CDR-H1, CDR-H2, CDR-H3, CDR-L1, CDR-L2, and CDR-L3 as set forth in Table 2 or Table 3.

In some embodiments, the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. For example, in some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. In some embodiments, the heavy chain constant regions of the antibodies of the complexes in the composition are independently covalently linked to an oligonucleotide at a linkage site represented by K213 (based on Kabat numbering) of the heavy chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody. In some embodiments, the light chain constant regions of the antibodies of the complexes in the composition are independently covalently linked to an oligonucleotide at linkage sites represented by represented by lysine (K) residues in a sequence motif DYEKHKVYA (SEQ ID NO: 53) of the light chain constant region of each antibody. For example, in some embodiments, the complexes are covalently linked to an oligonucleotide at a linkage site represented by the K at position 4 in a sequence motif DYEKHKVYA (SEQ ID NO: 53) of the light chain constant region. In some embodiments, the complexes are covalently linked to an oligonucleotide at a linkage site represented by the K at position 6 in a sequence motif DYEKHKVYA (SEQ ID NO: 53) of the light chain constant region. In some embodiments, the complexes are covalently linked to an oligonucleotide at a linkage site represented the K at position 4 and at a linkage site represented by the K at position 6 in a sequence motif DYEKHKVYA (SEQ ID NO: 53) of the light chain constant region. In some embodiments, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, the heavy chain constant regions of the antibodies of the complexes in the composition are independently covalently linked to an oligonucleotide at a linkage site represented by the lysine (K) residue in a sequence a sequence motif VNHKPSN (SEQ ID NO: 58) of the heavy chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in motif VNHKPSN (SEQ ID NO: 58) of the heavy chain constant region of each antibody. In some embodiments, the antibody is an anti-TfR1 Fab.

In some embodiments, compositions described herein comprise complexes (i.e., a plurality of complexes) wherein each complex is of the formula (I): [R¹]ₙ₁—R², in which each R¹ independently comprises a compound comprising an oligonucleotide (e.g., a PMO) and is covalently linked to R², wherein R² comprises an antibody (e.g., anti-TfR1 antibody) comprising a heavy chain comprising a heavy chain variable region (VH) and a heavy chain constant region, and a light chain comprising a light chain variable region (VL) and a light chain constant region. In some embodiments, each R¹ is covalently linked to R² via a linkage site represented by a lysine (K) residue of the antibody. In some embodiments, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody. In some embodiments, each R¹ is covalently linked to R² via a linkage site represented by a lysine (K) residue of the antibody. In some embodiments, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to R¹ at linkage sites represented by lysine (K) residues in a sequence motif DYEKHKVYA (SEQ ID NO: 53) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in motif VNHKPSN (SEQ ID NO: 58) of the heavy chain constant region of each antibody. In some embodiments, in each complex n1 is independently an integer of one or greater representing the number of instances of R¹ in each complex.

In some embodiments, the value of n1 of each complex in the composition is independently and optionally an integer from one up to the number of amino acid residues to which conjugation is desired or targeted (e.g., the number of lysine residues) in the antibody (R²). In some embodiments, the value of n1 of each complex in the composition is independently and optionally selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, the value of n1 of each complex in the composition is independently and optionally selected from 1, 2, 3, 4, 5, 6, 7, 8, 9, 10, 11, 12, 13, 14, 15, 16, 17, 18, 19, 20, 21, 22, 23, 24, 25 and 26. In some embodiments, the value of n1 of each complex in the composition is independently selected and optionally from an integer in the range of 1 to 27, 1 to 26, 1 to 10, 1 to 5, or 1 to 3. In some embodiments, the average value of n1 of complexes of the composition is in the range of 1 to 3, 1 to 5, 1 to 10, 1 to 26, or 1 to 27. In some embodiments, the average value of n1 of complexes of the composition is in the range of 1 to 10 (e.g., 1-10, 1-5, 1-4, 1-3, 3-10, 3-5, or 5-10).

In some embodiments, a composition described herein comprises unconjugated antibody (e.g., in trace amounts) and antibody conjugated to one or more oligonucleotides. In some embodiments, unconjugated antibody may be referred to as a compound of the structure of the formula (I): [R¹]ₙ₁—R², for which n1 is zero. Accordingly, in some embodiments, a composition for administration to a subject in the methods described herein comprises compounds (e.g., complexes) of the structure of the formula (I): [R¹]ₙ₁—R², for which each R¹ independently comprises a group comprising an oligonucleotide, R² comprises an antibody and n1 is independently an integer of zero or greater that reflects the number of instances of R¹ in each compound (e.g., complex). In some embodiments, the fraction of compounds of the structure of the formula (I): [R¹]ₙ₁—R², in a composition, for which n1 is zero, compared with all compounds of that structure in the composition for which n1 is one or greater, is less than 10%, less than 5%, less than 1% less than 0.5%, less than 0.1%, less than 0.05%, or less than 0.01%.

In some embodiments, compositions described herein comprise complexes that comprise a structure of the formula (I): [R¹]ₙ₁—R², wherein each R in a complex of a composition provided herein independently comprises a group of the formula (Ia):

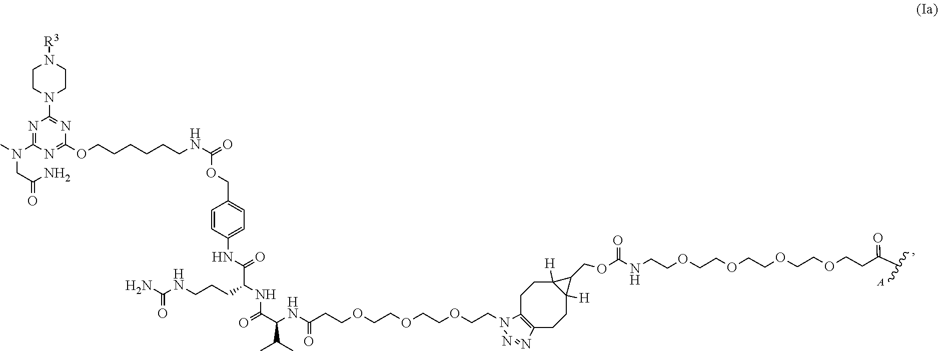

in which R³ is an oligonucleotide, e.g., a phosphorodiamidate morpholino oligomer (PMO); R² comprises an antibody comprising a heavy chain comprising a heavy chain variable region (VH) and a heavy chain constant region, and a light chain comprising a light chain variable region (VL) and a light chain constant region, wherein in each complex n1 is independently an integer (e.g., of one or greater) representing the number of instances of R¹ in each complex, and each R¹ is covalently linked to R² at attachment point A via a linkage site represented by a lysine (K) residue of the antibody. In some embodiments, the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. For example, in some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody. In some embodiments, R² comprises an anti-TfR1 antibody (e.g., a Fab) comprising a sequence as set forth in Table 2. For example, in some embodiments, R² comprises an anti-TfR1 antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody (e.g., a Fab) comprising a VH comprising the amino acid sequence of SEQ ID NO: 17 and/or comprising a VL comprising the amino acid sequence of SEQ ID NO: 18. In some embodiments, R² comprises an anti-TfR1 antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody (e.g., a Fab) comprising a heavy chain comprising the amino acid sequence of SEQ ID NO: 19 and/or comprising a light chain comprising the amino acid sequence of SEQ ID NO: 20. In some embodiments, R² comprises an antibody (e.g., a Fab) that is a Fab fragment, a full-length IgG, a Fab′ fragment, a F(ab′)2 fragment, an scFv, or an Fv. In some embodiments, R² comprises an antibody that is a Fab fragment. In some embodiments, R³ is an oligonucleotide, e.g., a phosphorodiamidate morpholino oligomer (PMO) comprising the base sequence of CTCCAACATCAAGGAAGATGGCATTTCTAG (SEQ ID NO: 21). In some embodiments, in each complex n1 is independently an integer (e.g., an integer in the range of 1-27, 1-26, 1-10, 1-5, or 1-3). In some embodiments, the average value of n1 of complexes of the composition is in the range of 1 to 10 (e.g., 1-10, 1-5, 1-4, 1-3, 3-10, 3-5, or 5-10). In some embodiments, compositions described herein comprise complexes that comprise a structure of the formula (I): [R¹]ₙ₁—R², wherein n1 is 0.

In some embodiments, compositions described herein comprise complexes that comprise a structure of the formula (I): [R¹]ₙ₁—R², in which each instance of R¹ in a complex of a composition provided herein comprises a group of the formula (Ib):

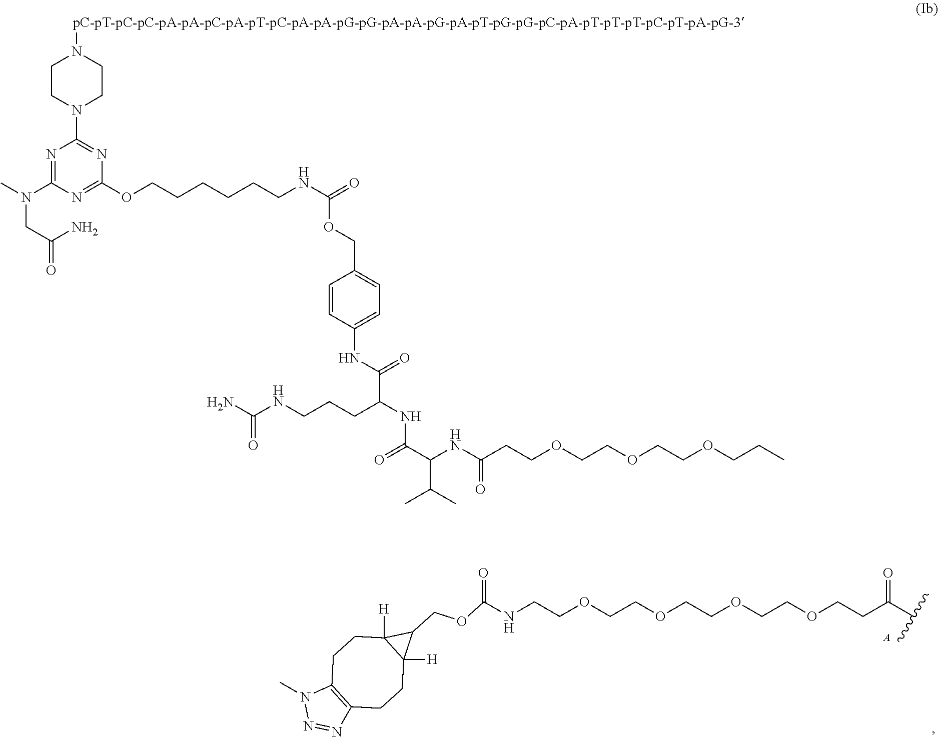

wherein -p is a phosphorodiamidate linkage of a phosphorodiamidate morpholino oligomer (PMO), and wherein the PMO comprises a base sequence of CTCCAACATCAAGGAAGATGGCATTTCTAG (SEQ ID NO: 21), R² comprises an antibody comprising a heavy chain comprising a heavy chain variable region (VH) and a heavy chain constant region, and a light chain comprising a light chain variable region (VL) and a light chain constant region, wherein in each complex n1 is independently an integer (e.g., of one or greater) representing the number of instances of R¹ in each complex, and each R¹ is covalently linked to R² at attachment point A via a linkage site represented by a lysine (K) residue of the antibody. In some embodiments, the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. For example, in some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody. In some embodiments, R² comprises an anti-TfR1 antibody (e.g., a Fab) comprising a sequence as set forth in Table 2. For example, in some embodiments, R² comprises an anti-TfR1 antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody (e.g., a Fab) comprising a VH comprising the amino acid sequence of SEQ ID NO: 17 and/or comprising a VL comprising the amino acid sequence of SEQ ID NO: 18. In some embodiments, R² comprises an anti-TfR1 antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody (e.g., a Fab) comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, the average value of n1 of complexes of the composition is in the range of 1 to 10 (e.g., 1-10, 1-5, 1-4, 1-3, 3-10, 3-5, or 5-10). In some embodiments, compositions described herein further comprise complexes that comprise a structure of the formula (I): [R¹]ₙ₁—R², wherein n1 is 0.

In some embodiments, compositions described herein comprise complexes that comprise a structure of the formula (I): [R¹]ₙ₁—R², in which each instance R¹ in a complex of a composition provided herein comprises a group of the formula (Ic):

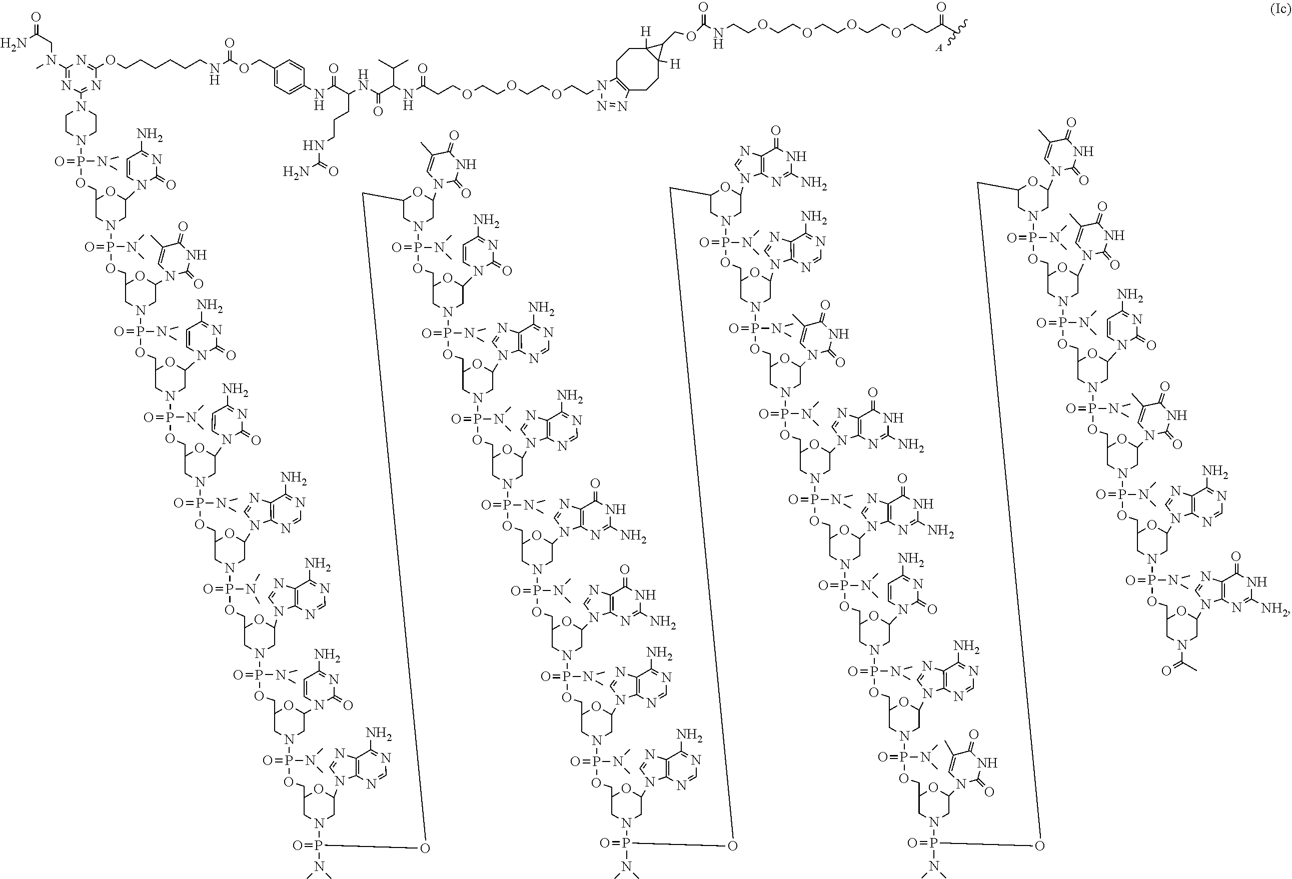

R² comprises an antibody comprising a heavy chain comprising a heavy chain variable region (VH) and a heavy chain constant region, and a light chain comprising a light chain variable region (VL) and a light chain constant region, wherein in each complex n1 is independently an integer (e.g., of one or greater) representing the number of instances of R¹ in each complex, wherein each R¹ is covalently linked to at attachment point A via a linkage site represented by a lysine (K) residue of the antibody. In some embodiments, the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. For example, in some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody. In some embodiments, R² comprises an anti-TfR1 antibody (e.g., a Fab) comprising a sequence as set forth in Table 2. For example, in some embodiments, R² comprises an anti-TfR1 antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody (e.g., a Fab) comprising a VH comprising the amino acid sequence of SEQ ID NO: 17 and/or comprising a VL comprising the amino acid sequence of SEQ ID NO: 18. In some embodiments, R² comprises an anti-TfR1 antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody (e.g., a Fab) comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, the average value of n1 of complexes of the composition is in the range of 1 to 10 (e.g., 1-10, 1-5, 1-4, 1-3, 3-10, 3-5, or 5-10). In some embodiments, compositions described herein further comprise complexes that comprise a structure of the formula (I): [R¹]ₙ₁—R², wherein n1 is 0.

In some embodiments, compositions described herein comprise complexes that comprise a structure of the formula (Id):

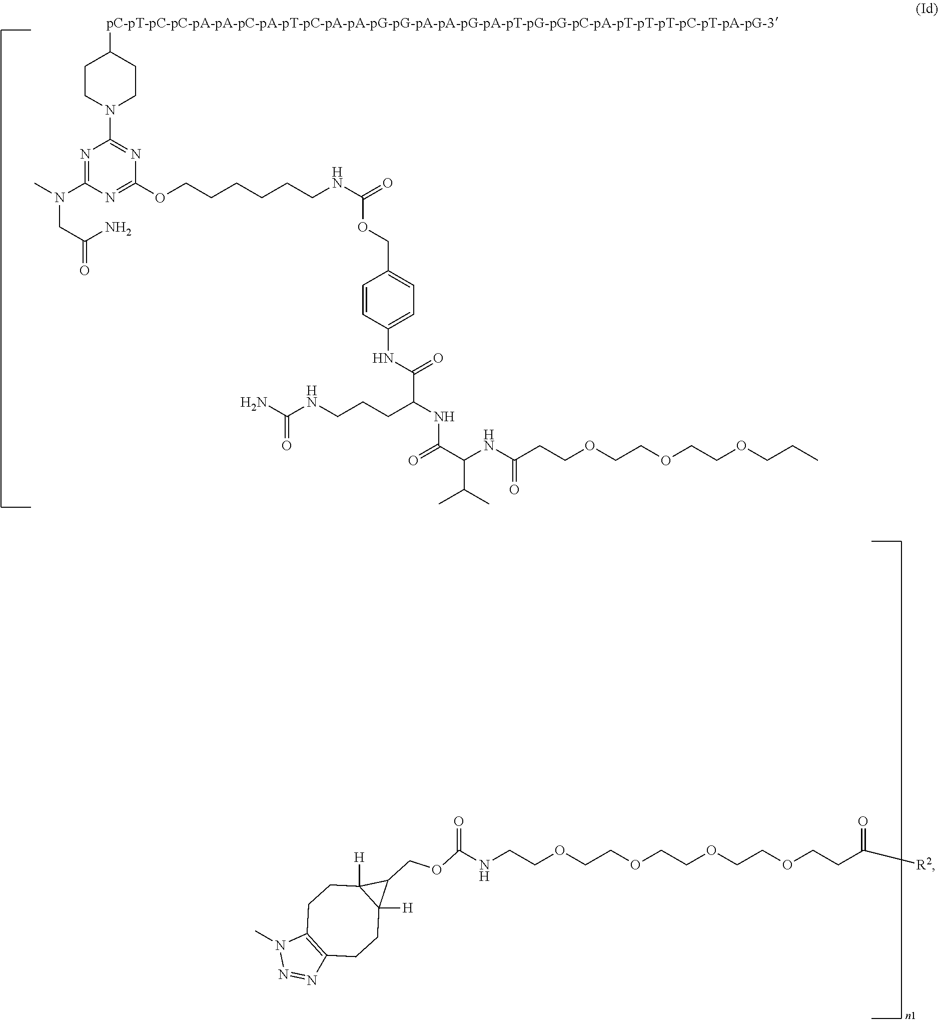

wherein -p is a phosphorodiamidate linkage of a phosphorodiamidate morpholino oligomer (PMO), and wherein the PMO comprises a base sequence of CTCCAACATCAAGGAAGATGGCATTTCTAG (SEQ ID NO: 21); wherein R² comprises an anti-TfR1 antibody (e.g., a Fab) comprising a CDR-H1, a CDR-H2, a CDR-H3, a CDR-L1, a CDR-L2, and a CDR-L3 selected from Table 2, optionally wherein the anti-TfR1 antibody (e.g., a Fab) comprises a VH comprising the amino acid sequence of SEQ ID NO: 17 and a VL comprising the amino acid sequence of SEQ ID NO: 18, further optionally wherein the anti-TfR1 antibody (e.g., a Fab) comprises a heavy chain comprising the amino acid sequence of SEQ ID NO: 19 and a light chain comprising the amino acid sequence of SEQ ID NO: 20; and wherein in each complex n1 is independently an integer (e.g., of one or greater) representing the number of instances of the group enclosed by square brackets, wherein each instance of the group enclosed by square brackets is covalently linked to a linkage site represented by a lysine (K) residue of the antibody (e.g., the Fab). In some embodiments, the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. For example, in some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of the light chain constant regions of the antibodies (e.g., the Fabs) of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody. In some embodiments, R² comprises an antibody (e.g., a Fab) comprising a sequence as set forth in Table 2. For example, in some embodiments, R² comprises an antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody (e.g., a Fab) comprising a VH comprising the amino acid sequence of SEQ ID NO: 17 and/or comprising a VL comprising the amino acid sequence of SEQ ID NO: 18. In some embodiments, R² comprises an antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody (e.g., a Fab) comprising a heavy chain comprising the amino acid sequence of SEQ ID NO: 19 and/or comprising a light chain comprising the amino acid sequence of SEQ ID NO: 20. In some embodiments, in each complex n1 is independently an integer (e.g., an integer in the range of 1-27, 1-26, 1-10, 1-5, or 1-3). In some embodiments, in each complex n1 is independently an integer of one or greater. In some embodiments, the average value of n1 of complexes of the composition is in the range of 1 to 10 (e.g., 1-10, 1-5, 1-4, 1-3, 3-10, 3-5, or 5-10). In some embodiments, compositions described herein further comprise complexes in which n1 is 0.

In some embodiments, compositions described herein comprise a structure of:

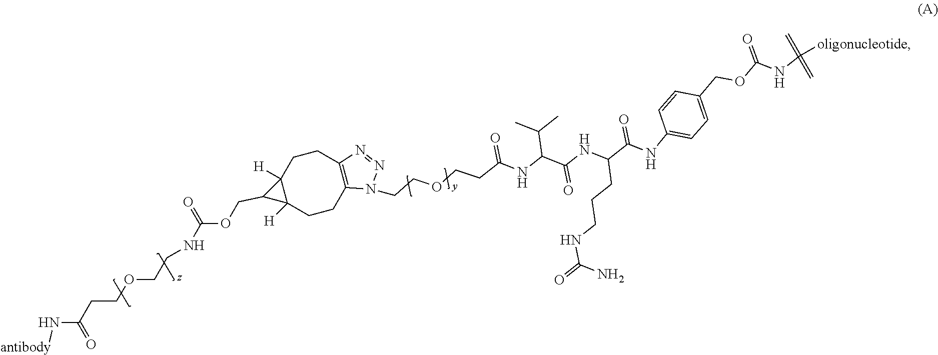

wherein y is 0-15 (e.g., 3) and z is 0-15 (e.g., 4). In some embodiments, the antibody is an an-TfR1 antibody (e.g., the anti-TfR1 antibody provided in Table 2) comprising a heavy chain comprising a heavy chain variable region (VH) and a heavy chain constant region, and a light chain comprising a light chain variable region (VL) and a light chain constant region. In some embodiments, the oligonucleotide is a PMO and comprises the base sequence of SEQ ID NO: 21. In some embodiments, the amide shown adjacent to the antibody in the structure results from a reaction with an amine of the antibody, such as a lysine epsilon amine. In some embodiments, a complex described herein comprises an anti-TfR1 Fab covalently linked to the 5′ end of a PMO via a linkage site represented by a lysine (K) residue of the antibody. In some embodiments, the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. For example, in some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to R¹ at a linkage site represented by K188 (based on Kabat numbering) and/or a linkage site represented by K190 (based on Kabat numbering) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody. In some embodiments, the anti-TfR1 antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the anti-TfR1 antibody comprises a VH comprising the amino acid sequence of SEQ ID NO: 17 and/or comprises a VL comprising the amino acid sequence of SEQ ID NO: 18. In some embodiments, the anti-TfR1 antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the anti-TfR1 antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)2 fragment, an scFv, or an Fv.

In some embodiments, compositions described herein comprise complexes that comprise a structure of the formula (I): [R¹]ₙ₁—R², wherein each R¹ in a complex of a composition provided herein independently comprises a group of the formula (Ia):

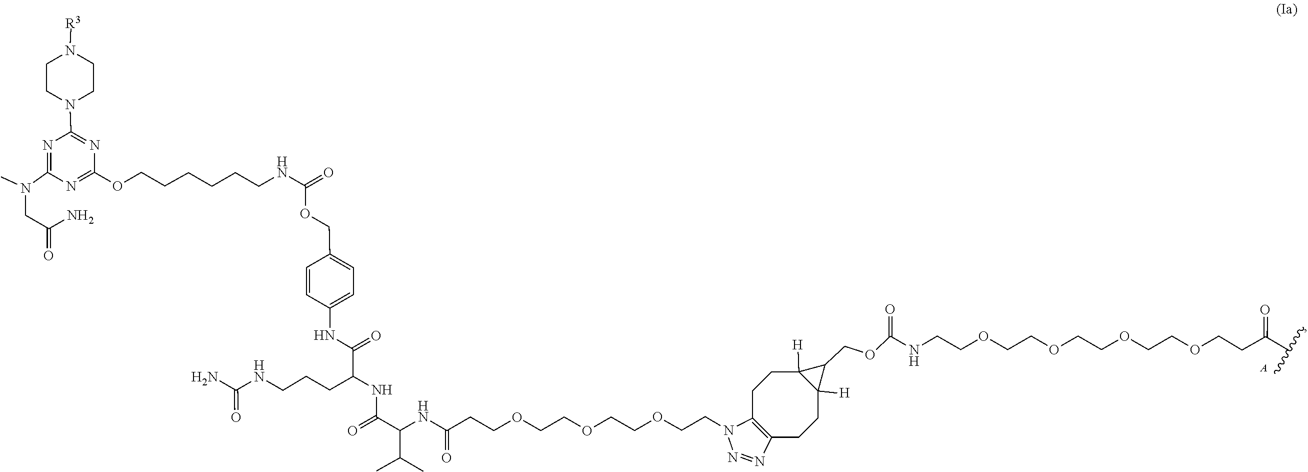

in which R³ is an oligonucleotide, e.g., a phosphorodiamidate morpholino oligomer (PMO); R² comprises an antibody comprising a heavy chain comprising a heavy chain variable region (VH) and a heavy chain constant region, and a light chain comprising a light chain variable region (VL) and a light chain constant region, wherein in each complex n1 is independently an integer (e.g., of one or greater) representing the number of instances of R¹ in each complex, and each R¹ is covalently linked to R² at attachment point A via a linkage site represented by a lysine (K) residue of the antibody. In some embodiments, the light chain constant regions of the antibodies of the complexes in the composition are independently covalently linked to an oligonucleotide at linkage sites represented by represented by lysine (K) residues in a sequence motif DYEKHKVYA (SEQ ID NO: 53) of the light chain constant region of each antibody. For example, in some embodiments, the linkage site is represented by the K at position 4 of a sequence motif DYEKHKVYA (SEQ ID NO: 53) of the light chain constant region of each antibody. In some embodiments, the linkage site is represented by the K at position 6 of a sequence motif DYEKHKVYA (SEQ ID NO: 53) of the light chain constant region of each antibody. In some embodiments, the linkage sites are represented by the K at position 4 and the K at position 6 of a sequence motif DYEKHKVYA (SEQ ID NO: 53) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody. In some embodiments, R² comprises an anti-TfR1 antibody (e.g., a Fab) comprising a sequence as set forth in Table 2. For example, in some embodiments, R² comprises an anti-TfR1 antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody comprising a VH comprising the amino acid sequence of SEQ ID NO: 17 and/or comprising a VL comprising the amino acid sequence of SEQ ID NO: 18. In some embodiments, R² comprises an anti-TfR1 antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody (e.g., a Fab) comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, R³ is an oligonucleotide, e.g., a phosphorodiamidate morpholino oligomer (PMO) comprising the base sequence of CTCCAACATCAAGGAAGATGGCATTTCTAG (SEQ ID NO: 21). In some embodiments, in each complex n1 is independently an integer (e.g., an integer in the range of 1-27, 1-26, 1-10, 1-5, or 1-3). In some embodiments, the average value of n1 of complexes of the composition is in the range of 1 to 10 (e.g., 1-10, 1-5, 1-4, 1-3, 3-10, 3-5, or 5-10). In some embodiments, compositions described herein comprise complexes that comprise a structure of the formula (I): [R¹]ₙ₁—R², wherein n1 is 0.

In some embodiments, compositions described herein comprise complexes that comprise a structure of the formula (I): [R¹]ₙ₁—R², in which each instance of R¹ in a complex of a composition provided herein comprises a group of the formula (Ib):

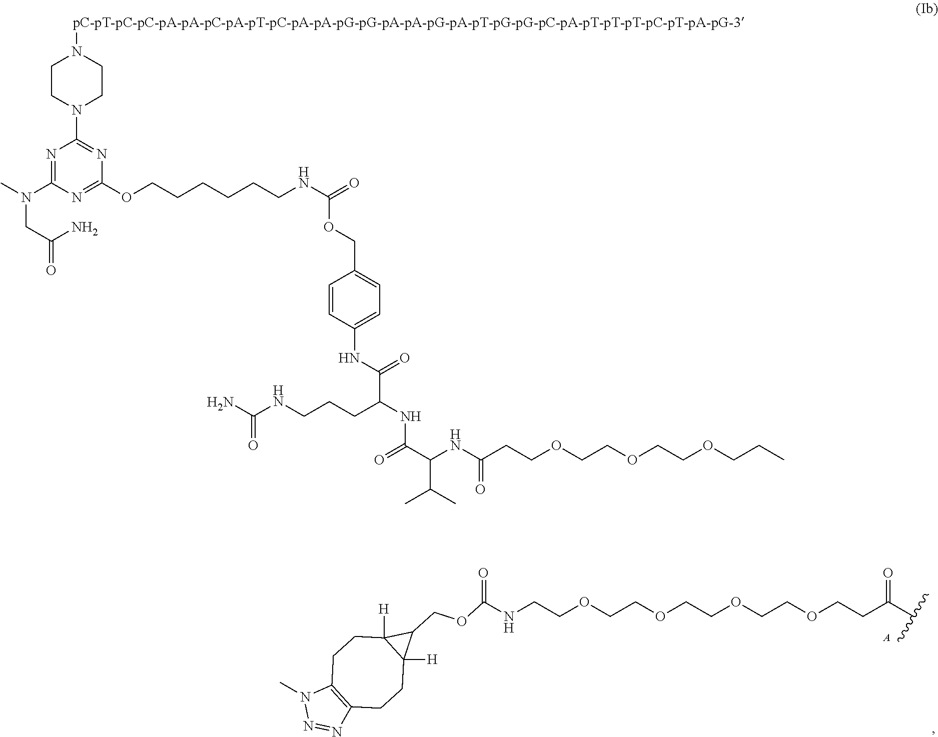

wherein -p is a phosphorodiamidate linkage of a phosphorodiamidate morpholino oligomer (PMO), and wherein the PMO comprises a base sequence of CTCCAACATCAAGGAAGATGGCATTTCTAG (SEQ ID NO: 21), R² comprises an antibody comprising a heavy chain comprising a heavy chain variable region (VH) and a heavy chain constant region, and a light chain comprising a light chain variable region (VL) and a light chain constant region, wherein in each complex n1 is independently an integer (e.g., of one or greater) representing the number of instances of R¹ in each complex, and each R¹ is covalently linked to R² at attachment point A via a linkage site represented by a lysine (K) residue of the antibody. In some embodiments, the light chain constant regions of the antibodies of the complexes in the composition are independently covalently linked to an oligonucleotide at linkage sites represented by represented by lysine (K) residues in a sequence motif DYEKHKVYA (SEQ ID NO: 53) of the light chain constant region of each antibody. For example, in some embodiments, the linkage site is represented by the K at position 4 of a sequence motif DYEKHKVYA (SEQ ID NO: 53) of the light chain constant region of each antibody. In some embodiments, the linkage site is represented by the K at position 6 of a sequence motif DYEKHKVYA (SEQ ID NO: 53) of the light chain constant region of each antibody. In some embodiments, the linkage sites are represented by the K at position 4 and the K at position 6 of a sequence motif DYEKHKVYA (SEQ ID NO: 53) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody. In some embodiments, R² comprises an anti-TfR1 antibody (e.g., a Fab) comprising a sequence as set forth in Table 2. For example, in some embodiments, R² comprises an anti-TfR1 antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody (e.g., a Fab) comprising a VH comprising the amino acid sequence of SEQ ID NO: 17 and/or comprising a VL comprising the amino acid sequence of SEQ ID NO: 18. In some embodiments, R² comprises an anti-TfR1 antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody (e.g., a Fab) comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, the average value of n1 of complexes of the composition is in the range of 1 to 10 (e.g., 1-10, 1-5, 1-4, 1-3, 3-10, 3-5, or 5-10). In some embodiments, compositions described herein further comprise complexes that comprise a structure of the formula (I): [R¹]ₙ₁—R², wherein n1 is 0.

In some embodiments, compositions described herein comprise complexes that comprise a structure of the formula (I): [R¹]ₙ₁—R², in which each instance R¹ in a complex of a composition provided herein comprises a group of the formula (Ic):

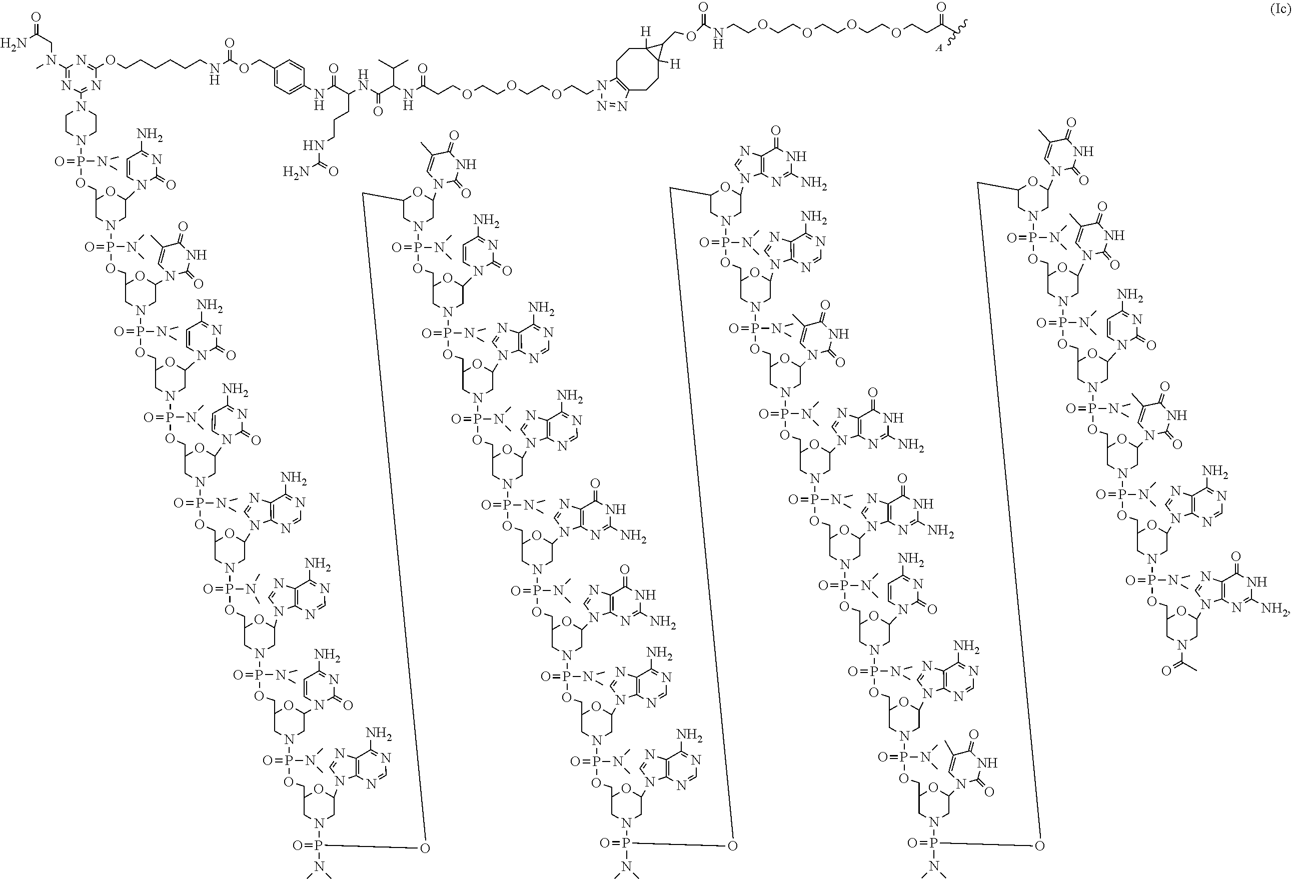

R² comprises an antibody comprising a heavy chain comprising a heavy chain variable region (VH) and a heavy chain constant region, and a light chain comprising a light chain variable region (VL) and a light chain constant region, wherein in each complex n1 is independently an integer (e.g., of one or greater) representing the number of instances of R¹ in each complex, wherein each R¹ is covalently linked to R² at attachment point A via a linkage site represented by a lysine (K) residue of the antibody. In some embodiments, the light chain constant regions of the antibodies of the complexes in the composition are independently covalently linked to an oligonucleotide at linkage sites represented by represented by lysine (K) residues in a sequence motif DYEKHKVYA (SEQ ID NO: 53) of the light chain constant region of each antibody. For example, in some embodiments, the linkage site is represented by the K at position 4 of a sequence motif DYEKHKVYA (SEQ ID NO: 53) of the light chain constant region of each antibody. In some embodiments, the linkage site is represented by the K at position 6 of a sequence motif DYEKHKVYA (SEQ ID NO: 53) of the light chain constant region of each antibody. In some embodiments, the linkage sites are represented by the K at position 4 and the K at position 6 of a sequence motif DYEKHKVYA (SEQ ID NO: 53) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody. In some embodiments, R² comprises an anti-TfR1 antibody (e.g., a Fab) comprising a sequence as set forth in Table 2. For example, in some embodiments, R² comprises an anti-TfR1 antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody (e.g., a Fab) comprising a VH comprising the amino acid sequence of SEQ ID NO: 17 and/or comprising a VL comprising the amino acid sequence of SEQ ID NO: 18. In some embodiments, R² comprises an anti-TfR1 antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody (e.g., a Fab) comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, the average value of n1 of complexes of the composition is in the range of 1 to 10 (e.g., 1-10, 1-5, 1-4, 1-3, 3-10, 3-5, or 5-10). In some embodiments, compositions described herein further comprise complexes that comprise a structure of the formula (I): [R¹]ₙ₁—R², wherein n1 is 0.

In some embodiments, compositions described herein comprise complexes that comprise a structure of the formula (Id):

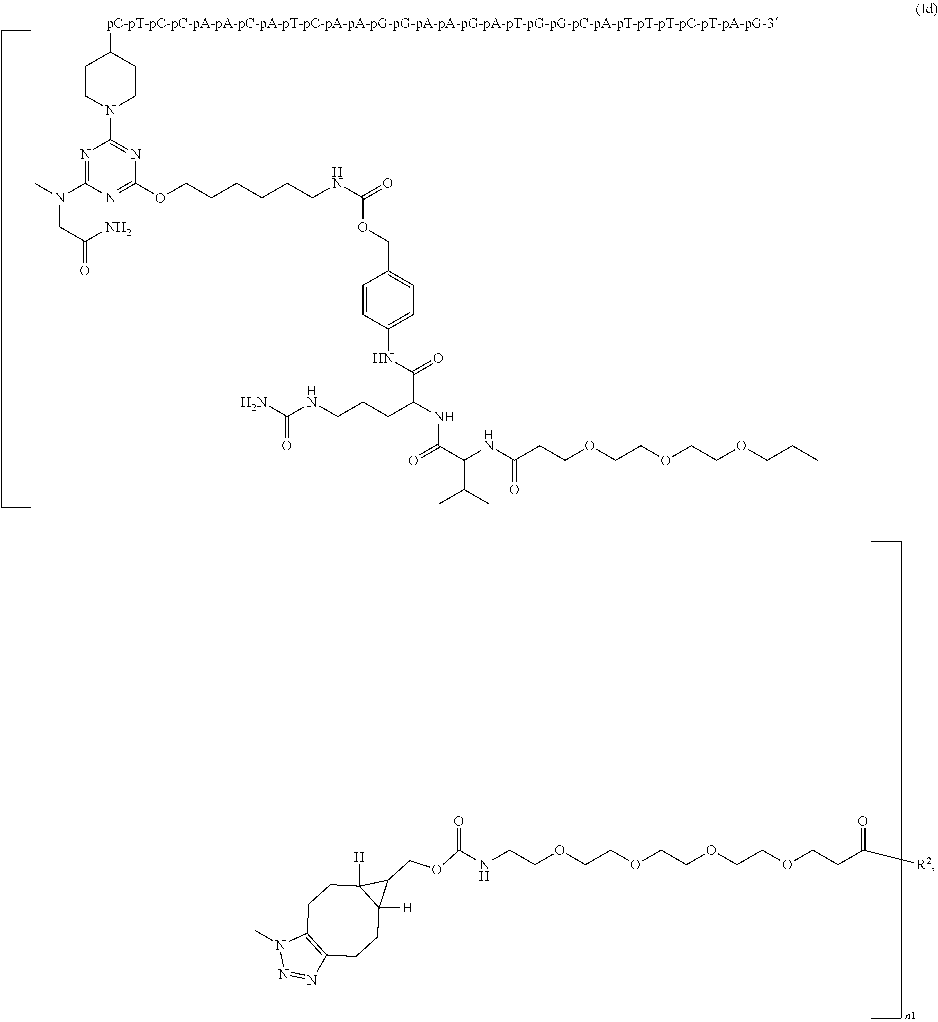

wherein -p is a phosphorodiamidate linkage of a phosphorodiamidate morpholino oligomer (PMO), and wherein the PMO comprises a base sequence of CTCCAACATCAAGGAAGATGGCATTTCTAG (SEQ ID NO: 21); wherein R² comprises an anti-TfR1 antibody (e.g., a Fab) comprising a CDR-H1, a CDR-H2, a CDR-H3, a CDR-L1, a CDR-L2, and a CDR-L3 selected from Table 2, optionally wherein the anti-TfR1 antibody (e.g., a Fab) comprises a VH comprising the amino acid sequence of SEQ ID NO: 17 and a VL comprising the amino acid sequence of SEQ ID NO: 18, further optionally wherein the anti-TfR1 antibody (e.g., a Fab) comprises a heavy chain comprising the amino acid sequence of SEQ ID NO: 19 and a light chain comprising the amino acid sequence of SEQ ID NO: 20; and wherein in each complex n1 is independently an integer (e.g., of one or greater) representing the number of instances of the group enclosed by square brackets, wherein each instance of the group enclosed by square brackets is covalently linked to a linkage site represented by a lysine (K) residue of the antibody (e.g., the Fab). In some embodiments, the light chain constant regions of the antibodies of the complexes in the composition are independently covalently linked to an oligonucleotide at linkage sites represented by represented by lysine (K) residues in a sequence motif DYEKHKVYA (SEQ ID NO: 53) of the light chain constant region of each antibody. For example, in some embodiments, the linkage site is represented by the K at position 4 of a sequence motif DYEKHKVYA (SEQ ID NO: 53) of the light chain constant region of each antibody. In some embodiments, the linkage site is represented by the K at position 6 of a sequence motif DYEKHKVYA (SEQ ID NO: 53) of the light chain constant region of each antibody. In some embodiments, the linkage sites are represented by the K at position 4 and the K at position 6 of a sequence motif DYEKHKVYA (SEQ ID NO: 53) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by a lysine (K) residue in a sequence motif DYEKHKVYA (SEQ ID NO: 53) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody. In some embodiments, R² comprises an antibody (e.g., a Fab) comprising a sequence as set forth in Table 2. For example, in some embodiments, R² comprises an antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody (e.g., a Fab) comprising a VH comprising the amino acid sequence of SEQ ID NO: 17 and/or comprising a VL comprising the amino acid sequence of SEQ ID NO: 18. In some embodiments, R² comprises an antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody (e.g., a Fab) comprising a heavy chain comprising the amino acid sequence of SEQ ID NO: 19 and/or comprising a light chain comprising the amino acid sequence of SEQ ID NO: 20. In some embodiments, in each complex n1 is independently an integer (e.g., an integer in the range of 1-27, 1-26, 1-10, 1-5, or 1-3). In some embodiments, the average value of n1 of complexes of the composition is in the range of 1 to 10 (e.g., 1-10, 1-5, 1-4, 1-3, 3-10, 3-5, or 5-10). In some embodiments, compositions described herein further comprise complexes in which n1 is 0.

In some embodiments, compositions described herein comprise a structure of:

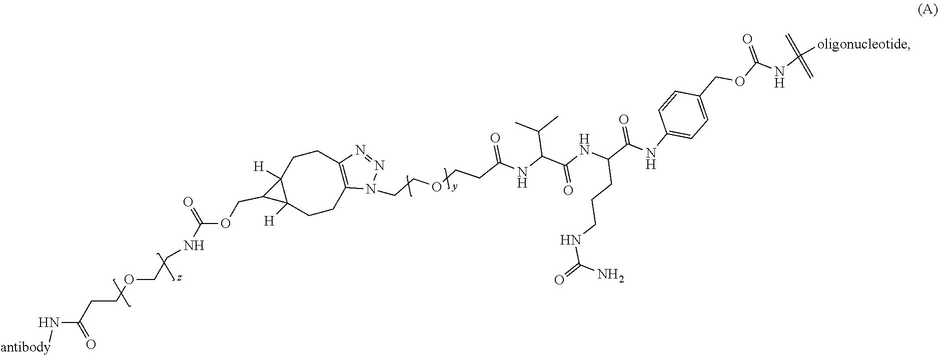

wherein y is 0-15 (e.g., 3) and z is 0-15 (e.g., 4). In some embodiments, the antibody is an an-TfR1 antibody (e.g., the anti-TfR1 antibody provided in Table 2) comprising a heavy chain comprising a heavy chain variable region (VH) and a heavy chain constant region, and a light chain comprising a light chain variable region (VL) and a light chain constant region. In some embodiments, the oligonucleotide is a PMO and comprises the base sequence of SEQ ID NO: 21. In some embodiments, the amide shown adjacent to the antibody in the structure results from a reaction with an amine of the antibody, such as a lysine epsilon amine. In some embodiments, a complex described herein comprises an anti-TfR1 Fab covalently linked to the 5′ end of a PMO via a linkage site represented by a lysine (K) residue of the antibody. In some embodiments, the light chain constant regions of the antibodies of the complexes in the composition are independently covalently linked to an oligonucleotide at linkage sites represented by represented by lysine (K) residues in a sequence motif DYEKHKVYA (SEQ ID NO: 53) of the light chain constant region of each antibody. For example, in some embodiments, the linkage site is represented by the K at position 4 of a sequence motif DYEKHKVYA (SEQ ID NO: 53) of the light chain constant region of each antibody. In some embodiments, the linkage site is represented by the K at position 6 of a sequence motif DYEKHKVYA (SEQ ID NO: 53) of the light chain constant region of each antibody. In some embodiments, the linkage sites are represented by the K at position 4 and the K at position 6 of a sequence motif DYEKHKVYA (SEQ ID NO: 53) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody. In some embodiments, the anti-TfR1 antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the anti-TfR1 antibody comprises a VH comprising the amino acid sequence of SEQ ID NO: 17 and/or comprises a VL comprising the amino acid sequence of SEQ ID NO: 18. In some embodiments, the anti-TfR1 antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the anti-TfR1 antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)2 fragment, an scFv, or an Fv.

In each composition disclosed herein, complexes (e.g., complexes comprising a structure of formula (I): [R₁]ₙ₁—R², such as complexes in which each R¹ comprises a group of the formula (Ia), (Ib), or (Ic); complexes comprising a structure of formula (Id); or complexes comprising a structure of formula (A)) of the composition may comprise a structure having the stereochemistry shown in formula (B):

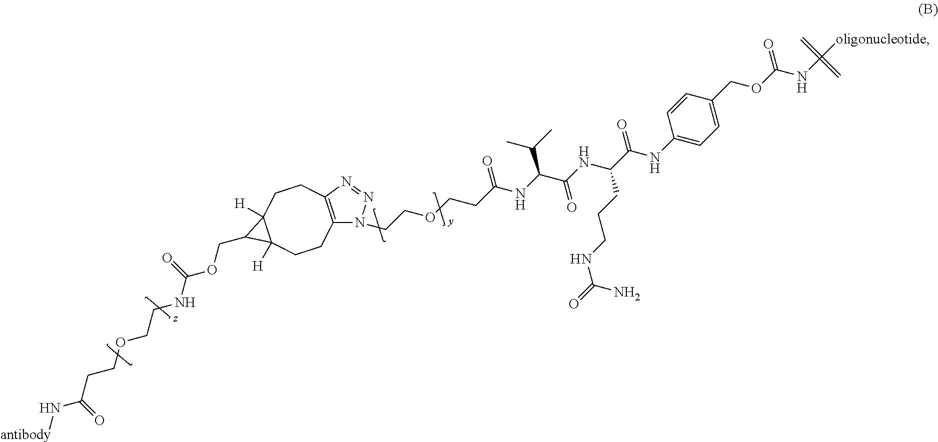

wherein y is 0-15 (e.g., 3) and z is 0-15 (e.g., 4). It should be understood that the stereochemistry shown in formula (B) can be applied to the corresponding portion of any formula or structure provided herein (e.g., formula (Ia), (Ib), (Ic), (Id), or (A)).

In some embodiments, compositions comprising complexes (i.e., a plurality of complexes) are formulated in a manner suitable for the methods described herein. In some embodiments, compositions comprising muscle-targeting complexes are delivered to a subject using a formulation that minimizes degradation, facilitates delivery and/or (e.g., and) uptake, or provides another beneficial property to the complexes in the formulation. Accordingly, in some embodiments, compositions comprising complexes (e.g., a plurality of complexes comprising a PMO covalently linked with a Fab) are formulated with histidine and/or sucrose. In some embodiments, compositions comprising muscle-targeting complexes (e.g., complexes comprising a PMO covalently linked with a Fab) are formulated with histidine and/or sucrose in aqueous solutions. In some embodiments, compositions comprising a plurality of the complexes, histidine, and sucrose can be lyophilized (e.g., for storage). In some embodiments, the lyophilized composition may be reconstituted (e.g., with water) for administration to a subject. The compositions (e.g., in aqueous solutions or in lyophilized compositions) can be suitably prepared such that when administered to a subject, either into the immediate environment of a target cell or systemically, a sufficient amount of the complexes enter target muscle cells.

In some embodiments, compositions described herein comprise complexes, wherein a concentration of the complexes in the formulation therein is between 1-50 mg/mL of the complex, optionally 10-50 mg/ml or 20-35 mg/mL (e.g., 1-10 mg/mL, 10⁻¹⁵ mg/mL, 15-20 mg/mL, 20-22 mg/mL, 22-24 mg/ml, 24-26 mg/ml, 24-25 mg/ml, 25-26 mg/ml, 22-25 mg/mL, 25-27 mg/mL, 27-29 mg/mL, 29-30 mg/mL, 25-30 mg/mL, 29-31 mg/ml, 30-31 mg/ml, 31-32 mg/ml, 30-32 mg/mL, 32-33 mg/ml, 32-35 mg/mL, 30-35 mg/mL, 35-40 mg/mL, 40-45 mg/mL, 45-50 mg/mL), optionally approximately 25 mg/mL (e.g., 25 mg/mL) or approximately 30 mg/mL (e.g., 30 mg/mL).

In some embodiments, any one or a plurality of the complexes described herein is formulated with the histidine (e.g., L-histidine) and the sucrose in an aqueous solution or in a lyophilized form (e.g., lyophilized powder).

In some embodiments, any one or a plurality of the complexes described herein is formulated with the histidine (e.g., L-histidine) and the sucrose in an aqueous solution. In some embodiments, the histidine (e.g., L-histidine) is present in the aqueous solution at a concentration in the range of 10-50 mM, 10-20 mM, 20 mM to 30 mM, or 20 mM to 40 mM, e.g., 20-22 mM, 22-24 mM, 24-25 mM, 25-26 mM, 24-26 mM, 26-27 mM, 24-27 mM, 27-28 mM, 28-29 mM, 29-30 mM, 27-30 mM, approximately 22-27 mM, approximately 23-26 mM, approximately 24-26 mM, approximately 26-28 mM, approximately 28-30 mM, approximately 30-32 mM, approximately 32-35 mM, approximately 35-40 mM, 40-45 mM, 45-50 mM, approximately 25 mM, or optionally, 25 mM. In some embodiments, the sucrose is present in the aqueous solution at a concentration in the range of 5% to 15% weight per volume (w/v %), for example, 8-15% w/v %, 9-15% w/v %, 9-11% w/v %, 9.5-11% w/v %, or for example, in the range of 5-6 w/v %, 6-7 w/v %, 7-8 w/v %, 8-9 w/v %, 9-10 w/v %, 10-11 w/v %, 11-12% w/v %, 10-12 w/v %, 12-13% w/v %, 13-14% w/v %, 12-14 w/v %, 14-15 w/v %, or 8-12 w/v %. In some embodiments, the sucrose is present in the aqueous solution at a concentration in the range of 8-12 w/v % (e.g., 10 w/v %). In some embodiments, the aqueous solution has a pH in the range of 5.0 to 7.0, for example, 5.0-5.2, 5.2-5.4, 5.4-5.6, 5.6-5.8, 5.8-6.0, 5.9-6.0, 5.9-6.1, 6.0-6.1; for example, 5.5 to 6.5, or for example, in the pH range of 5.5-5.8, 5.8-6.0, 5.9-6.1, 6.0-6.1, 6.0-6.2, 6.2-6.4, 6.4-6.5, 6.5-6.7, 6.7-6.8, 6.8-6.9, 6.9-7.0, 7.0-7.1, or 5.8-6.2. In some embodiments, the aqueous solution has a pH in the range of 5.8-6.2 (e.g., 5.8-6.0, 5.8-6.1, 5.9-6.1). In some embodiments, the aqueous solution has a pH in the range of 5.9-6.2. In some embodiments, the aqueous solution has a pH in the range of 6.0-6.1 (e.g., about 6.0, or 6.0).

In some embodiments, any one of the compositions described herein comprises one or a plurality of complexes, histidine, and sucrose, wherein the histidine (e.g., L-histidine) is present in the compositions (e.g., aqueous solution) at a concentration of 25 mM, wherein the sucrose is present in the compositions (e.g., aqueous solution) at a concentration of 10 w/v %, and wherein the compositions (e.g., aqueous solution) is at a pH of about 6.0 (e.g., 6.0, 5.9-6.1).

In some embodiments, any one of the compositions described herein comprises one or a plurality of complexes, histidine, and sucrose, wherein the histidine (e.g., L-histidine) is present in the aqueous solution at a concentration of 25 mM, wherein the sucrose is present in the aqueous solution at a concentration of 10 w/v %, and wherein the pH of about 6.0 (e.g., 6.0, 5.9-6.1), and the concentration of complexes in the formulation is 10-50 mg/ml or 20-35 mg/mL (e.g., 1-10 mg/mL, 10-15 mg/mL, 15-20 mg/mL, 20-22 mg/mL, 22-24 mg/ml, 24-26 mg/ml, 22-25 mg/mL, 25-27 mg/mL, 27-29 mg/mL, 29-31 mg/ml, 29-30 mg/mL, 30-31 mg/ml, 31-32 mg/ml, 25-30 mg/mL, 30-32 mg/mL, 32-35 mg/mL, 30-35 mg/mL, 35-40 mg/mL, 40-45 mg/mL, 45-50 mg/mL), optionally 25 mg/mL or 30 mg/mL.

As described herein, in some embodiments, compositions described herein are formulated in aqueous solutions that comprise sucrose. In some embodiments, the sucrose serves at least in part as a lyoprotectant. In some embodiments, the sucrose is from a plant, e.g., grass, fruit, or vegetable (e.g., root vegetable) source (e.g., beet (e.g., sugar beet, for example, Saccharum spp.)), sugarcane (e.g., Beta vulgaris), dates, sugar maple, sweet sorghum, apples, oranges, carrots, molasses, maple syrup, corn sweeteners) or animal product (e.g., honey). In some embodiments, the sucrose is from beet or sugarcane (e.g., beet sucrose, sugarcane sucrose). In some embodiments, a lyoprotectant other than sucrose may be used, e.g., trehalose, mannitol, lactose, polyethylene glycol, or polyvinyl pyrrolidone. However, in some embodiments, a collapse temperature modifier (e.g., dextran, ficoll, or gelatin) may be provided in a composition.

In some embodiments, provided is a product (e.g., lyophilized composition described herein), produced by a process comprising lyophilizing an aqueous solution of a composition (e.g., in aqueous form) described herein.

In some embodiments, a composition is formulated to be compatible with its intended route of administration. Examples of routes of administration include parenteral, e.g., intravenous, intradermal, subcutaneous, administration. Typically, the route of administration is intravenous or subcutaneous.

### Methods of Use/Treatment

Complexes comprising an anti-TfR1 antibody (e.g., Fab) covalently linked to a molecular payload (e.g., oligonucleotide, e.g., phosphorodiamidate morpholino oligomer (PMO)) as described herein are effective in treating a subject having a dystrophinopathy, e.g., Duchenne Muscular Dystrophy. In some embodiments, complexes comprise a molecular payload that is an oligonucleotide, e.g., an oligonucleotide that facilitates exon skipping of an mRNA expressed from a mutated DMD allele.

In some embodiments, a subject may be a human subject, a non-human primate subject, a rodent subject, or any suitable mammalian subject. In some embodiments, a subject may have Duchenne muscular dystrophy or other dystrophinopathy. In some embodiments, a subject has a mutated DMD allele, which may optionally comprise at least one mutation in a DMD exon that causes a frameshift mutation and leads to improper RNA splicing/processing. In some embodiments, a subject is suffering from symptoms of a severe dystrophinopathy, e.g. muscle atrophy or muscle loss. In some embodiments, a subject has an asymptomatic increase in serum concentration of creatine phosphokinase (CK) and/or (e.g., and) muscle cramps with myoglobinuria. In some embodiments, a subject has a progressive muscle disease, such as Duchenne or Becker muscular dystrophy or DMD-associated dilated cardiomyopathy (DCM). In some embodiments, a subject is not suffering from symptoms of a dystrophinopathy.

In some embodiments, a subject has a mutation in a DMD gene that is amenable to exon 51 skipping. In some embodiments, a complex as described herein is effective in treating a subject having a mutation in a DMD gene that is amenable to exon 51 skipping. In some embodiments, a complex comprises an oligonucleotide, e.g., an antisense oligonucleotide that facilitates skipping of exon 51 of a pre-mRNA, such as in a pre-mRNA encoded from a mutated DMD gene (e.g., a mutated DMD gene that is amenable to exon 51 skipping).

An aspect of the disclosure includes a method involving administering to a subject an effective amount of a complex as described herein. In some embodiments, an effective amount of a pharmaceutical composition that comprises a complex comprising a muscle-targeting agent covalently linked to a molecular payload can be administered to a subject in need of treatment. In some embodiments, the effective amount provides to the subject 5 mg to 100 mg (e.g., 5 mg to 100 mg, 5 mg to 80 mg, 5 mg to 50 mg, 5 mg to 40 mg, 5 mg to 30 mg, 5 mg to 20 mg, 10 mg to 60 mg, 10 mg to 50 mg, 10 mg to 40 mg, 10 mg to 30 mg, or 30 mg to 60 mg) of the oligonucleotides of the complexes per kg of the subject.

In some embodiments, a pharmaceutical composition comprising a complex as described herein may be administered by a suitable route, which may include intravenous administration, e.g., as a bolus or by continuous infusion over a period of time. In some embodiments, intravenous administration may be performed by intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

Compositions for intravenous administration may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, a pharmaceutical composition that comprises a complex comprising a muscle-targeting agent covalently linked to a molecular payload is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a muscle-targeting agent covalently linked to a molecular payload is administered at an effective concentration that confers therapeutic effect on a subject. Effective amounts vary, as recognized by those skilled in the art, depending on the severity of the disease, unique characteristics of the subject being treated, e.g. age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g. the half-life of the complex in a subject, generally will contribute to determination of the concentration of pharmaceutical composition that is used for treatment. The frequency of administration may be empirically determined and adjusted to maximize the efficacy of the treatment.

The efficacy of treatment may be assessed using any suitable methods. In some embodiments, the efficacy of treatment may be assessed by evaluation of observation of symptoms associated with a dystrophinopathy, e.g. muscle atrophy or muscle weakness, through measures of a subject's self-reported outcomes, e.g. mobility, self-care, usual activities, pain/discomfort, and anxiety/depression, or by quality-of-life indicators, e.g. lifespan.

In some embodiments, a pharmaceutical composition that comprises a complex comprising a muscle-targeting agent covalently linked to a molecular payload described herein is administered to a subject at an effective concentration sufficient to modulate activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

### Analytical Methods

In some aspects, provided herein are methods of determining a drug to antibody ratio (DAR) range of a first plurality of complexes, each complex comprising an antibody covalently linked to one or more oligonucleotides via a linker, wherein each linker comprises one or more protease cleavage sites, the method comprising: (i) removing the one or more oligonucleotides from the antibodies to which they are covalently linked of the first plurality of complexes by cleaving at least one of the one or more protease cleavage sites of the linker, wherein cleaving at least one of the one or more protease cleavage sites of the linker results in a second plurality of complexes, each complex comprising an antibody covalently linked to one or more partial linkers; (ii) obtaining the second plurality of complexes resulting from step (i); (iii) determining the masses of the complexes obtained in step (ii) via mass spectrometry; and(iv) determining the DAR range of the complexes obtained in step (ii); wherein a detected mass by mass spectrometry corresponding to the mass of the antibody plus the mass of n1 partial linkers indicates a DAR of n1, wherein n1 is an integer of one or greater. In some aspects, provided herein are methods of analyzing a first plurality of complexes, each complex comprising an antibody covalently linked to one or more oligonucleotides via a linker, wherein each linker comprises one or more protease cleavage sites, the method comprising: (i) removing the one or more oligonucleotides from the antibodies to which they are covalently linked of the first plurality of complexes by cleaving at least one of the one or more protease cleavage sites in the linker, wherein cleaving at least one of the one or more protease cleavage sites in the linker results in a second plurality of complexes, each complex comprising an antibody covalently linked to one or more partial linkers, and wherein the antibody remains intact; (ii) obtaining the second plurality complexes resulting from step (i); (iii) digesting the antibodies of complexes obtained in (ii) with a protease to obtain fragments of the antibodies; and (iv) determining the mass of the fragments of the antibodies obtained in step (iii) via mass spectrometry to identify the fragments covalently linked one or more partial linkers.

In some embodiments, each complex of the first plurality of complexes used in a method described herein, comprises an antibody covalently linked to one or more oligonucleotides, each oligonucleotide being covalently linked at a linkage site represented by a lysine (K) residue of the antibody, wherein the antibody comprises a heavy chain comprising a heavy chain variable region (VH) and a heavy chain constant region, and a light chain comprising a light chain variable region (VL) and a light chain constant region. In some embodiments, the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. For example, in some embodiments, the linkage site is represented by K188 (based on Kabat numbering) of the light chain constant region of each antibody. In some embodiments, the linkage site is represented by K190 (based on Kabat numbering) of the light chain constant region of each antibody. In some embodiments, the linkage sites are represented by K188 (based on Kabat numbering) and K190 (based on Kabat numbering) of the light chain constant region of each antibody. In some embodiments, at least 80% (e.g., 80%-98%, 80%-95%, 80%-90%, 85%-98%, 85%-95%, 85%-90%, 90%-98%, or 90%-95%) of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody. In some embodiments, the heavy chain constant regions of the antibodies of the complexes in the composition are independently covalently linked to an oligonucleotide at a linkage site represented by K213 (based on Kabat numbering) of the heavy chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by K213 (based on Kabat numbering) of the heavy chain constant region of each antibody. In some embodiments, the light chain constant regions of the antibodies of the complexes in the composition are independently covalently linked to an oligonucleotide at linkage sites represented by represented by lysine (K) residues in a sequence motif DYEKHKVYA (SEQ ID NO: 53) of the light chain constant region of each antibody. For example, in some embodiments, the complexes are covalently linked to an oligonucleotide at a linkage site represented by the K at position 4 in a sequence motif DYEKHKVYA (SEQ ID NO: 53) of the light chain constant region. In some embodiments, the complexes are covalently linked to an oligonucleotide at a linkage site represented by the K at position 6 in a sequence motif DYEKHKVYA (SEQ ID NO: 53) of the light chain constant region. In some embodiments, the complexes are covalently linked to an oligonucleotide at a linkage site represented the K at position 4 and at a linkage site represented by the K at position 6 in a sequence motif DYEKHKVYA (SEQ ID NO: 53) of the light chain constant region. In some embodiments, at least 80% (e.g., 80%-98%, 80%-95%, 80%-90%, 85%-98%, 85%-95%, 85%-90%, 90%-98%, or 90%-95%) of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody. In some embodiments, the heavy chain constant regions of the antibodies of the complexes in the composition are independently covalently linked to an oligonucleotide at a linkage site represented by the lysine (K) residue in a sequence a sequence motif VNHKPSN (SEQ ID NO: 58) of the heavy chain constant region of each antibody. In some embodiments, at least 15% (e.g., 15%-40%, 15%-30%, 15%-20%, 20%-40%, 20%-30%, or 30%-40%) of the heavy chain constant regions of the antibodies of the complexes in the composition are independently covalently linked to an oligonucleotide at a linkage site represented by the lysine (K) residue in motif VNHKPSN (SEQ ID NO: 58) of the heavy chain constant region of each antibody.

In some embodiments, in each complex of the first plurality of complexes used in a method described herein, the antibody is an anti-transferrin receptor (TfR1) antibody comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16.

In some embodiments, in each complex of the first plurality of complexes used in a method described herein, the antibody is an anti-transferrin receptor (TfR1) antibody comprising: a heavy chain complementarity determining region 1 (CDR-H1) comprising a sequence as set forth in SEQ ID NOs: 37, 43, or 48, a heavy chain complementarity determining region 2 (CDR-H2) comprising a sequence as set forth in SEQ ID NOs: 38, 44, or 49, a heavy chain complementarity determining region 3 (CDR-H3) comprising a sequence as set forth in SEQ ID NOs: 39, 45, or 50, a light chain complementarity determining region 1 (CDR-L1) comprising a sequence as set forth in SEQ ID NOs: 40, 46, 51, a light chain complementarity determining region 2 (CDR-L2) comprising a sequence as set forth in SEQ ID NOs: 41 or 47, and a light chain complementarity determining region 3 (CDR-L3) comprising a sequence as set forth in SEQ ID NOs: 42 or 52.

In some embodiments, in each complex of the first plurality of complexes used in a method described herein, the anti-TfR1 antibody is a Fab fragment, a full-length IgG, a Fab′ fragment, or a F(ab′)2 fragment. In some embodiments, the antibody is a Fab fragment. In some embodiments, in the methods described herein, the oligonucleotide is a phosphorodiamidate morpholino oligomer (PMO). In some embodiments, in each complex of the first plurality of complexes used in the methods described herein, the VH comprises an amino acid sequence at least 85% identical to SEQ ID NO: 17; and/or wherein the VL comprises an amino acid sequence at least 85% identical to SEQ ID NO: 18. In some embodiments, the antibody comprises a heavy chain variable region (VH) comprising the amino acid sequence of SEQ ID NO: 17 and a light chain variable region (VL) comprising the amino acid sequence of SEQ ID NO: 18. In some embodiments, in each complex of the first plurality of complexes used in the methods described herein, the heavy comprises an amino acid sequence at least 85% identical to SEQ ID NO: 19; and/or wherein the light chain comprises an amino acid sequence at least 85% identical to SEQ ID NO: 20. In some embodiments, the antibody comprises a heavy chain comprising the amino acid sequence of SEQ ID NO: 19 and a light chain comprising the amino acid sequence of SEQ ID NO: 20.

In some embodiments, in the methods provided herein, the cleaving of step (i) is carried out with papain. In some embodiments, the digesting of step (iii) is carried out with a chymotrypsin.

### EXAMPLES

### Example 1. Exon-Skipping Activity of Anti-TfR1 Conjugate in DMD Patient Myotubes

This study evaluated the exon-skipping activities of conjugates comprising the anti-TfR1 Fab having the heavy chain and light chain sequences shown in Table 2 covalently linked (through lysine conjugation) via a linker comprising a Valine-Citrulline sequence to a DMD exon 51-skipping oligonucleotide (oligo). The DMD exon 51-skipping oligonucleotide is a PMO and comprises the base sequence of SEQ ID NO: 21. The conjugates comprise a structure of:

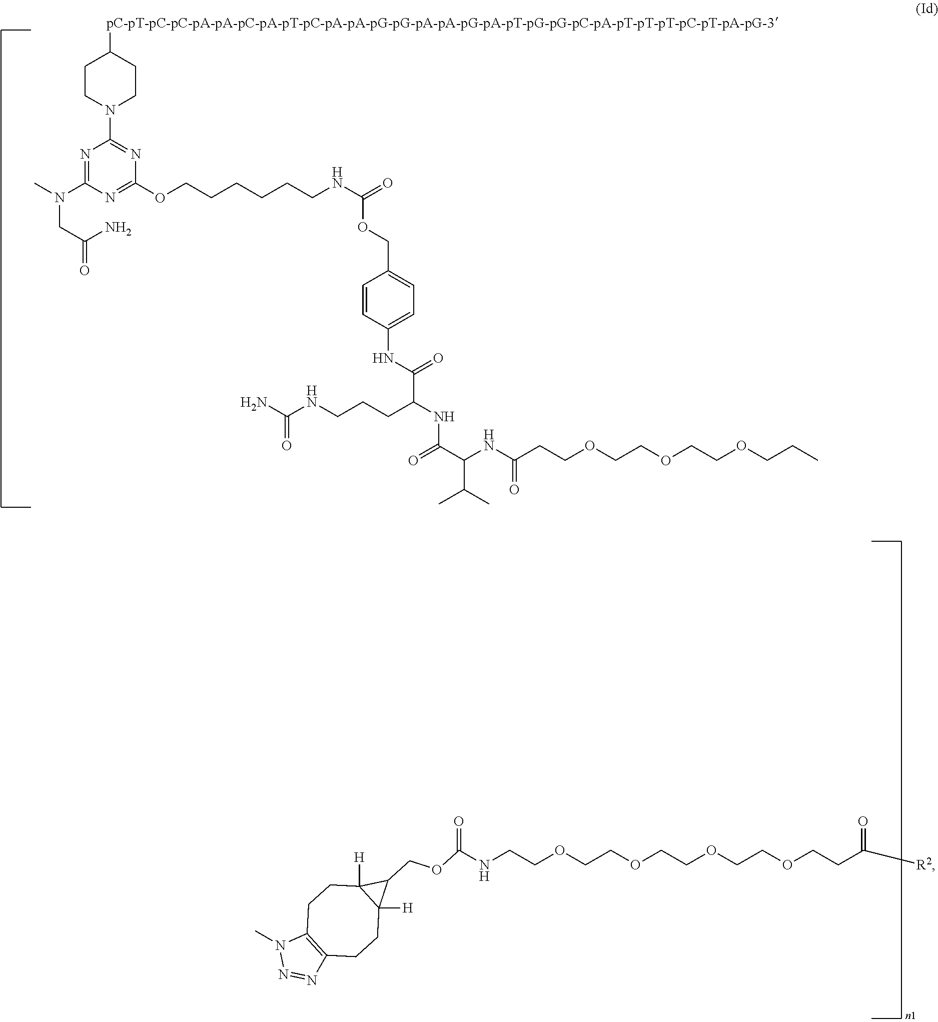

wherein R² is the anti-TfR1 Fab shown in Table 2, and wherein in each conjugate n1 is independently an integer of 1-5.

Immortalized human myoblasts bearing an exon 52 deletion were thawed and seeded at a density of 1×10⁶ cell/flask in Promocell Skeletal Cell Growth Media (with 5% FBS and 1×Pen-Strep) and allowed to grow to confluency. Once confluent, cells were trypsinized and pelleted via centrifugation and resuspended in fresh Promocell Skeletal Cell Growth Media. The cell number was counted and cells were seeded into Matrigel-coated 96-well plates at a density of 50k cells/well. Cells were allowed to recover for 24 hours. Cells were induced to differentiate by aspirating the growth media and replacing with differentiation media with no serum. Cells were then treated with the DMD exon 51-skipping oligonucleotide (not covalently linked to an antibody—“naked”) at a concentration of 10 μM oligonucleotide or treated with the conjugate to a final concentration of 10 μM oligonucleotide equivalent. Cells were incubated with test articles for ten days then total RNA was harvested from the 96 well plates. cDNA synthesis was performed on 75 ng of total RNA, and mutation specific PCRs were performed to evaluate the degree of exon 51 skipping in each cell type. Mutation-specific PCR products were run on a 4% agarose gel and visualized using SYBR gold. Densitometry was used to calculate the relative amounts of the skipped and unskipped amplicon and exon skipping was determined as a ratio of the Exon 51 skipped amplicon divided by the total amount of amplicon present:

$$\% Exon Skipping = \frac{Skipped Amplicon}{( Skipped Amplicon + Unskipped Amplicon )} * 100$$

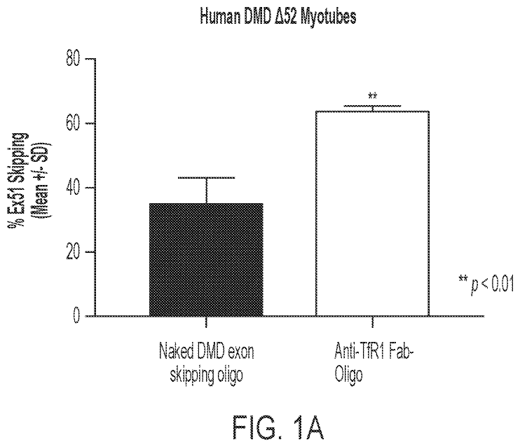

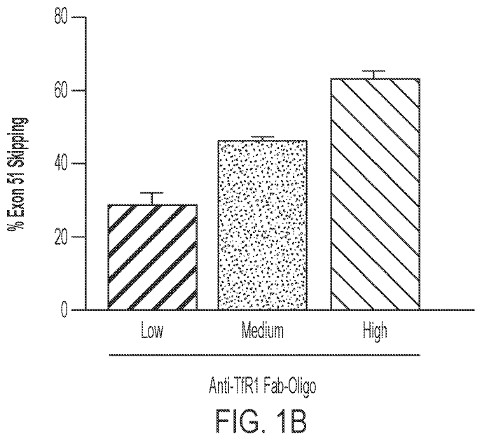

The results demonstrate that the conjugates resulted in enhanced exon skipping compared to the same DMD exon 51-skipping oligonucleotide that is not covalently linked to an antibody in patient myotubes (FIG. 1A). This indicates that an anti-TfR1 Fab (e.g., having a sequence set forth in Table 2) facilitated cellular internalization of the conjugate into muscle cells resulting in activity of the exon 51-skipping oligonucleotide in the muscle cells.

Additionally, cells were treated with anti-TfR1 Fab-oligo conjugates to a final concentration of 2.5 μM (low), 5 μM (medium), or 10 μM (high) oligonucleotide equivalent. Exon 51-skipping data is shown in FIG. 1B. The results demonstrate that the conjugates resulted in dose dependent exon 51 skipping in DMD patient myotubes.

### Example 2. Exon Skipping Activity of Anti-TfR1 Fab-Oligonucleotide Conjugate In Vivo in Cynomolgus Monkeys

The anti-TfR1-oligo conjugates described in Example 1 were tested for their exon skipping activity in vivo in healthy non-human primates. Naïve male cynomolgus monkeys (n=4-5 per group) were administered two doses of vehicle, 30 mg/kg naked oligo (i.e., not covalently linked to an antibody), or 122 mg/kg anti-TfR1 Fab covalently linked to the DMD exon 51-skipping oligonucleotide (30 mg/kg oligo equivalent) via intravenous infusion on days 1 and 8. Animals were sacrificed and tissues harvested either 2 weeks or 4 weeks after the first dose was administered. Total RNA was collected from tissue samples using a Promega Maxwell® RSC instrument and cDNA synthesis was performed using qScript cDNA SuperMix. Assessment of exon 51 skipping was performed using end-point PCR.

Capillary electrophoresis of the PCR products was used to assess exon skipping, and % exon 51 skipping was calculated using the following formula:

$$\% Exon Skipping = \frac{Molarity of Skipped Band}{Molarity of Skipped Band + Molarity of Unskipped Band} * 100.$$

Calculated exon 51 skipping results are shown in Table 4.

TABLE 4
Exon 51 skipping of dystrophin in cynomolgus monkey dystrophin

| | Time | | | | |
|---|---|---|---|---|---|
| | 2 weeks | | | 4 weeks | |
| | | Oligo | Conju- | Oligo | Conju- |
| Group | Vehicle | alone^{a} | gate | alone^{a} | gate |
| Conjugate | 0 | n/a | 122 | n/a | 122 |
| dose^{b} | | | | | |
| Oligo alone | 0 | 30 | 30 | 30 | 30 |
| Dose^{c} | | | | | |
| Quadriceps ^{d} | 0.00 | 1.216 | 4.906 | 0.840 | 1.708 |
| | (0.00) | (1.083) | (3.131) | (1.169) | (1.395) |
| Diaphragm ^{d} | 0.00 | 1.891 | 7.315 | 0.717 | 9.225 |
| | (0.00) | (2.911) | (1.532) | (1.315) | (4.696) |
| Heart ^{d} | 0.00 | 0.043 | 3.42 | 0.00 | 4.525 |
| | (0.00) | (0.096) | (1.192) | (0.00) | (1.400) |
| Biceps ^{d} | 0.00 | 0.607 | 3.129 | 1.214 | 4.863 |
| | (0.00) | (0.615) | (0.912) | (1.441) | (3.881) |
| Tibialis | 0.00 | 0.699 | 1.042 | 0.384 | 0.816 |
| anterior ^{d} | (0.00) | (0.997) | (0.685) | (0.615) | (0.915) |
| Gastrocnemius ^{d} | 0.00 | 0.388 | 2.424 | 0.00 | 5.393 |
| | (0.00) | (0.573) | (2.329) | (0.00) | (2.695) |

^{a}Oligo = Oligonucleotide.
^{b}Conjugate doses are listed as mg/kg of anti-TfR1 Fab-oligonucleotide conjugate.
^{c}Oligo doses are listed as mg/kg oligonucleotide equivalent of the anti-TfR1 Fab-oligonucleotide dose.
^{d} Exon skipping values are mean % exon 51 skipping with standard deviations (n = 5) in parentheses.

Tissue oligonucleotide (oligo) accumulation was quantified using a hybridization ELISA with a probe complementary to the oligonucleotide sequence. A standard curve was generated and oligonucleotide levels (in ng/g) were derived from a linear regression of the standard curve. The oligonucleotide was distributed to all tissues evaluated at a higher level following the administration of the anti-TfR1 Fab-oligonucleotide conjugate as compared to the administration of unconjugated oligonucleotide (not covalently linked to antibody). Intravenous administration of unconjugated oligonucleotide resulted in levels of oligonucleotide that were close to background levels in all tissues evaluated at 2 and 4 weeks after the first dose was administered. Administration of the conjugate resulted in distribution of oligonucleotide through the tissues evaluated with a rank order of heart>diaphragm>bicep>quadriceps>gastrocnemious>tibialis anterior 2 weeks after first dosing.

The duration of tissue concentration was also assessed. Oligonucleotide levels were detectable at 4 weeks post dose in all tissues (Table 5). This indicates that the anti-TfR1 Fab shown in Table 2 enabled cellular internalization of the conjugate into muscle cells in vivo, resulting in activity of the exon skipping oligonucleotide in the muscle cells.

TABLE 5
Tissue distribution of DMD exon 51 skipping
oligonucleotide in cynomolgus monkeys

| | Time | | | | |
|---|---|---|---|---|---|
| | 2 weeks | | | 4 weeks | |
| | | Oligo | Conju- | Oligo | Conju- |
| Group | Vehicle | alone^{a} | gate | alone^{a} | gate |
| Conjugate | 0 | n/a | 122 | n/a | 122 |
| Dose^{b} | | | | | |
| Oligo alone | 0 | 30 | 30 | 30 | 30 |
| Dose^{c} | | | | | |
| Quadriceps ^{d} | 0 | 696.8 | 2436 | 197 | 682 |
| | (59.05) | (868.15) | (954.0) | (134) | (281) |
| Diaphragm ^{d} | 0± | 580.02 | 6750 | 60 | 3131 |
| | (144.3) | (360.11) | (2256) | (120) | (1618) |
| Heart ^{d} | 0 | 1449 | 27138 | 943 | 30410 |
| | (396.03) | (1337) | (6315) | (1803) | (9247) |
| Biceps ^{d} | 0 | 615.63 | 2840 | 130 | 1326 |
| | (69.58) | (335.17) | (980.31) | (80) | (623) |
| Tibialis | 0 | 564.71 | 1591 | 169 | 1087 |
| anterior ^{d} | (76.31) | (327.88) | (253.50) | (110) | (514) |
| Gastrocnemius ^{d} | 0 | 705.47 | 2096 | 170 | 1265 |
| | (41.15) | (863.75) | (474.04) | (69) | (272) |

^{a}Oligo = Oligonucleotide.
^{b}Conjugate doses are listed as mg/kg of anti-TfR1 Fab-oligonucleotide conjugate.
^{c}Oligo doses are listed as mg/kg oligonucleotide or oligonucleotide equivalent of the anti-TfR1 Fab-oligonucleotide conjugate dose.
^{d} Oligo values are mean concentrations of oligonucleotide in tissue as ng/g with standard deviations (n = 5) in parentheses.

### Example 3. Peptide Mapping to Determine Conjugation Sites of Oligonucleotides in Anti-TfR1 Fab-Oligonucleotide Conjugates

Peptide mapping was performed to determine conjugation sites of anti-TfR1 conjugates comprising the anti-TfR1 Fab having the heavy chain and light chain sequences shown in Table 3 covalently linked (through lysine conjugation) via a linker comprising a Valine-Citrulline sequence to a dystrophin (DMD) exon 51-skipping oligonucleotide. The exon 51-skipping oligonucleotide is a phosphorodiamidate morpholino oligomer (PMO) and comprises the base sequence of SEQ ID NO: 21. The conjugates comprise a structure of:

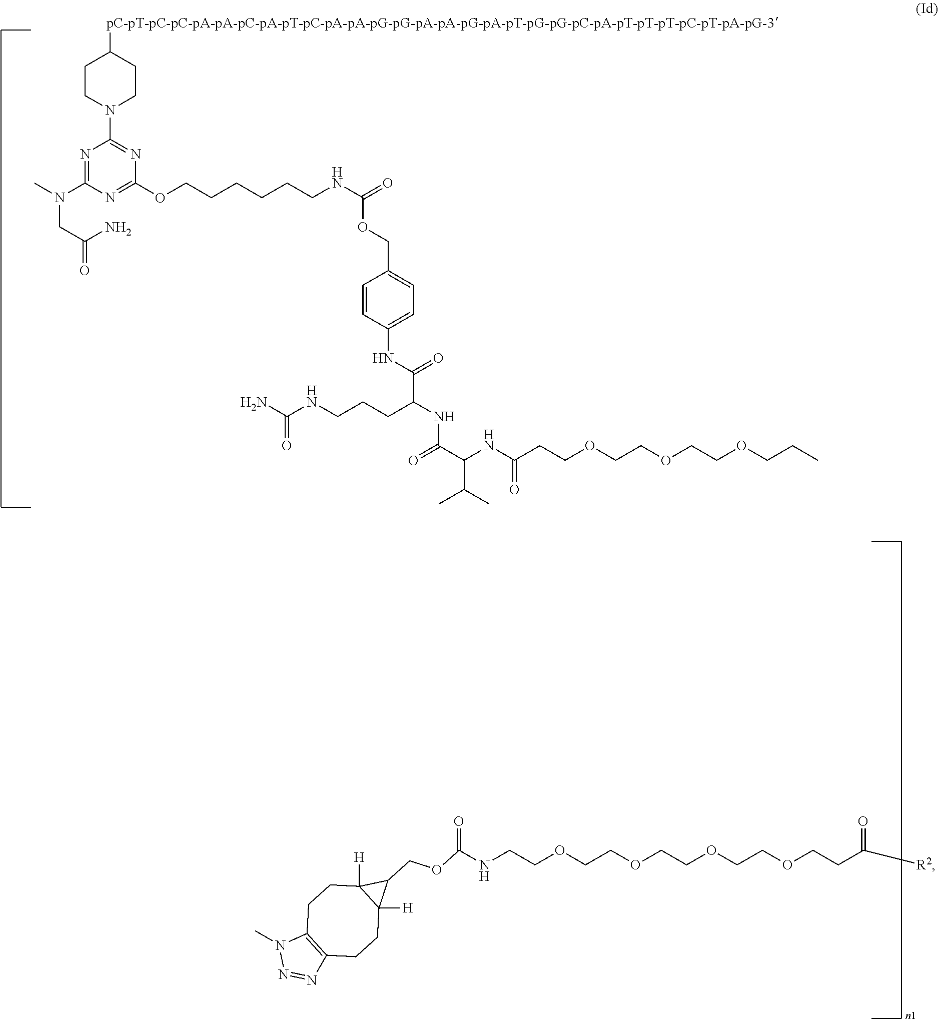

wherein R² is the anti-TfR1 Fab shown in Table 3, and wherein in each conjugate n1 is independently an integer of 1-5.

### Papain Digestion

The oligonucleotides were cleaved from the anti-TfR1 conjugates by digestion with papain. Thermo Scientific Immobilized Papain (P/N: 20341) was activated prior to digestion by adding 0.5 mL of the 50% slurry to 4 mL of freshly prepared Papain Activation Buffer (20 mM Sodium Phosphate, 10 mM EDTA, 20 mM Cysteine, pH 7.0). Papain resin was mixed thoroughly and centrifuged at 3000 rcf for 5 minutes to pull down the resin. The supernatant was discarded and the wash was repeated a second time. After two washes, the papain was resuspended to a 50% slurry by the addition of 250 μL Papain Activation Buffer. Anti-TfR1 conjugates underwent buffer exchange into Papain Digest buffer (20 mM Sodium Phosphate, 10 mM EDTA, pH 7.0) using Thermo Scientific Zeba desalting columns (P/N: 89882). The columns were equilibrated with 0.3 mL buffer 3 times prior to addition of conjugate. 40 μL of conjugate at a concentration of at least 8 mg/mL was added to 100 μL of equilibrated immobilized papain resin. Samples were incubated at 40° C. for 1 hour with shaking at 1400 rpm. After incubation, papain resin was removed by adding each sample to a filter tube and centrifuging briefly until all liquid sample was filtered through. Another set of Zeba columns were equilibrated with LC-MS grade water, 3×300 μL. 100 μL of papain-digested conjugate was added to each column and spun at 1500 rcf for 3 minutes to buffer exchange. 40 μg of material was diluted to a 50 μL volume with LC-MS water. Complete digestion of the oligonucleotide was observed in all samples.

### Chymotrypsin Digestion

The anti-TfR1 linker conjugates, with oligonucleotides cleaved, were digested with immobilized chymotrypsin. 15 μL of Thermo SMART Chymotrypsin resin (Kit P/N: 60109104MBLPH) was added to an Eppendorf tube. 150 μL of Chymotrypsin Smart Digest Buffer was added. 50 μL of the anti-TfR1 linker conjugate sample prepared after papain digest was added. A heat block was set to 70° C. and 1400 rpm mix, and incubated for 2.5 hours. After incubation, the papain resin was removed by adding each sample to a filter tube and centrifuging briefly until all liquid sample is filtered through. A stock solution of 500 mM tris(2-carboxyethyl)phosphine (TCEP) in water was prepared. The stock solution was added to the digest sample to a final concentration of 20 mM TCEP, and was incubated for 30 mins at room temp to reduce disulfides. Samples were concentrated by speed vac until the total sample volume was about 100 μL. 25 μL of peptide sample (about 10 ug total peptide) was then injected into the mass spectrometer for peptide mapping analysis.

A summary of the percent occupancy of oligonucleotide per linker site is shown below in Table 6. The hot spot linker sites were lysine 188 (K188) and lysine 190 (K190) in the light chain constant regions in the composition based on Kabat numbering. Collectively, about 93% of lysine residues corresponding to K188 and K190 were covalently linked to an oligonucleotide. Another major linker site was lysine 213 (K213) in the heavy chain constant regions in the compositions, as about 19.6% of lysine residues corresponding to K213 were covalently linked to an oligonucleotide.

TABLE 6

| Site** | % Occupancy per Linker Site |
|---|---|
| Heavy chain Variable | ~7% |
| region K13 | |
| Heavy chain Constant | ~3% |
| region K117 | |
| Heavy chain Constant | ~19.6% |
| region K213 | |
| Light chain Variable | ~3% |
| region K39 | |
| Light chain Constant | ~5% |
| region K126 | |
| Light chain Constant | ~4% |
| region K149 | |
| Light chain Constant | ~93% (includes complexes in which |
| region K188 | K188 is linked, in which K189 is |
| Light chain Constant | linked, and in which both K188 |
| region K190 | and K190 are linked)* |

*hot spot is either K188 or K190
**based on Kabat numbering

### Example 4. Peptide Mapping to Determine Conjugation Sites of Oligonucleotides on Anti-TfR1 Fab-Oligonucleotide Conjugates

Peptide mapping was performed to determine conjugation sites of anti-TfR1 conjugates comprising the anti-TfR1 Fab having the heavy chain and light chain sequences shown in Table 2 covalently linked (through lysine conjugation) via a linker comprising a Valine-Citrulline sequence to a dystrophin (DMD) exon 51-skipping oligonucleotide. The exon 51-skipping oligonucleotide is a phosphorodiamidate morpholino oligomer (PMO) and comprises the base sequence of SEQ ID NO: 21. The conjugates comprise a structure of:

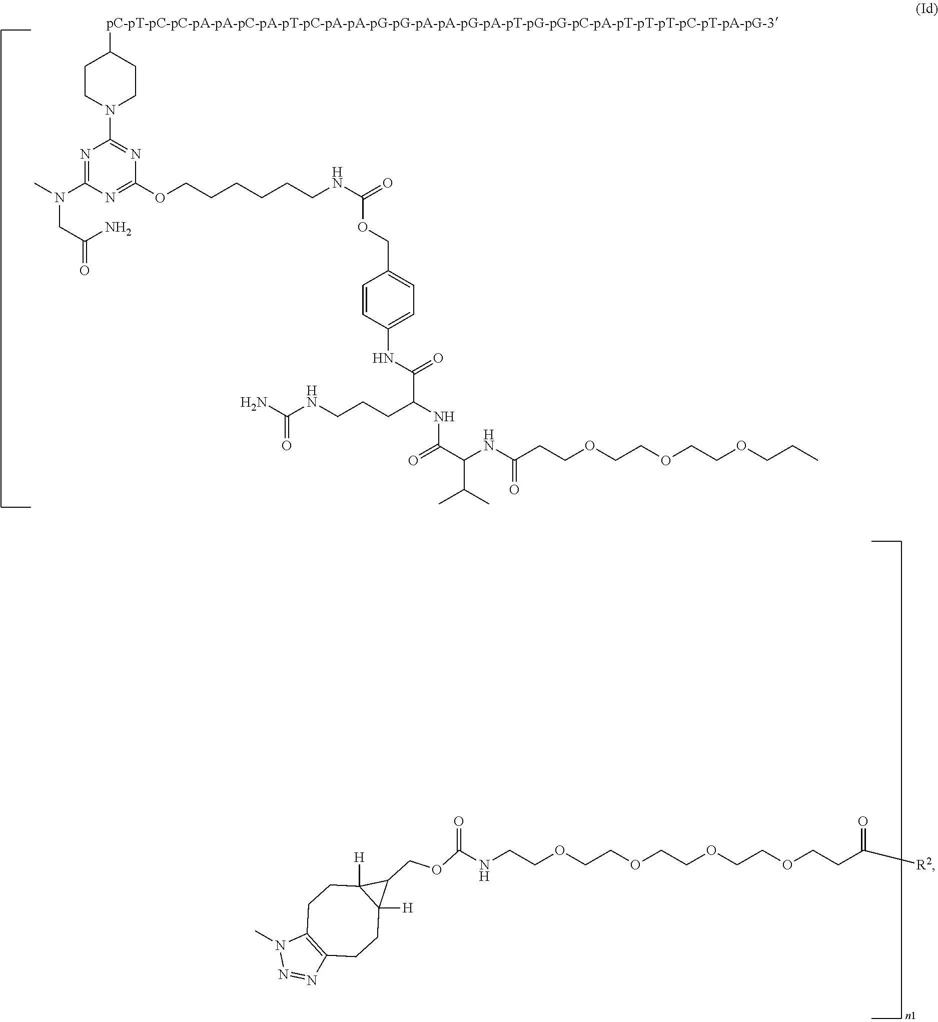

wherein R² is the anti-TfR1 Fab shown in Table 2, and wherein in each conjugate n1 is independently an integer of 1-5.

### Papain Digestion

The oligonucleotides were cleaved from the anti-TfR1 conjugates by digestion with papain. Thermo Scientific Immobilized Papain (P/N: 20341) was activated prior to digestion by adding 0.5 mL of the 50% slurry to 4 mL of freshly prepared Papain Activation Buffer (20 mM Sodium Phosphate, 10 mM EDTA, 20 mM Cysteine, pH 7.0). Papain resin was mixed thoroughly and centrifuged at 3000 rcf for 5 minutes to pull down the resin. The supernatant was discarded and the wash was repeated a second time. After two washes, the papain was resuspended to a 50% slurry by the addition of 250 μL Papain Activation Buffer. Anti-TfR1 conjugates underwent buffer exchange into Papain Digest buffer (20 mM Sodium Phosphate, 10 mM EDTA, pH 7.0) using Thermo Scientific Zeba desalting columns (P/N: 89882). The columns were equilibrated with 0.3 mL buffer 3 times prior to addition of conjugate. 40 μL of conjugate at a concentration of at least 8 mg/mL was added to 100 μL of equilibrated immobilized papain resin. Samples were incubated at 40° C. for 1 hour with shaking at 1400 rpm. After incubation, papain resin was removed by adding each sample to a filter tube and centrifuging briefly until all liquid sample was filtered through. Another set of Zeba columns were equilibrated with LC-MS grade water, 3×300 μL. 100 μL of papain-digested conjugate was added to each column and spun at 1500 rcf for 3 minutes to buffer exchange. 40 μg of material was diluted to a 50 μL volume with LC-MS water. Complete digestion of the oligonucleotide was observed in all samples.

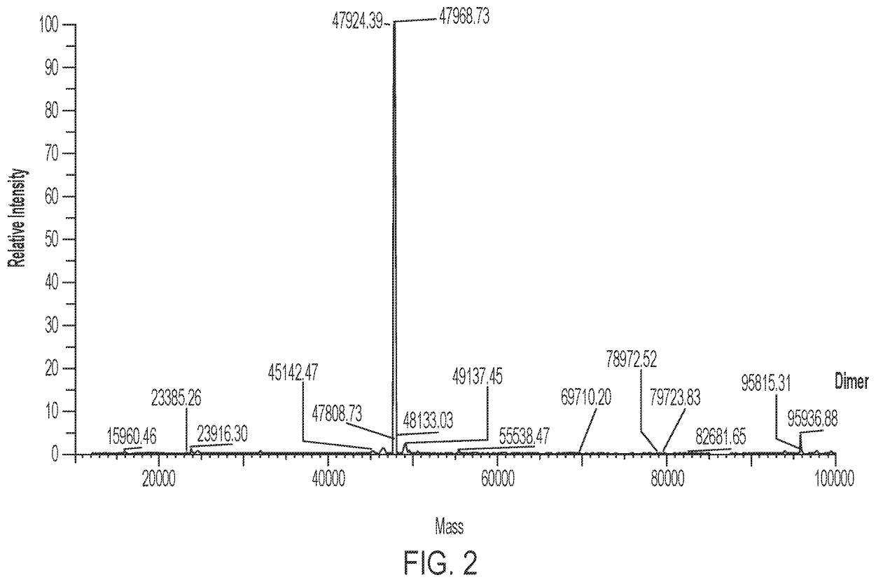

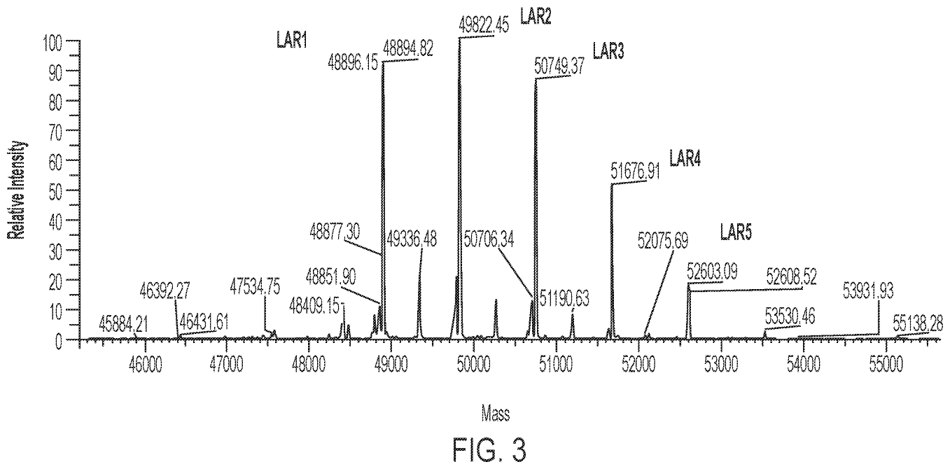

A mass spectrum of intact mass of the unlinked anti-TfR1 antibody is shown in FIG. 2. The intact mass of the unlinked antibody is 47968.73 Da as determined by mass spectrometry and is consistent with the theoretical mass of the antibody with a post-translational modification (PTM) that converted the first residue (Q) of the heavy chain to a pyroglutamate, which is 47968.01 Da (the theoretical mass of the antibody without the PTM is 47924.39 Da). A mass spectrum showing masses of anti-TfR1 antibody-linker complexes of the compositions provided herein after cleavage of the oligonucleotides via papain digestion is shown in FIG. 3. Five major peaks were observed and each corresponds to a linker to antibody ratio (LAR) of 1-5. Next to each peak, the mass of the anti-TfR1 antibody-linker complex is shown, which equals the intact antibody (with a pyroglutamate PTM) mass (47968.01 Da) plus the linker mass (˜927.07 Da per linker). The experimental masses of the complexes determined by mass spectrometry are shown in FIG. 3. The anti-TfR1 antibody-linker complex with an LAR of 1 has an experimental mass of 48894.82, which is consistent with a theoretical mass of 48895.08. The anti-TfR1 antibody-linker complex with an LAR of 2 has an experimental mass of 49822.45, which is consistent with a theoretical mass of 49822.15. The anti-TfR1 antibody-linker complex with an LAR of 3 has an experimental mass of 50749.37, which is consistent with a theoretical mass of 50749.22. The anti-TfR1 antibody-linker complex with an LAR of 4 has an experimental mass of 51676.91, which is consistent with a theoretical mass of 51676.29. The anti-TfR1 antibody-linker complex with an LAR of 5 has an experimental mass of 52603.09, which is consistent with a theoretical mass of 52603.36

### Chymotrypsin Digestion

The anti-TfR1 linker conjugates, with oligonucleotides cleaved, were digested with immobilized chymotrypsin. 15 μL of Thermo SMART Chymotrypsin resin (Kit P/N: 60109104MBLPH) was added to an Eppendorf tube. 150 μL of Chymotrypsin Smart Digest Buffer was added. 50 μL of the anti-TfR1 linker conjugate sample prepared after papain digest was added. A heat block was set to 70° C. and 1400 rpm mix, and incubated for 2.5 hours. After incubation, the papain resin was removed by adding each sample to a filter tube and centrifuging briefly until all liquid sample is filtered through. A stock solution of 500 mM tris(2-carboxyethyl)phosphine (TCEP) in water was prepared. The stock solution was added to the digest sample to a final concentration of 20 mM TCEP, and was incubated for 30 mins at room temp to reduce disulfides. Samples were concentrated by speed vac until the total sample volume was about 100 μL. 25 μL of peptide sample (about 10 ug total peptide) was then injected into the mass spectrometer for peptide mapping analysis. There was 100% sequence coverage in all chymotrypsin digests (data not shown) and papain-cleaved linkers were identified in all conjugate groups. Oligonucleotides were then quantified per heavy and light chains of the antibodies.

A summary of the percent occupancy of oligonucleotide per linker site is shown below in Table 7. The hot spot linker sites were lysine 188 (K188) and lysine 190 (K190) in the light chain constant regions in the composition based on Kabat numbering. Collectively, 85-95% of lysine residues corresponding to K188 and K190 were covalently linked to an oligonucleotide. Another major linker site was lysine 213 (K213) in the heavy chain constant regions in the compositions, as 15-20% of lysine residues corresponding to K213 were covalently linked to an oligonucleotide.

TABLE 7

| Site** | % Occupancy per Linker Site |
|---|---|
| Heavy Variable K13 | ~5% |
| Heavy Variable K43 | ~2% |
| Heavy Variable K64 | N/A |
| Heavy Variable K81 | ~2-5% |
| Heavy Constant K117 | N/A |
| Heavy Constant K213 | ~15-40% |
| Heavy Constant K218 | N/A |
| Heavy Constant | ~10% |
| K221/K222 | |
| Heavy Constant K228 | N/A |
| Light Variable K39 | ~2% |
| Light Variable K107 | ~5% |
| Light Constant K126 | ~5% |
| Light Constant K145 | ~5% |
| Light Constant K188 | 85-95% (includes complexes in which |
| Light Constant K190 | K188 is linked, in which K189 is |
| | linked, and in which both K188 |
| | and K190 are linked)* |
| Light Constant K207 | 3% |

*hot spot is either K188 or K190
**based on Kabat numbering

### ADDITIONAL EMBODIMENTS

1. A composition comprising a plurality of complexes, wherein each complex comprises an antibody covalently linked to one or more oligonucleotides, each oligonucleotide being covalently linked at a linkage site represented by a lysine (K) residue of the antibody,
   wherein the antibody comprises a heavy chain comprising a heavy chain variable region (VH) and a heavy chain constant region, and a light chain comprising a light chain variable region (VL) and a light chain constant region,
   and wherein, at least 80% of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody.
2. The composition of embodiment 1, wherein at least 15% of the heavy chain constant regions of the antibodies of the complexes in the composition are independently covalently linked to an oligonucleotide at a linkage site represented by K213 (based on Kabat numbering) of the heavy chain constant region of each antibody.
3. A composition comprising a plurality of complexes, wherein each complex comprises an antibody covalently linked to one or more oligonucleotides, each oligonucleotide being covalently linked at a linkage site represented by a lysine (K) residue of the antibody,
   wherein the antibody comprises a heavy chain comprising a heavy chain variable region (VH) and a heavy chain constant region, and a light chain comprising a light chain variable region (VL) and a light chain constant region,
   wherein at least 80% of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody.
4. The composition of embodiment 3, wherein at least 15% of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody.
5. The composition of any one of embodiments 1-4, wherein the antibody is an anti-transferrin receptor (TfR1) antibody comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16.
6. The composition of any one of embodiments 1-4, wherein the antibody is an anti-transferrin receptor (TfR1) antibody comprising: a heavy chain complementarity determining region 1 (CDR-H1) comprising a sequence as set forth in SEQ ID NOs: 37, 43, or 48, a heavy chain complementarity determining region 2 (CDR-H2) comprising a sequence as set forth in SEQ ID NOs: 38, 44, or 49, a heavy chain complementarity determining region 3 (CDR-H3) comprising a sequence as set forth in SEQ ID NOs: 39, 45, or 50, a light chain complementarity determining region 1 (CDR-L1) comprising a sequence as set forth in SEQ ID NOs: 40, 46, 51, a light chain complementarity determining region 2 (CDR-L2) comprising a sequence as set forth in SEQ ID NOs: 41 or 47, and a light chain complementarity determining region 3 (CDR-L3) comprising a sequence as set forth in SEQ ID NOs: 42 or 52.
7. The composition of any one of embodiments 1-6, wherein oligonucleotide is a phosphorodiamidate morpholino oligomer (PMO).
8. A composition comprising a plurality of complexes of the formula (I): [R¹]ₙ₁—R², wherein
each R¹ independently comprises a group of the formula (Ia):

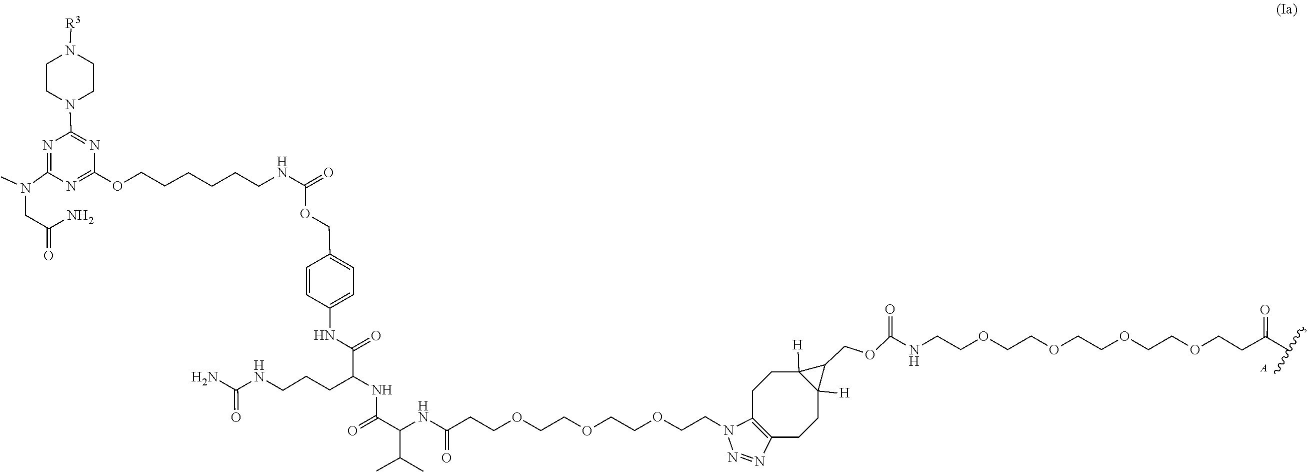

R² comprises an anti-transferrin receptor (TfR1) antibody comprising a heavy chain comprising a heavy chain variable region (VH) and a heavy chain constant region, and a light chain comprising a light chain variable region (VL) and a light chain constant region; and wherein the anti-TfR1 antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16;
R³ comprises a phosphorodiamidate morpholino oligomer (PMO) comprising a nucleobase sequence of CTCCAACATCAAGGAAGATGGCATTTCTAG (SEQ ID NO: 21);
wherein each R¹ is covalently linked at attachment point A to R² via a linkage site represented by a lysine (K) residue of the anti-TfR1 antibody, and wherein, at least 80% of the light chain constant regions of the anti-TfR1 antibodies of the complexes in the composition are independently covalently linked to R¹ at a linkage site represented by K188 (based on Kabat numbering) and/or a linkage site represented by K190 (based on Kabat numbering) of the light chain constant region of each antibody; and
wherein in each complex, n1 is independently an integer of one or greater representing the number of instances of R¹, optionally wherein the average value of n1 of complexes in the composition is in the range of 1 to 5.
9. A composition comprising a plurality of complexes of the formula (I): [R¹]ₙ₁—R², wherein
each R¹ independently comprises a group of the formula (Ib):

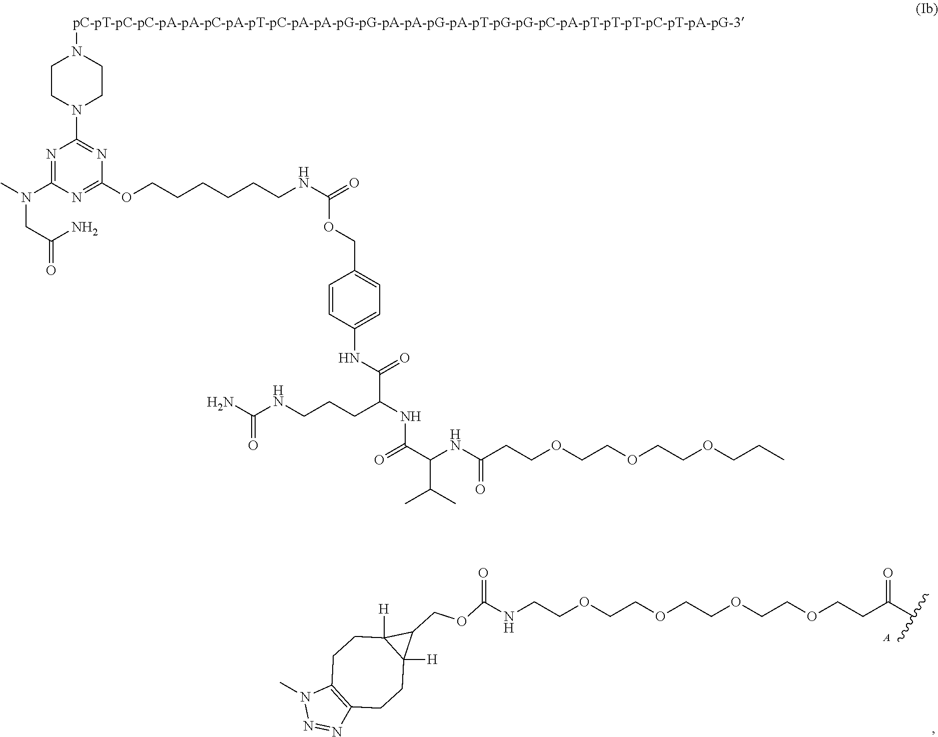

in which -p is a phosphorodiamidate linkage of a phosphorodiamidate morpholino oligomer (PMO), and wherein the PMO comprises a base sequence of CTCCAACATCAAGGAAGATGGCATTTCTAG (SEQ ID NO: 21);
   R² comprises an anti-transferrin receptor (TfR1) antibody comprising a heavy chain comprising a heavy chain variable region (VH) and a heavy chain constant region, and a light chain comprising a light chain variable region (VL) and a light chain constant region; and
wherein the anti-TfR1 antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16; and
   wherein each R¹ is covalently linked at attachment point A to R² via a linkage site represented by a lysine (K) residue of the anti-TfR1 antibody, and wherein, at least 80% of the light chain constant regions of the anti-TfR1 antibodies of the complexes in the composition are independently covalently linked to R¹ at a linkage site represented by K188 (based on Kabat numbering) and/or a linkage site represented by K190 (based on Kabat numbering) of the light chain constant region of each antibody; and
   wherein in each complex, n1 is independently an integer of one or greater representing the number of instances of R¹, and optionally wherein the average value of n1 of the complexes of the composition is in the range of 1 to 5.
10. A composition comprising a plurality of complexes of the formula (I): [R¹]ₙ₁—R², wherein: each R¹ comprises a group of the formula (Ic):

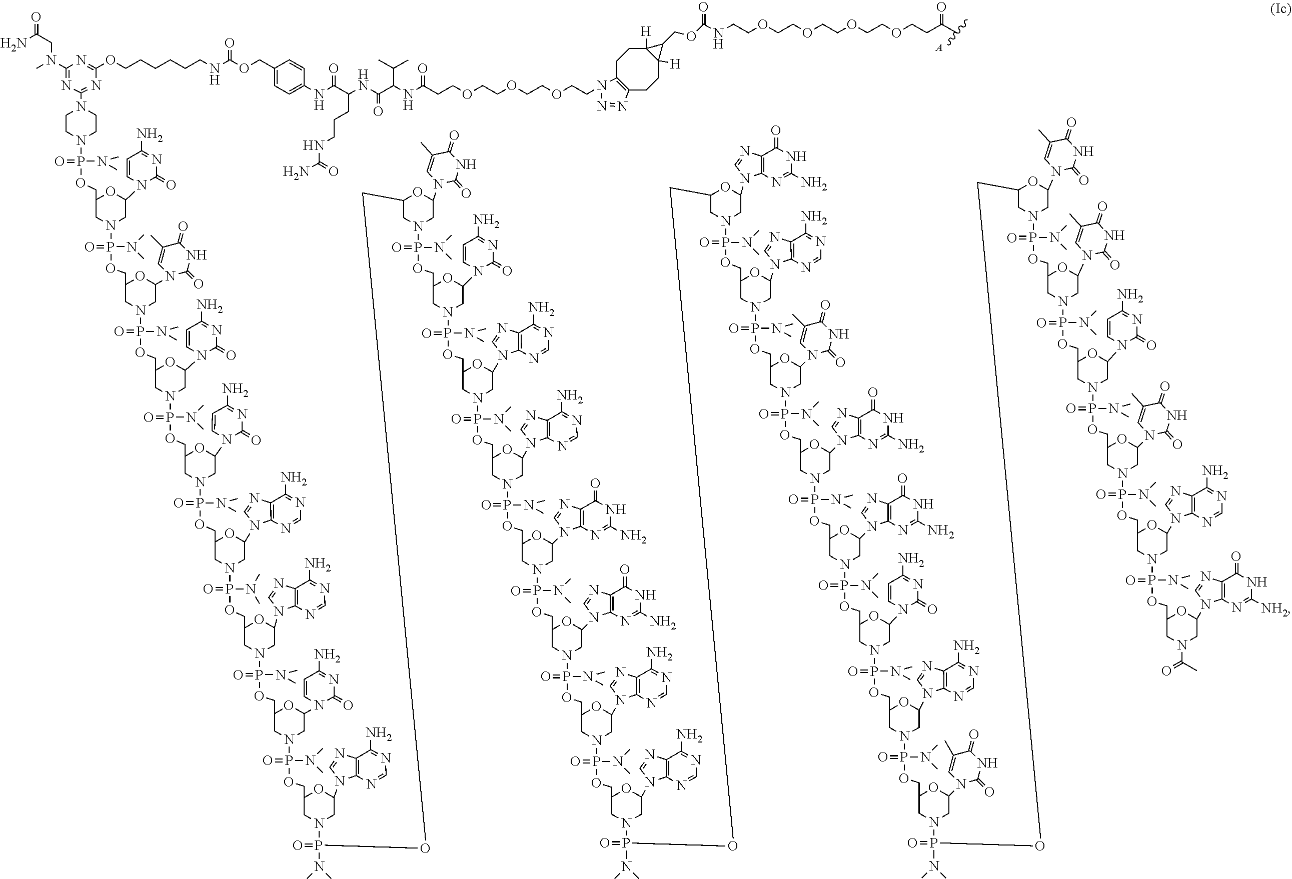

R² comprises an anti-transferrin receptor (TfR1) antibody comprising a heavy chain comprising a heavy chain variable region (VH) and a heavy chain constant region, and a light chain comprising a light chain variable region (VL) and a light chain constant region; and wherein the anti-TfR1 antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16; and
wherein each R¹ is covalently linked at attachment point A to R² via a linkage site represented by a lysine (K) residue of the anti-TfR1 antibody, and wherein, at least 80% of the light chain constant regions of the anti-TfR1 antibodies of the complexes in the composition are independently covalently linked to R¹ at a linkage site represented by K188 (based on Kabat numbering) and/or a linkage site represented by K190 (based on Kabat numbering) of the light chain constant region of each antibody; and
wherein in each complex, n1 is independently an integer of one or greater representing the number of instances of R¹, and optionally wherein the average value of n1 of the complexes of the composition is in the range of 1 to 5.
11. The composition of any one of embodiments 8-10, wherein 85%-95% of the light chain constant regions of the anti-TfR1 antibodies of the complexes in the composition are independently covalently linked to R¹ at a linkage site represented by K188 (based on Kabat numbering) and/or a linkage site represented by K190 (based on Kabat numbering) of the light chain constant region of each antibody.
12. The composition of any one of embodiments 8-11, wherein 90-95% of the light chain constant regions of the anti-TfR1 antibodies of the complexes in the composition are independently covalently linked to R¹ at a linkage site represented by K188 (based on Kabat numbering) and/or a linkage site represented by K190 (based on Kabat numbering) of the light chain constant region of each antibody.
13. The composition of any one of embodiments 8-12, wherein at least 15% of the heavy chain constant regions of the anti-TfR1 antibodies of the complexes in the composition are independently covalently linked to R¹ at a linkage site represented by K213 (based on Kabat numbering) of the heavy chain constant region of each antibody.
14. The composition of any one of embodiments 8-13, wherein 15-45% of the heavy chain constant regions of the anti-TfR1 antibodies of the complexes in the composition are independently covalently linked to R¹ at a linkage site represented by K213 (based on Kabat numbering) of the heavy chain constant region of each antibody.
15. The composition of any one of embodiments 1-14, wherein the antibody is a Fab fragment, a full-length IgG, a Fab′ fragment, or a F(ab′)2 fragment.
16. The composition of any one of embodiments 1-15, wherein the antibody is a Fab fragment.
17. The composition of any one of embodiments 1-16, wherein the VH comprises an amino acid sequence at least 85% identical to SEQ ID NO: 17; and/or wherein the VL comprises an amino acid sequence at least 85% identical to SEQ ID NO: 18.
18. The composition of any one of embodiments 1-17, wherein the antibody comprises a heavy chain variable region (VH) comprising the amino acid sequence of SEQ ID NO: 17 and a light chain variable region (VL) comprising the amino acid sequence of SEQ ID NO: 18.
19. The composition of any one of embodiments 1-18, wherein the heavy chain comprises the amino acid sequence of SEQ ID NO: 19 and a light chain comprising the amino acid sequence of SEQ ID NO: 20.
20. A method of promoting expression or activity of a dystrophin protein in a subject, comprising administering to the subject an effective amount of the composition of any one of embodiments 1-19.
21. A method of treating Duchenne Muscular Dystrophy (DMD) in a subject, comprising administering to the subject an effective amount of the composition of any one of embodiments 1-19.
22. The method of embodiment 20 or embodiment 21, wherein the subject has a mutated dystrophin allele comprising a mutation amenable to exon 51 skipping.
23. The method of embodiment 22, wherein the mutated dystrophin allele comprises a frameshift mutation in exon 51.
24. The method of any one of embodiments 20-23, wherein the complexes promote expression or activity of dystrophin protein in the subject.
25. The method of embodiment 24, wherein the dystrophin protein is a truncated dystrophin protein.
26. A method of determining a drug to antibody ratio (DAR) range of a first plurality of complexes, each complex comprising an antibody covalently linked to one or more oligonucleotides via a linker, wherein each linker comprises one or more protease cleavage sites, the method comprising:
   (i) removing the one or more oligonucleotides from the antibodies to which they are covalently linked of the first plurality of complexes by cleaving at least one of the one or more protease cleavage sites of the linker, wherein cleaving at least one of the one or more protease cleavage sites of the linker results in a second plurality of complexes, each complex comprising an antibody covalently linked to one or more partial linkers;
   (ii) obtaining the second plurality of complexes resulting from step (i);
   (iii) determining the masses of the complexes obtained in step (ii) via mass spectrometry; and
   (iv) determining the DAR range of the complexes obtained in step (ii); wherein a detected mass by mass spectrometry corresponding to the mass of the antibody plus the mass of n1 partial linkers indicates a DAR of n1, wherein n1 is an integer of one or greater.
27. A method of analyzing a first plurality of complexes, each complex comprising an antibody covalently linked to one or more oligonucleotides via a linker, wherein each linker comprises one or more protease cleavage sites, the method comprising:
   (i) removing the one or more oligonucleotides from the antibodies to which they are covalently linked of the first plurality of complexes by cleaving at least one of the one or more protease cleavage sites in the linker, wherein cleaving at least one of the one or more protease cleavage sites in the linker results in a second plurality of complexes, each complex comprising an antibody covalently linked to one or more partial linkers, and wherein the antibody remains intact;
   (ii) obtaining the second plurality complexes resulting from step (i);
   (iii) digesting the antibodies of complexes obtained in (ii) with a protease to obtain fragments of the antibodies; and
   (iv) determining the mass of the fragments of the antibodies obtained in step (iii) via mass spectrometry to identify the fragments covalently linked one or more partial linkers.
28. The method of embodiment 26 or embodiment 27, wherein each complex of the first plurality of complexes comprises a structure of the formula (I): [R¹]ₙ₁—R², wherein: each R¹ comprises a group of the formula (Ic):

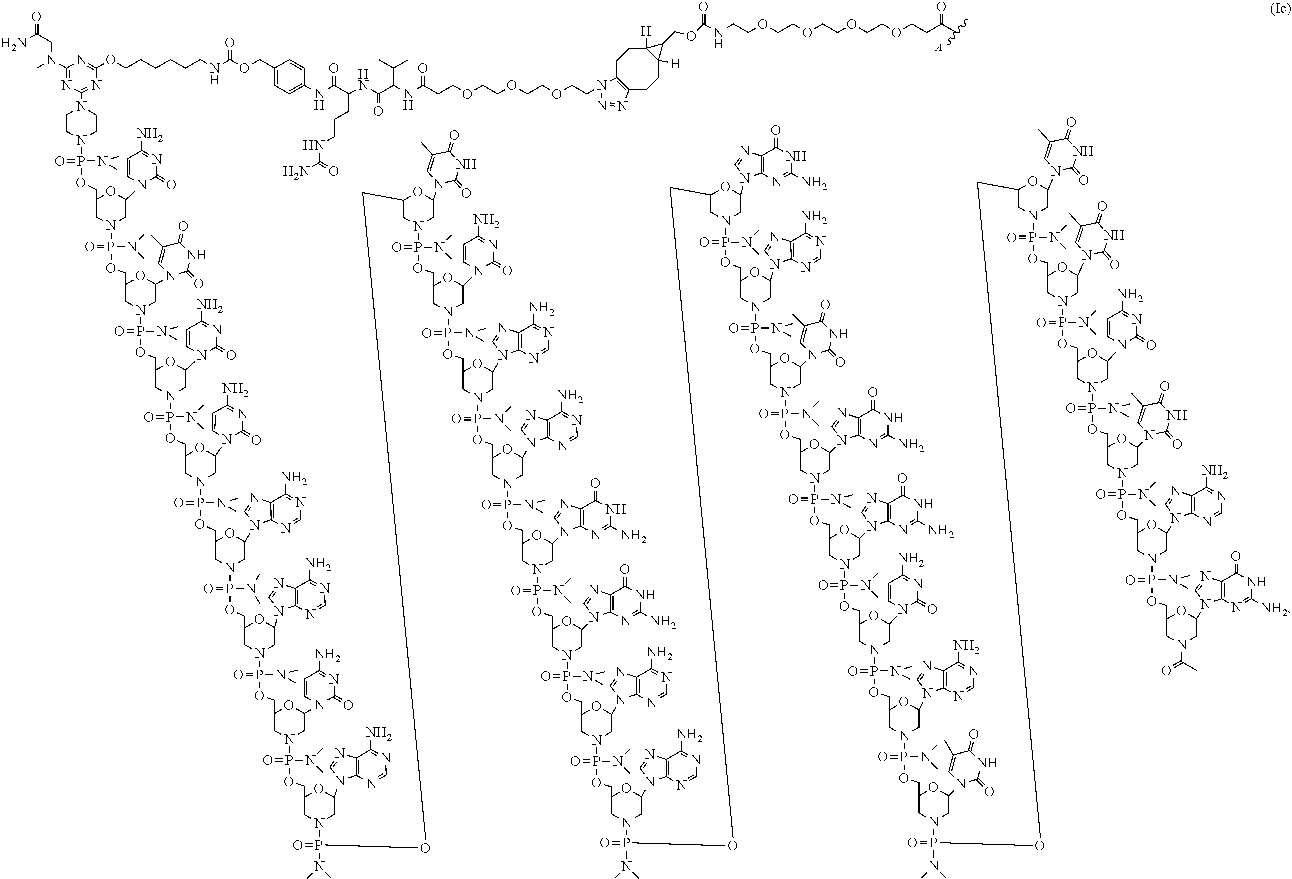

R² comprises an anti-transferrin receptor (TfR1) antibody comprising a heavy chain comprising a heavy chain variable region (VH) and a heavy chain constant region, and a light chain comprising a light chain variable region (VL) and a light chain constant region; and wherein the anti-TfR1 antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16; and
wherein each R¹ is covalently linked at attachment point A to R² via a linkage site represented by a lysine (K) residue of the anti-TfR1 antibody, and wherein, at least 80% of the light chain constant regions of the anti-TfR1 antibodies of the complexes in the composition are independently covalently linked to R¹ at a linkage site represented by K188 (based on Kabat numbering) and/or a linkage site represented by K190 (based on Kabat numbering) of the light chain constant region of each antibody; and
wherein in each complex, n1 is independently an integer of one or greater representing the number of instances of R¹, and optionally wherein the average value of n1 of the complexes of the composition is in the range of 1 to 5.
29. The method of any one of embodiments 26-28, wherein the cleaving of step (i) is carried out with papain.
30. The method of any one of embodiments 27-29, wherein the digesting of step (iii) is carried out with a chymotrypsin.

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or (e.g., and) one or more modified nucleotides and/or (e.g., and) one or more modified internucleoside linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

What is claimed is:

1. A composition comprising a plurality of complexes, wherein each complex comprises an antibody covalently linked to one or more oligonucleotides, each oligonucleotide being covalently linked at a linkage site represented by a lysine (K) residue of the antibody,
wherein the antibody comprises a heavy chain comprising a heavy chain variable region (VH) and a heavy chain constant region, and a light chain comprising a light chain variable region (VL) and a light chain constant region,
and wherein, at least 80% of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant region of each antibody.

2. The composition of claim 1, wherein at least 15% of the heavy chain constant regions of the antibodies of the complexes in the composition are independently covalently linked to an oligonucleotide at a linkage site represented by K213 (based on Kabat numbering) of the heavy chain constant region of each antibody.

3. A composition comprising a plurality of complexes, wherein each complex comprises an antibody covalently linked to one or more oligonucleotides, each oligonucleotide being covalently linked at a linkage site represented by a lysine (K) residue of the antibody,
wherein the antibody comprises a heavy chain comprising a heavy chain variable region (VH) and a heavy chain constant region, and a light chain comprising a light chain variable region (VL) and a light chain constant region,
wherein at least 80% of light chain constant regions of the antibodies of the complexes in the composition are independently covalently linked to an oligonucleotide at linkage sites represented by lysine (K) residues in a sequence motif DYEKHKVYA (SEQ ID NO: 53) of the light chain constant region of each antibody.

4. The composition of claim 3, wherein at least 15% of the heavy chain constant regions of the antibodies of the complexes in the composition are independently covalently linked to an oligonucleotide at a linkage site represented by the lysine (K) residue in a sequence motif VNHKPSN (SEQ ID NO: 58) of the heavy chain constant region of each antibody.

5. The composition of any one of claims 1-4, wherein the antibody is an anti-transferrin receptor (TfR1) antibody comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16.

6. The composition of any one of claims 1-5, wherein oligonucleotide is a phosphorodiamidate morpholino oligomer (PMO).

7. A composition comprising a plurality of complexes of the formula (I): [R¹]ₙ₁—R², wherein
each R¹ independently comprises a group of the formula (Ia): 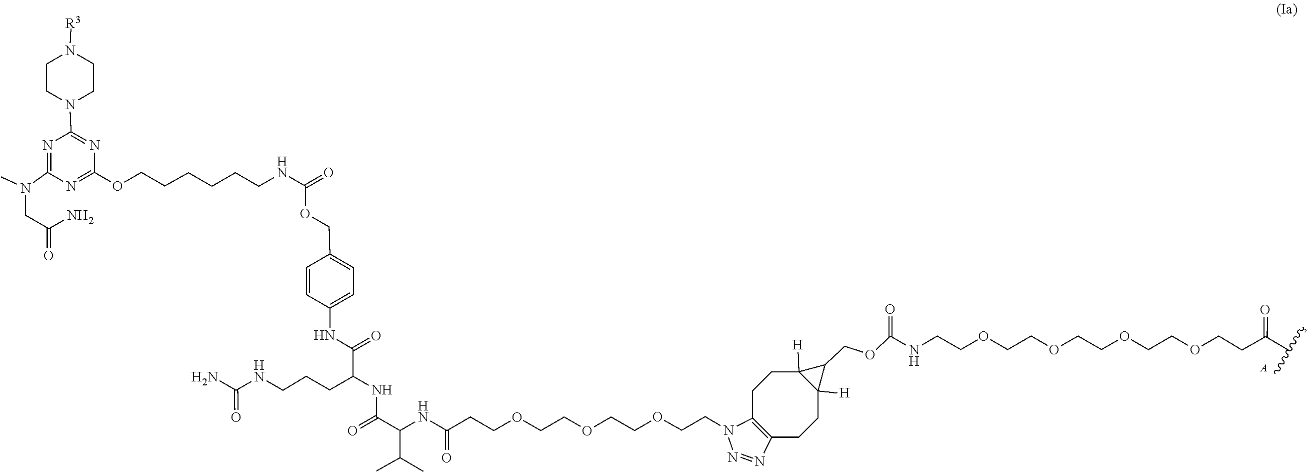
R² comprises an anti-transferrin receptor (TfR1) antibody comprising a heavy chain comprising a heavy chain variable region (VH) and a heavy chain constant region, and a light chain comprising a light chain variable region (VL) and a light chain constant region; and wherein the anti-TfR1 antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16;
R³ comprises a phosphorodiamidate morpholino oligomer (PMO) comprising a nucleobase sequence of CTCCAACATCAAGGAAGATGGCATTTCTAG (SEQ ID NO: 21);
wherein each R¹ is covalently linked at attachment point A to R² via a linkage site represented by a lysine (K) residue of the anti-TfR1 antibody, and wherein, at least 80% of the light chain constant regions of the anti-TfR1 antibodies of the complexes in the composition are independently covalently linked to R¹ at a linkage site represented by K188 (based on Kabat numbering) and/or a linkage site represented by K190 (based on Kabat numbering) of the light chain constant region of each antibody; and
wherein in each complex, n1 is independently an integer of one or greater representing the number of instances of R¹, optionally wherein the average value of n1 of complexes in the composition is in the range of 1 to 5.

8. A composition comprising a plurality of complexes of the formula (I): [R₁]ₙ₁—R², wherein
each R¹ independently comprises a group of the formula (Ib): 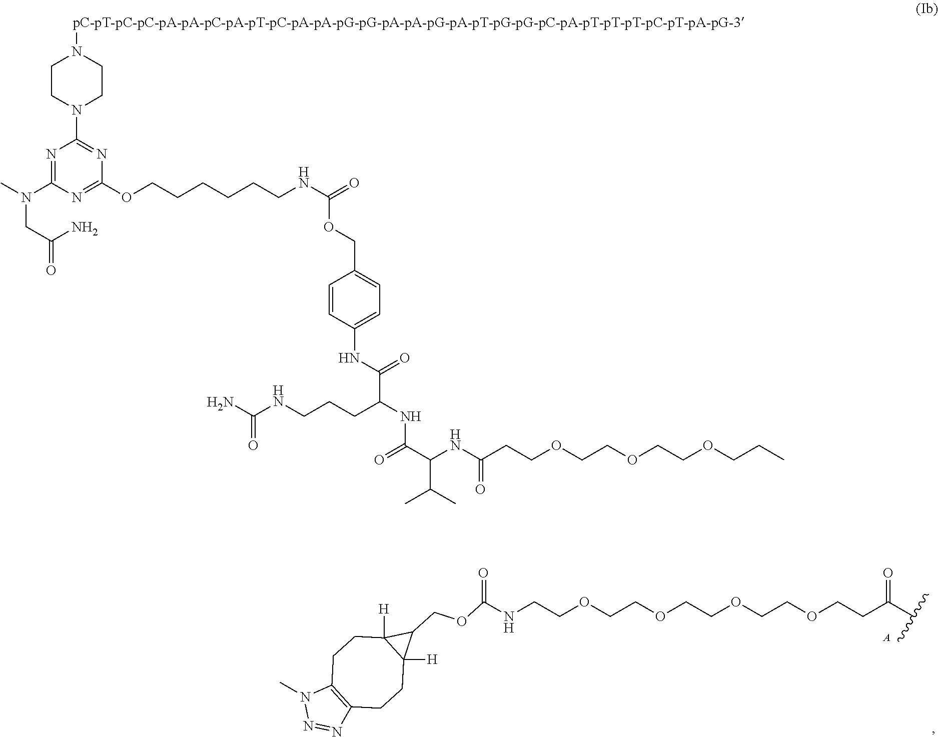
in which -p is a phosphorodiamidate linkage of a phosphorodiamidate morpholino oligomer (PMO), and wherein the PMO comprises a base sequence of CTCCAACATCAAGGAAGATGGCATTTCTAG (SEQ ID NO: 21);
R² comprises an anti-transferrin receptor (TfR1) antibody comprising a heavy chain comprising a heavy chain variable region (VH) and a heavy chain constant region, and a light chain comprising a light chain variable region (VL) and a light chain constant region; and wherein the anti-TfR1 antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16; and
wherein each R¹ is covalently linked at attachment point A to R² via a linkage site represented by a lysine (K) residue of the anti-TfR1 antibody, and wherein, at least 80% of the light chain constant regions of the anti-TfR1 antibodies of the complexes in the composition are independently covalently linked to R¹ at a linkage site represented by K188 (based on Kabat numbering) and/or a linkage site represented by K190 (based on Kabat numbering) of the light chain constant region of each antibody; and
wherein in each complex, n1 is independently an integer of one or greater representing the number of instances of R¹, and optionally wherein the average value of n1 of the complexes of the composition is in the range of 1 to 5.

9. A composition comprising a plurality of complexes of the formula (I): [R¹]ₙ₁—R², wherein: each R¹ comprises a group of the formula (Ic): 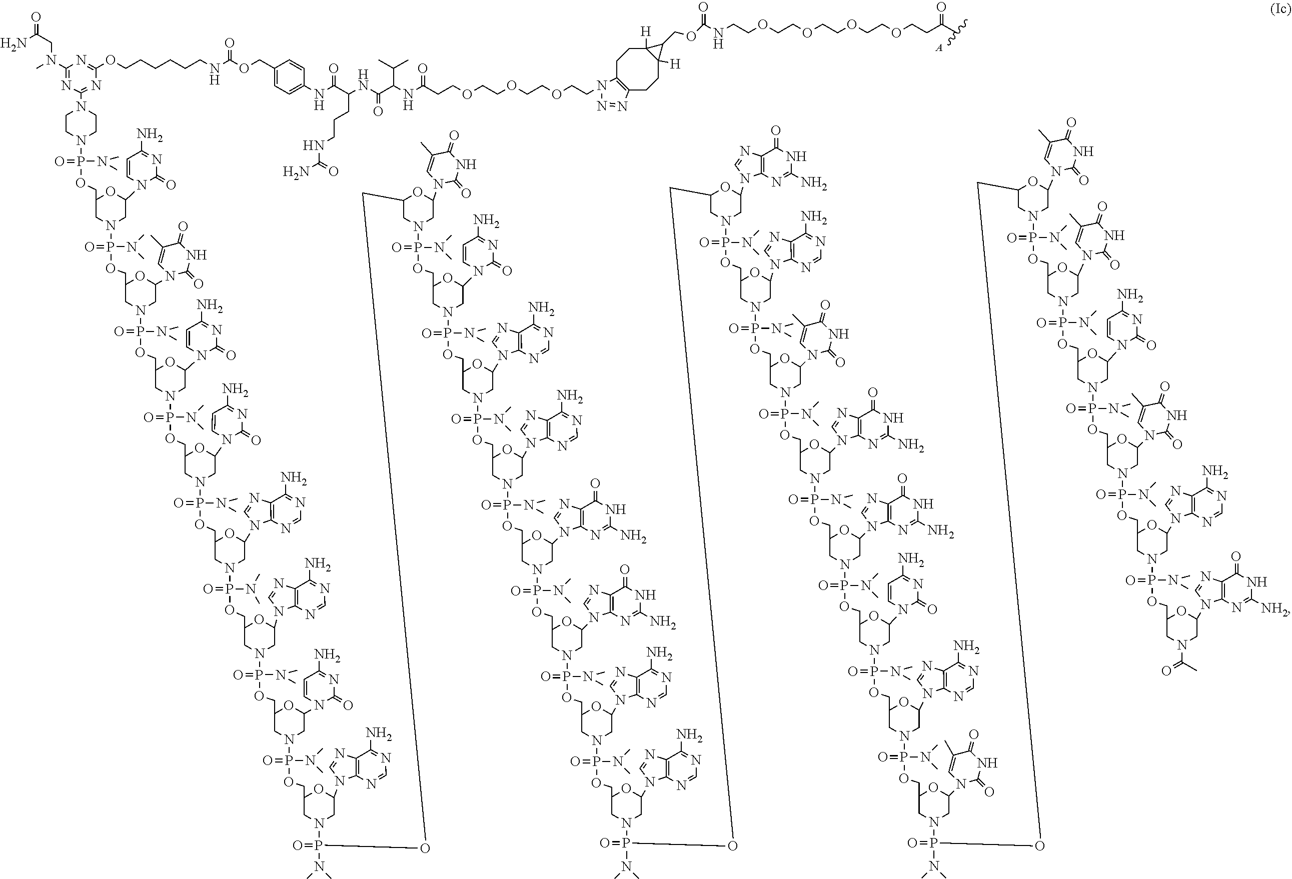
R² compromises an anti-transferrin receptor (TfR1) antibody comprising a heavy chain comprising a heavy chain variable region (VH) and a heavy chain constant region, and a light chain comprising a light chain variable region (VL) and a light chain constant region; and wherein the anti-TfR1 antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16; and
wherein each R¹ is covalently linked at attachment point A to R² via a linkage site represented by a lysine (K) residue of the anti-TfR1 antibody, and wherein, at least 80% of the light chain constant regions of the anti-TfR1 antibodies of the complexes in the composition are independently covalently linked to R¹ at a linkage site represented by K188 (based on Kabat numbering) and/or a linkage site represented by K190 (based on Kabat numbering) of the light chain constant region of each antibody; and
wherein in each complex, n1 is independently an integer of one or greater representing the number of instances of R¹, and optionally wherein the average value of n1 of the complexes of the composition is in the range of 1 to 5.

10. The composition of any one of claims 7-9, wherein 85%-95% of the light chain constant regions of the anti-TfR1 antibodies of the complexes in the composition are independently covalently linked to R¹ at a linkage site represented by K188 (based on Kabat numbering) and/or a linkage site represented by K190 (based on Kabat numbering) of the light chain constant region of each antibody.

11. The composition of any one of claims 7-10, wherein at least 15% of the heavy chain constant regions of the anti-TfR1 antibodies of the complexes in the composition are independently covalently linked to R¹ at a linkage site represented by K213 (based on Kabat numbering) of the heavy chain constant region of each antibody.

12. The composition of any one of claims 1-11, wherein the antibody is a Fab fragment, a full-length IgG, a Fab′ fragment, or a F(ab′)2 fragment, optionally wherein the antibody is a Fab fragment.

13. The composition of any one of claims 1-12, wherein the VH comprises an amino acid sequence at least 85% identical to SEQ ID NO: 17; and/or wherein the VL comprises an amino acid sequence at least 85% identical to SEQ ID NO: 18, optionally wherein the VH comprises the amino acid sequence of SEQ ID NO: 17 and the VL comprises the amino acid sequence of SEQ ID NO: 18.

14. The composition of any one of claims 1-13, wherein the heavy chain comprises the amino acid sequence of SEQ ID NO: 19 and the light chain comprises the amino acid sequence of SEQ ID NO: 20.

15. A method of promoting expression or activity of a dystrophin protein in a subject, comprising administering to the subject an effective amount of the composition of any one of claims 1-14.

16. A method of treating Duchenne Muscular Dystrophy (DMD) in a subject, comprising administering to the subject an effective amount of the composition of any one of claims 1-14.

17. The method of claim 15 or claim 16, wherein the subject has a mutated dystrophin allele comprising a mutation amenable to exon 51 skipping.

18. The method of claim 17, wherein the mutated dystrophin allele comprises a frameshift mutation in exon 51.

19. The method of any one of claims 15-18, wherein the complexes promote expression or activity of dystrophin protein in the subject.

20. The method of claim 19, wherein the dystrophin protein is a truncated dystrophin protein.

21. A method of determining a drug to antibody ratio (DAR) range of a first plurality of complexes, each complex comprising an antibody covalently linked to one or more oligonucleotides via a linker, wherein each linker comprises one or more protease cleavage sites, the method comprising:
(i) removing the one or more oligonucleotides from the antibodies to which they are covalently linked of the first plurality of complexes by cleaving at least one of the one or more protease cleavage sites of the linker, wherein cleaving at least one of the one or more protease cleavage sites of the linker results in a second plurality of complexes, each complex comprising an antibody covalently linked to one or more partial linkers;
(ii) obtaining the second plurality of complexes resulting from step (i);
(iii) determining the masses of the complexes obtained in step (ii) via mass spectrometry; and
(iv) determining the DAR range of the complexes obtained in step (ii); wherein a detected mass by mass spectrometry corresponding to the mass of the antibody plus the mass of n1 partial linkers indicates a DAR of n1, wherein n1 is an integer of one or greater.

22. A method of analyzing a first plurality of complexes, each complex comprising an antibody covalently linked to one or more oligonucleotides via a linker, wherein each linker comprises one or more protease cleavage sites, the method comprising:
(i) removing the one or more oligonucleotides from the antibodies to which they are covalently linked of the first plurality of complexes by cleaving at least one of the one or more protease cleavage sites in the linker, wherein cleaving at least one of the one or more protease cleavage sites in the linker results in a second plurality of complexes, each complex comprising an antibody covalently linked to one or more partial linkers, and wherein the antibody remains intact;
(ii) obtaining the second plurality complexes resulting from step (i);
(iii) digesting the antibodies of complexes obtained in (ii) with a protease to obtain fragments of the antibodies; and
(iv) determining the mass of the fragments of the antibodies obtained in step (iii) via mass spectrometry to identify the fragments covalently linked one or more partial linkers.
